(19) United States
(12) Patent Application Publication
(10) Pub. No.: US 2023/0149603 A1
(43) Pub. Date: May 18, 2023

(54) DRUG-RELEASING POLYMER COMPOSITION AND DEVICE

(71) Applicant: Novaflux Inc., Princeton, NJ (US)

(72) Inventors: Mohamed Emam Labib, West Palm Beach, FL (US); Lucas Lawrence Franz, Mount Laurel, NJ (US); Seo Yean Sohn, Princeton, NJ (US)

(21) Appl. No.: 17/986,604

(22) Filed: Nov. 14, 2022

Related U.S. Application Data
(60) Provisional application No. 63/278,595, filed on Nov. 12, 2021.

(51) Int. Cl. A61L 31/04; A61K 31/496; A61K 31/65; A61K 9/00; A61K 47/32; A61K 47/10; A61K 47/34; A61L 29/16; A61L 31/16; A61L 29/04

(52) U.S. Cl. CPC A61L 31/041; A61K 31/496; A61K 31/65; A61K 9/0024; A61K 47/32; A61K 47/10; A61K 47/34; A61L 29/16; A61L 31/16; A61L 29/049; A61L 2300/602; A61L 2300/41; A61L 2300/404; A61L 2300/216; A61L 2300/204

(57) ABSTRACT

A drug-releasing polymer composition is disclosed. It may include a major component, which may be ethylene vinyl acetate, and may further include at least one or two release-modifying materials, and may further include at least one or two drugs. The release-modifying materials may be polyethylene glycol and polycaprolactone. The drugs may be minocycline and rifampin. There may be an interaction such that in the presence of two different release-modifying materials, drug release may be greater than with either release-modifying material alone. There may be an interaction such that in the presence of two drugs, drug release may be greater than with either drug alone, and antibacterial performance may be enhanced. Release durations as long as two months are possible. In addition, the composition can be provided on a medical device that is configured for implanting in body tissue for an extended time period.

### CROSS REFERENCE TO RELATED APPLICATION

This application claims the benefit of provisional patent Application Ser. No. 63/278,595 filed with the United States Patent and Trademark Office on Nov. 12, 2021. The entire disclosure of U.S. Application Ser. No. 63/278,595 is incorporated herein by reference.

### FIELD OF THE DISCLOSURE

The present invention relates to a drug-releasing polymer composition that releases a first drug and a second drug over a time period of at least 30 days when introduced into an aqueous environment consistent with being introduced into the body and being surrounded by tissue. The present invention also relates to a medical device designed to be implanted into body tissue, wherein the medical device includes a surface layer comprising a drug-releasing polymer composition that releases a first drug and a second drug over a time period of at least 30 days.

### BACKGROUND

Various medical devices are required to penetrate a patient's skin and reside inside the body for extended periods of time. Such devices are prone to introducing or causing infection at the penetration site, and to allowing the growth of biofilm on the device or inside the site, which is very difficult to treat with systemic antibodies. For example, in the use of external bone fixators, such infections are a recognized hazard referred to as pin track infections. Entire protocols are devoted to the prevention or treatment of such infections, and even to the re-siting of pins as is sometimes required. The introduction of infection is believed to be associated with the growth of biofilm on the fixator pins, and onto surrounding tissue. Another example is that certain catheters, such as central venous, dialysis, or indwelling catheters, must be replaced at routine intervals simply because of the potential growth of biofilm on them. Devices that are entirely implanted in the body, as opposed to passing through the skin, are also subject to similar problems. Therefore, improvements in efforts to counteract bacterial introduction and biofilm formation and growth are still needed.

### SUMMARY

A composition is provided according to the present disclosure. The composition can be provided on an implantable medical device designed to be located or implanted in body tissue for an extended period of time, such as, at least about 20 days, at least about 30 days, or at least about 40 days and provide a concentration of drug each day that is greater than the minimum inhibitory concentration of a selected bacteria, and preferably provide a concentration each day that is at least 10 times the minimum inhibitory concentration of a selected bacteria.

The composition includes: a host polymeric material making up a major portion of said composition; a first release-modifying material, said first release-modifying material being mixed together with said host polymeric material, said first release-modifying material making up a minor portion of said composition; a second release-modifying material, said second release-modifying material being different from said first release-modifying material, said second release-modifying material being mixed together with said host polymeric material, said second release-modifying material making up a minor portion of said composition; a first drug; and a second drug, wherein at least some of said first drug is present in the form of discrete first particles within said composition, and at least some of said second drug is present in the form of discrete second particles within said composition, and wherein each of said drugs has a respective cumulative release of said drug from said composition to an aqueous environment that is greater than would occur with an identical composition absent said second release-modifying material.

An alternative composition includes: a host polymeric material making up a major portion of said composition; at least one release-modifying material, said first release-modifying material being mixed, together with said host polymeric material, said first release-modifying material making up a minor portion of said composition; a first drug; and a second drug, wherein at least some of said first drug is present in the form of discrete first particles within said composition, and at least some of said second drug is present in the form of discrete second particles within said composition, wherein said first drug has a respective cumulative release of said first drug from said composition to an aqueous environment that is greater than would occur with an identical composition absent said second drug, and wherein said second drug has a respective cumulative release of said second drug from said composition to an aqueous environment that is greater than would occur with an identical composition absent said first drug.

Another alternative composition includes: a host polymeric material making up a major portion of said composition, said host polymeric material being a silicone; and a first drug, wherein at least some of said first drug is present in the form of discrete first particles within said composition.

A device for implanting in body tissue comprising: and implantable device having an exterior surface and being configured for implanting in body tissue, wherein at least a portion of the exterior surface comprises a composition according to any composition described herein. An exemplary composition includes: a host polymeric material making up a major portion of said composition; a first release-modifying material, said first release-modifying material being mixed together with said host polymeric material, said first release-modifying material making up a minor portion of said composition; a second release-modifying material, said second release-modifying material being different from said first release-modifying material, said second release-modifying material being mixed together with said host polymeric material, said second release-modifying material making up a minor portion of said composition; a first drug; and a second drug, wherein at least some of said first drug is present in the form of discrete first particles within said composition, and at least some of said second drug is present in the form of discrete second particles within said composition, wherein each of said drugs has a respective cumulative release of said drug from said composition to an aqueous environment that is greater than would occur with an identical composition absent said second release-modifying material. Exemplary implantable devices include: External fixator pin cover, molded sleeve around K-wire, fixators that are transdermal, devices designed for fixation such as mandibular fixators and elbow fixators, transcutaneous catheter, orthopedic surgical equipment, sleeves for orthopedic surgical equipment, pods or other shapes to be incorporated into prosthesis, orthopedic implants, sleeve/pouch for breast implants and other implants, urinary catheter, intra-Uterine devices (IUDs) and Ancillary equipment for IUDs, catheter lock/plug, ancillary catheter equipment such as molded fittings and connectors, implants, wound cover, dermal applications such as patches where controlled release of anti-inflammatory compounds and antibiotics would be beneficial, covers of implantable screws, rods, discs plates for traumatic fracture repair, ancillary equipment for cardioverter defibrillators, drug impregnated coronary stents, covers and guards for medical equipment, incorporation in tracheal tubes for anesthesia and breathing pathways, vascular graft prosthesis, antibacterial tubing and guards for cardiopulmonary bypass, dialysis, and ECMO (extracorporeal membrane oxygenation) machines (long procedures that could use antibacterial protection; ECMO in particular has long term use), drug releasing polymer mesh bag for pacemaker, tack cover release for sacculotomy, endolymphatic shunts and ear tubes, tympanostomy tubes, ear mold/ear plugs for swimmers to prevent swimmers ear infections, endovascular shunt adaptors, suture covers/the sutures themselves, implantable clips, burr hole cover for neurosurgery, fallopian tube prosthesis, long term intravascular catheter, incorporation in breast implants, and incorporation into prosthetics.

### BRIEF DESCRIPTION OF THE DRAWINGS

Embodiments of the invention are further illustrated but are in no way limited by the illustrations herein.

FIG. 1A is a perspective, three-dimensional view of a fixation pin having a recess.

FIG. 1B is a perspective view of a fixation pin with a sleeve of an embodiment of the invention occupying the recess.

FIG. 1C is a perspective view of the fixation pin of FIG. 1B further in combination with a slidable flange.

FIG. 1D is a perspective, sectional view of a portion of the fixation pin of FIG. 1C.

FIG. 2 is a perspective view of an apparatus showing the use of fixator pins placed in relation to skin, soft tissue, and bone according to an embodiment of the present disclosure.

FIG. 3 shows a tympanostomy tube which may be made of or may comprise a composition according to the present disclosure.

FIG. 4A shows the fixator pin before the beginning of any molding operation.

FIG. 4B shows the fixator pin with temporary bushings placed on it.

FIG. 4C shows a cross section of FIG. 4B, i.e., a cross-section of the fixator pin with bushings.

FIG. 4D shows the fixator pin with bushings, placed in one half of the mold.

FIG. 4E shows the fixator pin with bushings, in closed mold (with the closer of the two mold halves being transparent).

FIG. 4F shows the fixator pin in the closed mold, with the polymer injected into the mold.

FIG. 4G shows the overmolded fixator pin and bushings pin removed from the mold.

FIG. 4H shows the mold bushings removed from the overmolded fixator pin.

FIG. 4I shows the overmolded fixator pin, with gate and sprue having been removed.

FIG. 5A1 shows a DualCap® device with isopropyl alcohol sponge.

FIG. 5A2 shows a ClearGuard HD device, with coating of chlorhexidine acetate.

FIG. 5B shows a catheter cap of composition of an embodiment of the invention.

FIG. 5C shows devices with a stylet made of composition of an embodiment of the invention.

FIG. 6A is a graph of incremental daily releases, i.e., concentration of Rifampicin released into a bath during one-day increments in an experiment, for two different compositions of Elvax.

FIG. 6B shows a comparison in release of Rifampicin between PRO-3389 and MED-6215.

FIG. 6C shows a comparison in release of Minocycline HCl between PRO-3389 and MED-6215.

FIG. 6D shows the effects of increasing drug concentration in silicone.

FIG. 7A shows the effect of adding PCL.

FIG. 7B shows Incremental (daily) release rate of rifampicin from a host polymer that is only PCL (composition is 99% PCL, 1% Rifampicin).

FIG. 8A shows Incremental release of rifampicin into a bath of distilled water.

FIG. 8B shows incremental release of minocycline into a bath of distilled water.

FIG. 8C shows cumulative release of rifampicin corresponding to FIG. 7A.

FIG. 8D shows cumulative release of minocycline corresponding to FIG. 7B.

FIG. 9A shows a similar plot of Incremental Daily Release of Minocycline.

FIG. 9B shows a similar plot of Incremental Daily Release for Rifampin.

FIG. 9C shows cumulative release of minocycline.

FIG. 9D shows cumulative release of rifampicin.

FIG. 10A shows Cumulative Release of minocycline, as a function of the square root of time.

FIG. 10B shows Cumulative Release of rifampin, as a function of the square root of time.

FIG. 11 shows cumulative release of both minocycline and rifampin from a specific blend (Blend 9), as a function of the square root of time.

FIG. 12A shows effects of polymer additives, as shown in release of minocycline, shown as cumulative Release of minocycline as a function of the square root of time.

FIG. 12B shows effects of polymer additives, as shown in release of rifampin, shown as cumulative release of rifampin as a function of the square root of time.

FIG. 13A shows Cumulative Release of rifampin in the presence or absence of the other drug, for otherwise identical formulations, shown as cumulative release of rifampin as a function of the square root of time.

FIG. 13B Release of Minocycline in the presence or absence of rifampicin in the composition, for otherwise identical formulations.

FIG. 14A shows the effect of increasing API Rifampin (Cumulative)

FIG. 14B shows the effect of increasing API Minocycline (Cumulative)

FIG. 14C shows the effect of increasing API Rifampin (Incremental)

FIG. 14D shows the data of FIG. 14C, zoomed in to Days 25-40.

FIG. 15 shows the effect of grinding rifampicin, shown as Cumulative Release of rifampin.

FIG. 16 illustrates calculation of Pin Volume.

FIG. 17A shows, in tabular format, various drug release data relative to the Minimum Inhibitory Concentration for various bacteria.

FIGS. 17B-17E show incremental release of minocycline, with secondary axes that scale the release according to the Minimum Inhibitory Concentration of four different bacteria.

FIGS. 17F-17I show incremental release of minocycline, with secondary axes that scale the release according to the Minimum Inhibitory Concentration of four different bacteria.

FIG. 17J illustrates the orientation of samples in Petri dishes.

FIG. 17K illustrates Petri dish results for Blend 9 with P. aeruginosa.

FIG. 17L illustrates Petri dish results for Blend 9 with E. coli.

FIG. 17M illustrates Petri dish results for Blend 10 with E. coli.

FIG. 17N illustrates Petri dish results for four different blends with three different bacteria.

FIG. 17O illustrates Petri dish results for three different blends with three different bacteria.

FIG. 17P illustrates Petri dish results for four different blends with three different bacteria, with variation in grinding of rifampin.

FIG. 17Q illustrates Petri dish results for three different blends with three different bacteria.

FIG. 18A shows particles of minocycline (prior to being used to make a composition of the invention).

FIGS. 18B and 18C show rifampin crystals spread on a glass slide.

FIG. 19A and FIG. 19B show polymer blends before releasing.

FIG. 19C and FIG. 19D show Polymer blends after releasing for 30 days, demonstrating pore formation during release.

FIG. 20A shows Minocycline Hydrate stored in distilled water, and Minocycline Free Base stored in distilled water.

FIG. 20B shows Minocycline Hydrate exposed to air, and Minocycline Free Base exposed to air.

FIG. 21A shows the loss of mass of Rifamycin (rifampin) at elevated temperature.

FIG. 21B shows the loss of mass of minocycline hydrochloride at elevated temperature.

FIG. 21C shows the loss of mass of minocycline free base at elevated temperature.

### DETAILED DESCRIPTION

In an embodiment, there may be provided a composition that is capable of releasing a drug when in contact with an aqueous or interstitial tissue fluid environment.

A composition according to an embodiment of the invention may comprise a host polymeric material, which may make up a major portion of the composition. It should be appreciated that a “major portion” refers to the largest component, by amount, of a composition. An example of such a material is ethylene vinyl acetate copolymer. It should be understood that other polymers including, for example, polyurethanes and silicones can be used. This polymeric material is commercially available under the name Elvax, from Dow (formerly DuPont) (Midland, Mich.). Within the Elvax family of materials, various proportions of ethylene and of vinyl acetate are available. In the examples described herein, two compositions of Ethylene Vinyl Acetate copolymer were used in different experiments. One composition used was Elvax150, which contains 32% vinyl acetate, and the balance (68%) ethylene. A second composition used was Elvax40W, which contains 40% vinyl acetate, and the balance (60%) ethylene. As the content of ethylene increases, the material becomes more hydrophobic and the melting temperature becomes higher. Elvax150 is more hydrophobic than Elvax40W and in general releases drug less rapidly than Elvax40W. Therefore, Elvax40W was used in most of the current experiments as the host polymeric material, rather than Elvax150. For other embodiments of the invention, silicone also was examined as a possible host polymer. Silicone was sourced from NuSil Technology, Avantor Sciences (Carpinteria, Calif.). Two different silicone polymers were tested, referred to as PRO-3389 and MED-6215. PRO-3389 is a 1:1 mixture ratio silicone, and MED-6215 is a 10:1 mixture. In regard to quantities of drug released, the two versions of silicone polymers did not release as much drug as Elvax40W, but can be tailored and designed to provide equivalent drug release. However, because of the extensive use and safety of silicone in various medical devices, for some embodiments of the invention, it may be desirable to use silicone polymers. In still other embodiments of the invention, the host polymeric material may be polyurethane or other biocompatible polymers.

In an embodiment, the composition may further comprise a first release-modifying material in addition to the host polymeric material, in order to modify the drug releasing properties of the host polymeric material. The first release-modifying material may make up a minor portion of the composition. It should be appreciated that a “minor portion” refers to a component that is not present as a major portion. It is believed that for at least some of the examples discussed herein, the host polymeric material and the first release-modifying material form a predominantly uniform heterogeneous material. In such a scenario, the first release-modifying material may be thought of as a pore-forming or diffusion-promoting agent which may create pathways by which drug may be conducted from the interior of the implant to its surface. The first release-modifying material may be a material that is more hydrophilic than the host polymeric material. An example of a first release-modifying material is polyethylene glycol (PEG). Other equivalent drug-releasing agents may be used including but not limited to polyvinyl alcohol and resorbable polymers without limitation.

In an embodiment, the composition may further comprise a second release-modifying material in addition to the host polymeric material and the first release-modifying material, in order to further modify the drug releasing properties of the composition. The second release-modifying material may make up a minor portion of the composition. An example of a second release-modifying material is polycaprolactone (PCL). PCL is known to be biodegradable although the time scale for its biodegradation is thought to be longer than the typical time scale of desired drug release. PEG and PCL are at least partially miscible with Elvax.

In an embodiment, the composition may further comprise a drug whose release from the composition is desired. More specifically, in an embodiment, the composition may comprise two drugs, namely, a first drug and a second drug that is different from the first drug. The two drugs may have different purposes or different target microorganisms that they are effective against. For example, one of the drugs may be effective against gram-positive bacteria while the other drug may be effective against gram-negative bacteria. As another example, one drug could be useful for inhibiting bacteria and another drug could be useful as an anti-inflammatory or for some other purpose. For example, minocycline has anti-inflammatory properties in addition to its bactericidal effects. The two drugs may have different solubilities in water or aqueous environments. The two drugs may have different degrees of hydrophilicity or hydrophobicity, or log p (octanol/water partition coefficient), as it is known in pharmaceutics or in the drug delivery field. Either drug or both of the drugs may have some degree of solubility or ability to form a mixed phase with the host polymeric material or the first release-modifying material or the second release-modifying material. Either or both of the drugs may be partially soluble in the polymer or may form a solid-solid dispersion with the host polymeric material or the first release-modifying material or the second release-modifying material.

In an embodiment of the invention, one of the drugs may be minocycline. Minocycline is a tetracycline antibiotic that is used to treat many different bacterial infections. Minocycline is effective against a wide range of bacteria, but notably does not affect Staphylococcus infections. It seems to have good effectiveness against a wide range of gram negative infections, and some gram positive infections. Minocycline is highly water-soluble. In current experiments, minocycline was present in the form of relatively small particles, with the particles being too small to discern a change of shape associated with dissolution of the drug into solid solution in the matrix (at least using optical microscopy).

Minocycline is available in both a hydrochloride form and a free base form. Either or both of these forms can be used in embodiments of the invention. The two forms have different release characteristics and different stabilities. We have found that the minocycline in the free base form seems to be less stable, seeming to oxidize and darken when in the polymer, and seeming to oxidize when dissolved in water as well. We are currently preferring the use of the hydrochloride form because it is more commonly available and seems to be more stable, even if the free base may provide a slight benefit in the form of possibly increased release rate in the early part of the release transient.

In an embodiment, the minocycline (or that portion of the minocycline that is not dissolved in other components of the composition) may be present in the form of particles.

In an embodiment of the invention, another of the drugs may be rifampin. Rifampin (also known by the names rifampicin, rifamycin, and rifadin) is a broad-spectrum antibiotic that is in a class called antimycobacterials. Rifampin is effective against gram-positive bacteria and is particularly effective against staphylococcus infections. Rifampin has low solubility in water. Rifampin has a relatively high octanol-water partition coefficient, meaning that it preferably dissolves into the oily liquid rather than into water.

In current experiments, rifampin was present in the form of relatively larger particles (larger than the particles of minocycline) that have the shape of rectangular or polygonal plates. Images of particles of Rifampin are shown herein in Example 13, illustrating a crystalline structure of Rifampin.

In an embodiment of the invention, the composition may be such that it is capable of providing release of one or both drugs, to an aqueous environment, at clinically effective concentrations, for a time duration of at least 30 days and preferably at least 40 days. It should be appreciated that a clinically effective therapeutic concentration refers to the amount needed for the drug to provide efficacy.

In embodiments of the invention, we are working with compositions that are melt processable or are able to be formed using hot melt extrusion and injection molding. This enables the production of desirable shapes that are manufacturable by those processes. Another benefit of melt processing is the higher drug loading that is achievable as a result of the presence of solid particles of drug. The common competing method is solvent impregnation, but that method is limited to lower drug contents.

In an embodiment of the invention, the various ingredients may be chosen such that the described composition is melt-processable and can be made in different shapes or sizes. For example, the host polymeric material may have a melting temperature or a temperature at which it is soft enough to be extruded, molded, formed, or otherwise processed. The first release-modifying material and the second release-modifying material do not have to be able to melt at that temperature, but they may be chosen such that they at least are able to be mixed and processed at that temperature. The drugs may be such that they do not significantly degrade or decompose at that processing temperature. For example, the drugs may be such that they have less than ˜3% degradation when exposed to 140° C. for 10 minutes, which is about the amount of time, or slightly more than the amount of time, for which the drugs are exposed to that temperature during melt mix extrusion. Either the first drug or the second drug or both may be such that they do not fully melt at that processing temperature. The lack of melting may contribute to at least some of the drug being present as discrete particles of the drug. It is believed that the presence of the drugs as discrete particles may contribute to the ability to store enough of the drug in the composition to provide long-duration release, and may contribute to the achieving of the release profiles described herein.

In an embodiment of the invention, the more-soluble drug may be more soluble in water than the less-soluble drug by a factor of at least 10 and yet the two drugs may have respective release rates from the composition to an aqueous environment that differ from each other by a factor of less than that solubility ratio.

In embodiments of the invention, it is unexpectedly found, as described elsewhere herein, that the behavior of the release rates and cumulative releases for the minocycline and for the rifampin are similar to each other. Based on this observation, in these experiments the concentration of the minocycline and the concentration of the rifampin are formulated as being equal to each other. This similarity of release rates is observed despite the fact that the minocycline is a high-water-solubility drug and was present in the form of micronized powder particles having a relatively large specific surface area, while the rifampin is a low-water-solubility drug and was present in the form of large plate-like particles having a relatively small specific surface area. This observation is both unexpected and significant, given the differing solubility, log p, and hydrophilicity characteristics of the two drugs.

It is believed possible that there is a plurality of phases in which the PEG/PCL exists in the copolymer matrix. PEG and PCL are miscible or partially miscible with Elvax. It is not wished to be limited to any particular explanation about the structure of the composition.

It should be appreciated that in a composition according to the present disclosure, the composition can contain the polymer component in an amount of about 40 wt % to 98 wt %, optionally 45 wt % to 85 wt %, optionally 50 wt % to 80 wt %, and optionally 55 wt % to 75 wt %. In addition, the composition can include the first release modifying component in an amount of 1 wt % to 20 wt %, optionally 3 wt % to 17 wt %, optionally 5 wt % to 15 wt %, and optionally 7.5 wt % to 12.5 wt %, the composition can contain the second release modifying component which is different from the first release modifying component, and which is an optional component, in an amount of 1 wt % to 20 wt %, optionally 3 wt % to 17 wt %, optionally 5 wt % to 15 wt %, and optionally 7.5 wt % to 12.5 wt %. The composition can contain the first drug in an amount of 1 wt % to 20 wt %, optionally 3 wt % to 17 wt %, optionally 5 wt % to 15 wt %, and optionally 7.5 wt % to 12.5 wt %, the composition can contain the second drug which is different from the first drug, and which is an optional component, in an amount of 1 wt % to 20 wt %, optionally 3 wt % to 17 wt %, optionally 5 wt % to 15 wt %, and optionally 7.5 wt % to 12.5 wt %. While the composition is described in the context of the polymer component, two release modifying components, and two drugs, it should be appreciated that the composition can be provided with only one release modifying agent and/or only one drug. In addition, it should be appreciated that the composition can include more that one or two different release modifying components and/or more than two different drugs.

### Medical Device Formed of the Embodiment Composition, for Use at a Penetration Through Skin

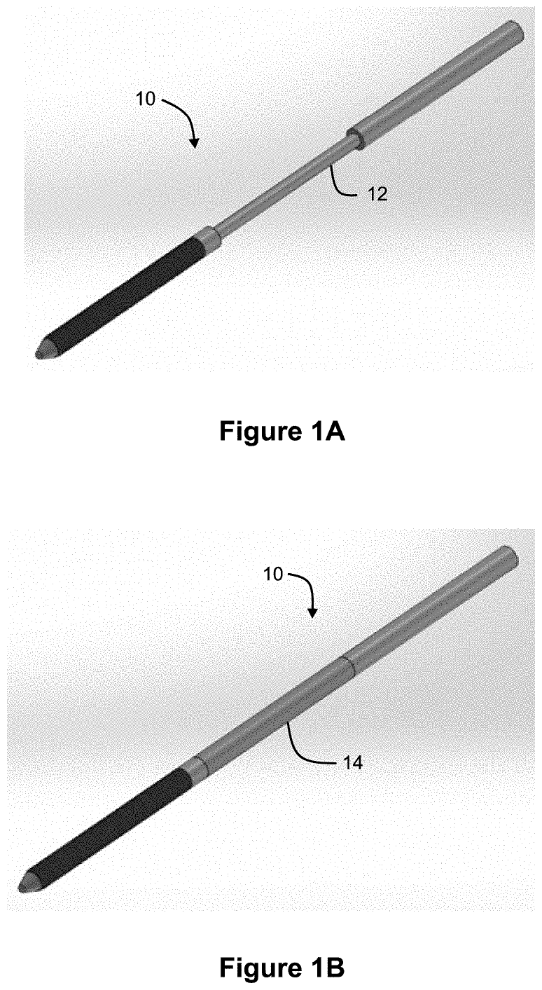

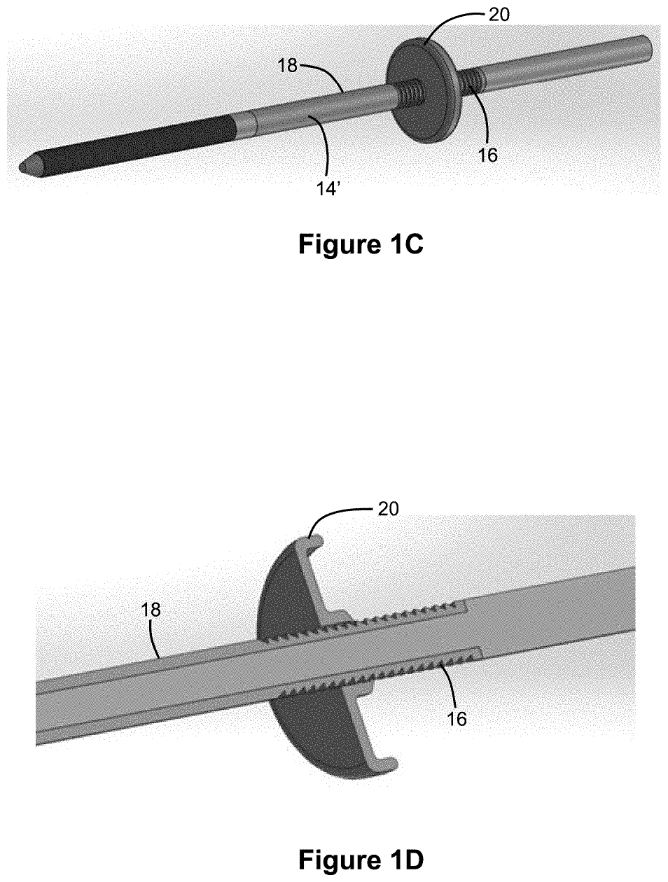

In an embodiment of the invention, the described composition may be used to form a sleeve, a coating, an enclosure, or similar device that is suitable to be placed at or near where a medical device penetrates the skin of a patient. The sleeve or similar device will be located at the interface between the device and the tissue. Examples of such skin-penetrating devices are fixator pins and wires for external bone fixators. An embodiment of the invention may have a shape that fits around or is geometrically complementary to the shape or features of the device that is placed in the patient's body or that penetrates the skin of the patient. An example of a fixator pin is illustrated in FIGS. 1A-1D.

The device shown in FIGS. 1A-1D is usable with a generally cylindrical medical device such as a fixator pin. FIG. 1A shows a fixator pin 10 having a recess 12 (of a generally annular shape) to accommodate a sleeve comprising a composition of an embodiment of the invention. FIG. 1B shows a fixator pin 10 with a sleeve 14 of an embodiment of the invention occupying the recess. The sleeve 14 may be extruded or molded in place on the fixator pin 10. In FIG. 1C, the sleeve 14′ includes external helical threads 16 on at least a portion of its external surface 18. FIG. 1C shows a slidable flange 20 that is a discrete component separate from the sleeve 14′, or it can be an integrated portion of the device. FIG. 1D shows a close-up cross-sectional view of the slidable flange 20. This is shown as comprising a Tinnerman clip configuration (sometimes referred to as a speed nut) (now available from ARaymond Tinnerman, Brunswick, Ohio). Such a device is able to engage with helical threads on the central object and is able to advance by rotation similar to a conventional nut engaging with a conventional screw. Such a device also is able to advance easily in one direction when certain tabs in the device deflect and slip past helical threads on the central object. The slidable flange may also comprise or be made of a composition of an embodiment of the invention. In FIGS. 1A-1D, the sleeves 14, 14′, including the thread 16, and the flange 20 may be formed of a composition of an embodiment of the invention.

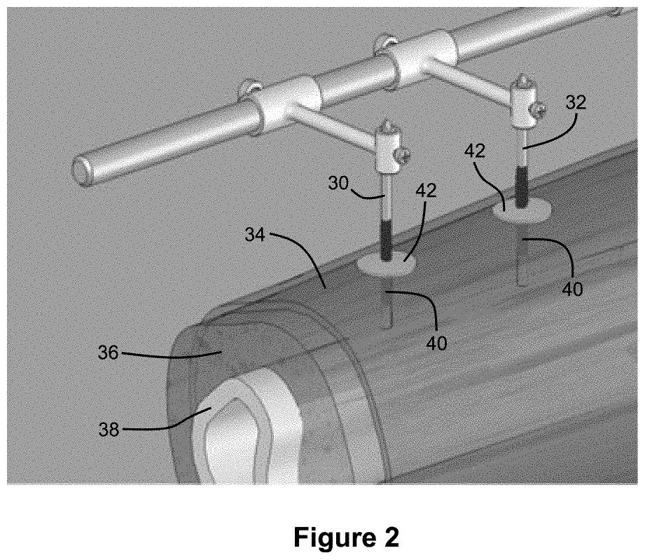

FIG. 2 shows two fixator pins 30 and 32 according to the present invention, placed in relation to skin 34, soft tissue 36, and bone 38 similar to what is done with conventional fixator pins (which typically are made of solid metal). In a portion of the pin that is intended to be near the penetration through the skin, the pin may comprise a composition 40 of an embodiment of the invention, which may be incorporated into a recess that exists in the pin. The embodiment composition may therefore continually release desired doses of drug locally along the pin track, suppressing pin track infection. This can be done while causing only slight or no systemic exposure of the patient to the drug which can be considered as regional or locoregional treatment or prophylaxis. This lack of systemic exposure is believed to minimize risk of encouraging resistance to the drug. A protective flange 42 may be attached once the pin is in place to provide further soft tissue coverage at the skin/pin interface.

In usages other than bone fixator pins, it is possible to use a similar sleeve geometry around other devices that penetrate the skin. For example, such a sleeve could be used around a catheter or other implants including prosthesis or other devices used in orthopedic or bone treatment. This could be applicable, for example, to peritoneal dialysis, hemodialysis or extracorporeal treatments, without limitation.

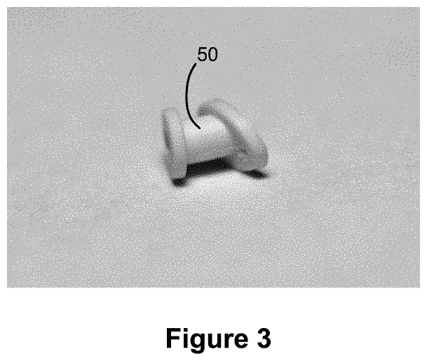

Yet another example is a tympanostomy tube such as is commonly used to treat otitis media. A tympanostomy tube 50 may be made out of or may comprise the described composition. Such a tympanostomy tube is shown in FIG. 3.

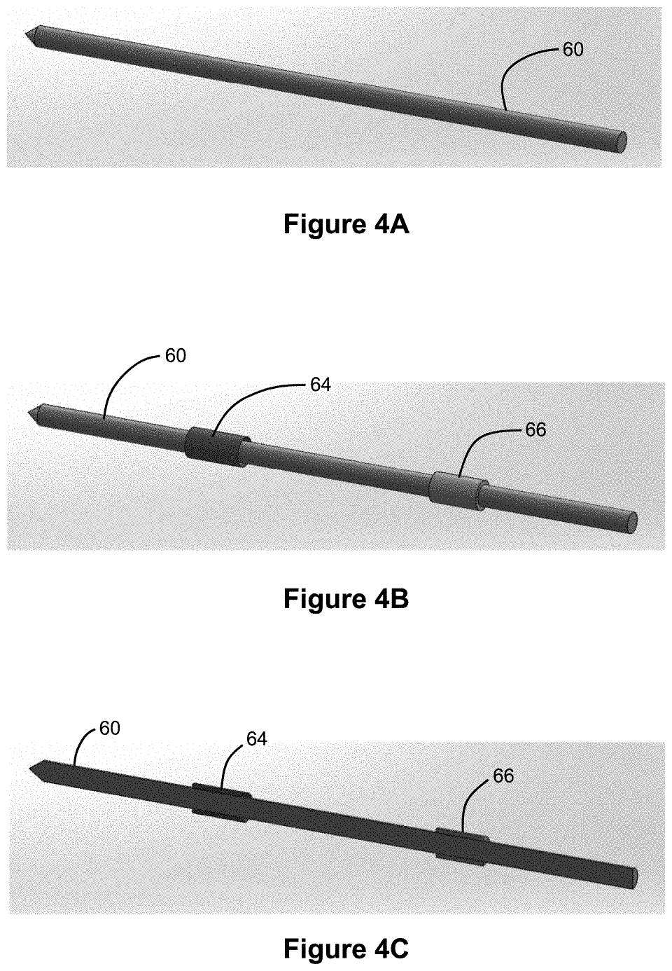

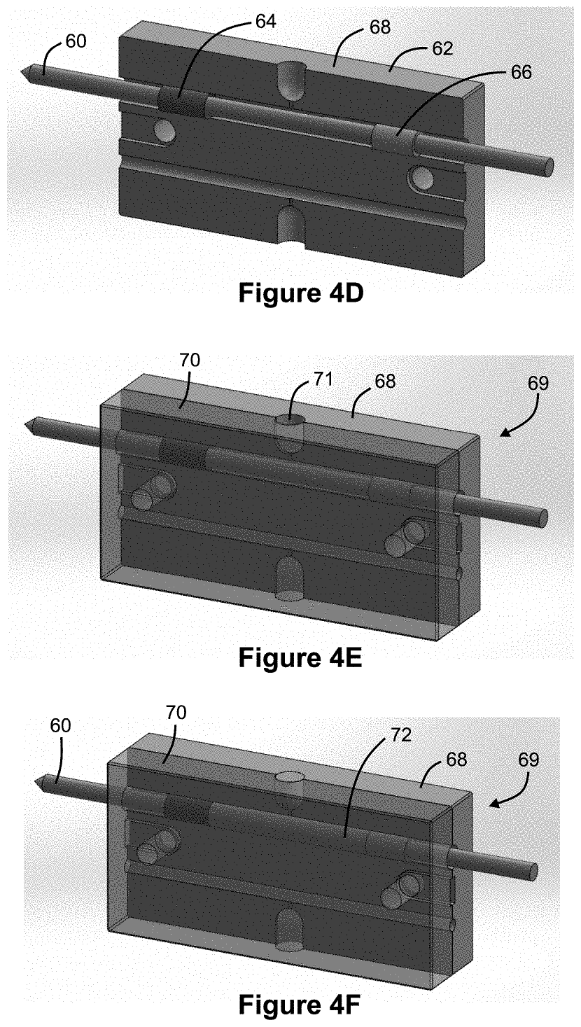

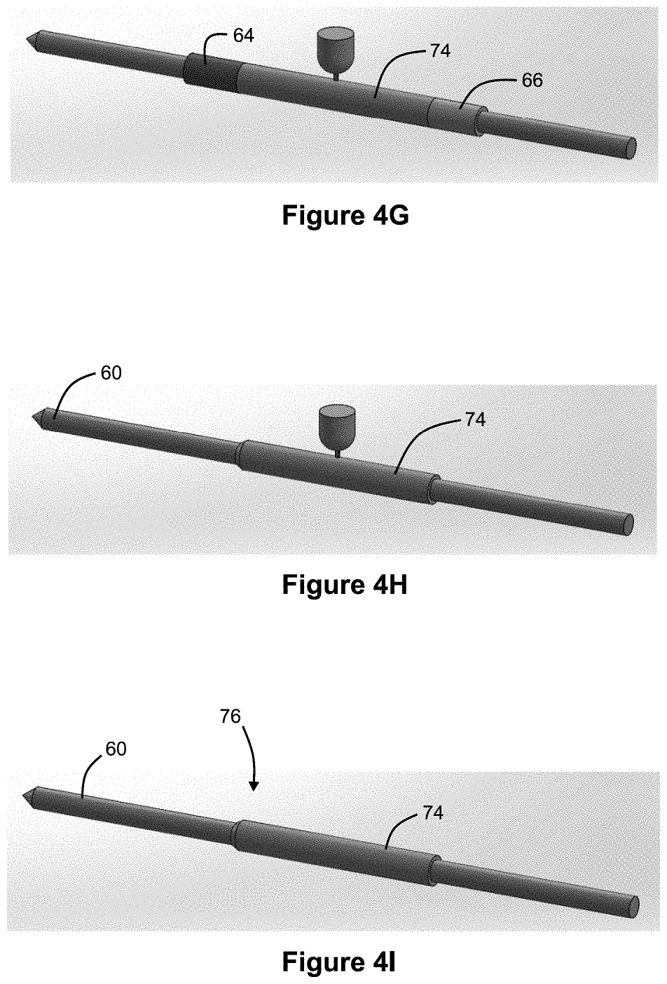

Referring now to FIGS. 4A-4I, the illustrations show a sequence of steps in a molding operation for molding a composition of an embodiment of the invention around a fixator pin 60. As illustrated in these Figures, the fixator pin 60 is a simple cylindrical pin having different thicknesses, as desired, which, as illustrated, does not have a recess therein. The mold 62 as illustrated, in its upper portion, would most likely be used to mold the composition around a Steinmann pin. A fixator pin 60 (Steinmann pin) typically has an outside diameter in the range of 5 mm. The molded material would create a sleeve or other forms surrounding the external surface of the metal pin that surrounds a portion of the pin extending out to a slightly larger diameter than the pin itself. The inventive composition may cover the entire pin or device or may be included as pods to release the intended drug.

Alternatively, it would also be possible that the pin could have a recess, and material of an embodiment of the invention could be molded into the recess.

FIG. 4A shows the fixator pin 60 before the beginning of any molding operation.

FIG. 4B shows the fixator pin with temporary bushings 64 and 66 placed on it.

FIG. 4C shows a cross section of the previous illustration, i.e., a cross-section of the fixator pin 60 with bushings 64 and 66.

FIG. 4D shows the fixator pin 60 with bushings 64 and 66, placed in one half of the mold 68.

FIG. 4E shows the fixator pin 60 with bushings 64 and 66, in a closed mold 69 of halves 68 and 70 (with the closer of the two mold halves 70 being transparent). The opening 71 is provided for introducing polymer.

FIG. 4F shows the fixator pin 60 in the closed mold, with the polymer 72 injected into the mold 69 via the opening 71.

FIG. 4G shows the overmolded fixator pin 60 and bushings 64 and 66 removed from the mold 69 with the overmold 74 thereon in a location between the bushings 64 and 66.

FIG. 4H shows the fixator pin 60 with the bushings 64 and 66 removed thereby leaving the fixator pin 60 with overmold 74 formed from the polymer 72.

FIG. 4I shows the resulting overmolded fixator pin 76, wherein the gate and sprue having been removed.

It can be noted that devices of embodiments of the invention could also be used with a K-wire (Kirschner wire). A K-wire typically has an outside diameter of about 1 mm. The mold as illustrated also contains, in its lower region, a mold cavity that is a simple small-diameter cylinder that can be used to mold the composition of the invention around a cylinder, such as a K-wire (Kirschner wire) whose diameter is smaller than the diameter of what is in the upper portion of the mold. It is likely that, in view of the already small diameter of a K-wire, the embodiment device would simply surround the exterior of the K-wire without the presence of a recess in the K-wire.

### Medical Device Formed of the Embodiment Composition, for Use as a Catheter Lock

Catheters are known sources of local and systemic infection, clotting, occlusion, patency issues, growth of biofilm, and other problems. A catheter lock solution is a liquid that is used to occupy a lumen of a catheter when the catheter does not have a flowing fluid inside it. Such catheter lock solution can be used with generally any type of catheter, including central venous catheters, urinary catheters, peritoneal dialysis catheters, hemodialysis catheters, tubes for intravenous delivery, enteral or parenteral feeding tubes, and other kinds of catheters. In some situations, it is possible that the region of interest for prevention of problems is near the end of the catheter that is suitable to connect to another component, such as in the region of a luer lock fitting. In other situations, it is possible that the region of interest extends along a substantial length of the catheter. The catheter lock solution is typically used to prevent the development of infection or clotting or biofilm growth inside or on a surface of the catheter. Examples of ingredients that may be present in catheter lock solutions include antibiotics, citrate and heparin. For such purposes, the catheter lock solution is manufactured specifically for that purpose and is supplied as a separate product and is introduced into the catheter lumen at the appropriate time by medical personnel.

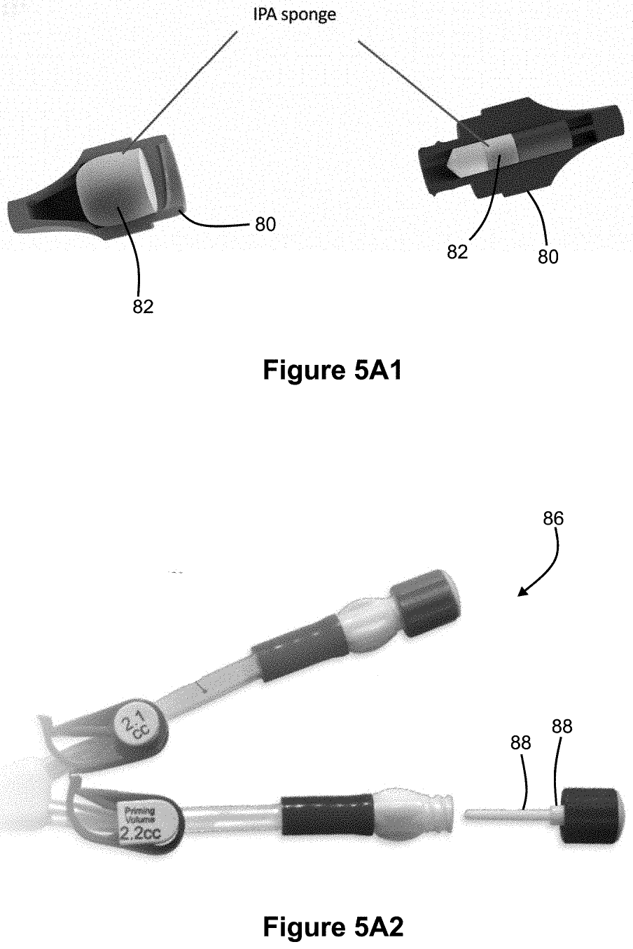

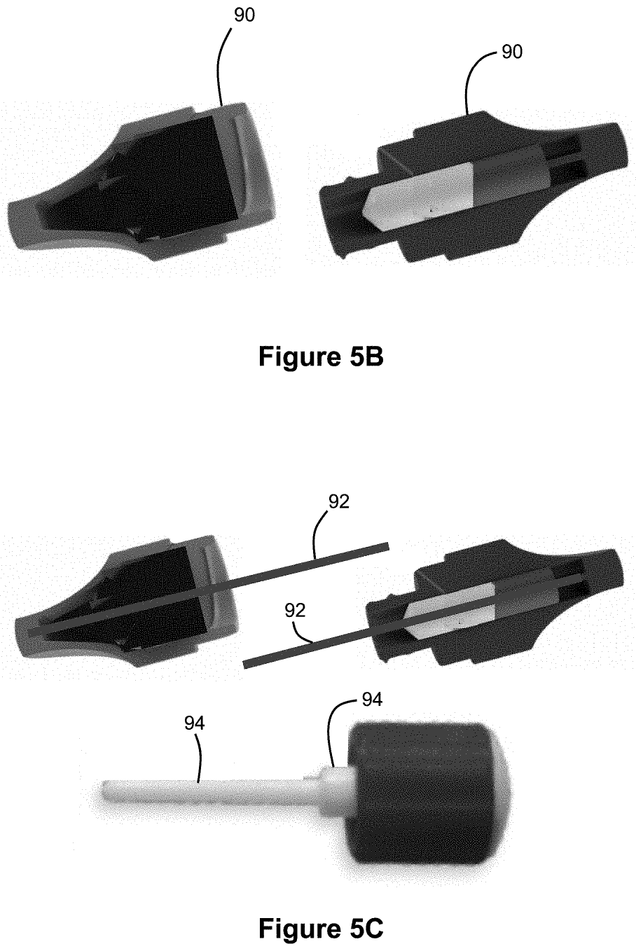

As an alternative to providing a catheter lock solution, it is possible to leave the liquid that is already inside the catheter inside the catheter, while providing a cap or similar device to close off the end of the catheter, and such cap may be effective to disinfect the end of the connector. DualCap® device, made by Merit Medical (South Jordan, Utah), is a commercially available cap for a luer connection for purposes of disinfection. The disinfecting agent is a sponge that contains 70% isopropyl alcohol. The molded plastic cap simply mechanically holds another component, the sponge, which in turn contains a disinfecting liquid within its pores. Such a device only is effective for the duration of the presence of the isopropyl alcohol. Such a device is only effective for the duration of the presence of the isopropyl alcohol and is not sufficient to prevent infections over a longer period of time, such as, for example, after the isopropyl alcohol is exhausted. FIG. 5A1 illustrates a DualCap® device 80, made by Merit Medical (South Jordan, Utah), with an isopropyl alcohol sponge 82.

Another known device is ClearGuard HD device (ICU Medical, Inc., San Clemente, Calif.), in which the antimicrobial agent is provided in the form of a coating, of chlorhexidine acetate, on the threads and also on the rod (stylet). FIG. 5A2 illustrates a ClearGuard HD device 86 with a coating of chlorhexidine acetate 88 where the rod and threads are coated with chlorhexidine, a broad spectrum antimicrobial agent.

As alternative to providing a specific separate catheter lock solution, it is possible to deliver a desired amount of a desired drug (e.g., antimicrobial or antibiotic) to the liquid inside the catheter, directly from the material of which the cap is made. In an embodiment, as illustrated in FIG. 5B, the cap 90 is made of a material described herein such as EVA, silicone, polyurethane or other material, which may include a drug or drugs in the form of solid particles or other physical form not limited to solid particles. Such delivery or release of drug can occur by dissolution, diffusion, convection, or other mechanism or combination of mechanisms. In the case of a simple cap 90, the drug-delivering material can be expected to deliver drug primarily in the immediate vicinity of the cap, and may be only in the vicinity of the clamp.

Furthermore, as illustrated in FIG. 5C, the cap could include a stylet 92 or an element that extends along the lengthwise direction of the cap and is suitable to extend into the lumen of a catheter attached to the luer connection or other connection, such as by having an outside diameter that is smaller than the lumen inside diameter.

As a result, the stylet or element can deliver drug to the region of liquid that surrounds it, which can extend some distance into the lumen of the catheter. The entire stylet 92 (or a portion of it) may comprise a drug-delivering material, which may be the same as the material of which the cap is made or may be different. In addition, the rod and the thread 94 (or a portion of it) may comprise a drug-delivering material, which may be the same as the material of which the cap is made or may be different.

In still other embodiments of the invention, a catheter, or tubing, may be made of material described herein, such as, for example, EVA, silicone, polyurethane or other polymer including blends thereof, which further may contain particles or other physical form of one or more drug, such as rifampin or minocycline or both. Such a catheter or tubing would have the ability to deliver its drug all along its length, thereby discouraging biofilm growth at all such places. Still other devices that can be made of the inventive drug-loaded silicone or polyurethane are discussed elsewhere herein. Embodiments of the invention are not limited to the polymer or blends described above.

### EXAMPLES

Embodiments of the invention are further described but are in no way limited by the Examples presented herein. Experiments were conducted using protocols and equipment as described herein. It is noted that other equipment and process can be adapted to make the compositions of the invention by persons skilled in the art.

Measurements of drug release typically are performed by producing (through extrusion or molding other methods) a sample of the composition of interest, and immersing the sample in water or an aqueous salt solution for a known immersion interval of time under mixing as it is known in the art of drug release or drug delivery. At the beginning of the immersion interval, the sink solution into which the drug is to be released (water or aqueous salt solution) does not contain any drug. At the end of the immersion interval, the concentration of the drug in the water or aqueous salt solution is measured as a function of time, which indicates how much drug release has occurred during that interval. In experiments performed herein, usually, the measurement interval was 1 day. For intervals longer than one day that were unmeasured, such as weekends, we took the total measured amount released and divided it by the measurement interval, using a spectrophotometer, as was done for all data presented here. Concentration of drug released into the sink solution is measured using a Shimadzu spectrophotometer UV-2101/3101PC. The liquid volume of bath is 10 ml, and the coupon has dimensions of 6.35 mm diameter and 1.27 mm thickness. Serial dilutions are made when required as known in the art of drug release.

Some of the graphs presented herein are graphs of incremental drug release (taken during discrete intervals that are usually one day in duration), which is essentially a drug release rate averaged over a one-day time period, or a release rate per day. These incremental drug releases are plotted as a function of time. In some of these graphs, the quantity that is plotted on the vertical axis is the concentration of the drug in the bath/sink fluid after the coupon of the embodiment composition has been immersed in the bath for a period of (typically one day). The concentration data is measured using the spectrophotometer. This quantity is proportional to the amount of drug released during the interval (typically one day), and so it can be viewed as representing a rate of release (quantity of drug released per day). In other graphs of incremental release, the quantity plotted on the vertical axis is the actual amount of drug released, in units of micrograms.

Other graphs are graphs of cumulative drug release amounts, plotted as a function of time. Information about cumulative release of a drug was obtained by mathematically summing the measured incremental releases of all previous increments for that experiment. More specifically, herein, for the plots of cumulative drug release, the horizontal axis, against which the data are plotted, is the square root of time, which is a common way of presenting such data for situations in which diffusion-release mechanism dominates transport from a polymer matrix. In general, for diffusion-dominated systems, it is theoretically expected that the cumulative release should have an approximately linear relationship with the square root of time.

It can be understood that a graph of cumulative release is essentially an integral of a graph of incremental (daily) release. Indeed, both types of graphs are obtained from the same set experimental data. The cumulative release graph is a summation adding together all of the incremental releases prior to a time point. Both types of plots are slightly stepwise, in view of the time increment (usually one day) at which measurements are taken.

For some experiments, the polymeric material and drug were processed using a single-screw extruder. For the single-screw extruder results reported here, we passed the material through that single-screw extruder four times to ensure good and uniform mixing. For most experiments, we used a Brabender extruder, which is a twin-screw extruder that is believed to mix material thoroughly with one pass of the material through the extruder. It is available from Brabender Instruments, Inc., South Hackensack, N.J. (MetaTorque Plasti-Corder, with the blades being roller blades). The only data included herein that was obtained using the single screw extruder are data from Example 4 (comparison of Elvax150 vs. Elvax40W, in which all data taken for Elvax 150 is from single screw extrusion) and Example 5 (use of two release-modifying materials in combination). All other data reported herein was obtained using the Brabender extruder.

The extrusion temperature was 140° C. and the time during which the composition was at the extrusion temperature was less than 10 minutes. Extrusion was carried out with a Brabender twin screw extruder. Compounded by weight percent, the blends are listed in the table below. The process was carried out by first weighing and mixing the polymers and drug compounds, then loading it into the Brabender and carrying out mixing until there was a uniform torque applied through the mixture. The torque first spiked when the mixture was introduced, then as the blend homogenized torque lowered. This took place at 140° C. for 8 minutes for each blend, with the exception of Compositions 10 and 14, which took 10 minutes to homogenize. The mixture was then scraped from the screws and allowed to cool on aluminum foil before storage. Material yield was 80% (˜40 g yield from 50 g raw material). The excess was lost stuck to the blades/inside of the extruder. The mixture for each blend appeared a bright red color after extrusion due to inclusion of rifampin which is red and after cooling and resting settled into a somewhat darker color. Compression molding was carried out with a carver heated lab press at 130° C. (266° F.). 10.2 grams of polymer were weighed and placed into a flat circular stainless steel die. Coupons pressed are 4″ diameter circle×1.27 mm thickness. The die and polymer are placed between two aluminum plates with a teflon sheet on each. They were then put into the lab press at around 3000 psi. Coupons were allowed to cool between the aluminum with air blasted over the surface of the metal, then, removing the aluminum, over the surface of the polymer. Coupons were pressed for each blend. Coupons appeared uniform. From these coupons, samples were cut for each release study. This pressing demonstrates ease of moldability of the polymer. This was reconfirmed by injection molding through a manual PIM-Shooter model 150 A, though all examples utilize samples used for release studies which were cut from the original pressed coupons.

### Description of Examples and Summary of Evaluated Compositions

Various experiments were performed herein using a series of experimental compositions, in which the compositions are referred to as Blends 1-20. The compositions are given in Table 1 as percent composition (by weight) of each ingredient. These compositions are used in both the Release study and the Zone of Inhibition study. Silicone compositions are not included in the table and can be found in example 1.

TABLE 1
Summary of evaluated EVA compositions

| Blend | EVA | PEG | PCL | RIF | MINO |
|---|---|---|---|---|---|
| 1 | 75% | 10% | 10% | 2.5% | 2.5% |
| 2 | 70% | 10% | 10% | 5% | 5% |
| 3 | 80% | 7.5% | 7.5% | 2.5% | 2.5% |
| 4 | 75% | 7.5% | 7.5% | 5% | 5% |
| 5 | 70% | 10% | 10% | 5% | 5%* |
| 6 | 75% | 10% | 10% | 5% | 0% |
| 7 | 75% | 10% | 10% | 0% | 5% |
| 8 | 70% | 10% | 10% | 5% | 5% |
| 9 | 60% | 10% | 10% | 10% | 10% |
| 10 | 40% | 10% | 10% | 20% | 20% |
| 11 | 80% | 10% | 0% | 5% | 5% |
| 12 | 80% | 0% | 10% | 5% | 5% |
| 13 | 90% | 0% | 0% | 5% | 5% |
| 14 | 40% | 10% | 10% | 20%** | 20% |
| 15 | 70% | 10% | 10% | 5%** | 5% |
| 16 | 84%*** | 7.5% | 7.5% | 1% | 0% |
| 17 | 84% | 7.5% | 7.5% | 1% | 0% |
| 18 | 80% | 7.5% | 7.5% | 5% | 0% |
| 19 | 80% | 15% | 0% | 5% | 0% |
| 20 | 0% | 99% | 0% | 1% | 0% |

*Free base Minocycline used
**Rifampicin has been ground
***Elvax 150 instead of Elvax 40W

From In Vitro release testing both in phosphate buffered saline and in distilled water, a summary of results can be seen tabulated below. Results shown include linearized release rate in micrograms per square root day and the total release from sample at day 30 in micrograms for each drug and each medium. (The linearized release rate is the slope of a graph of cumulative release as a function of the square root of time, which is the format of certain plots herein.)

Tables 2A-2D contain descriptive statistics used for comparing blends to each other. Linearized release rate is descriptive of the overall rate of release. It is measured by the slope of the cumulative release graph after linearization and can give a good indication of how quickly the drug content is depleted over the time of implantation. The other descriptive statistic is the total release of drug at day 30. This gives an indication of the effectiveness out to 30 days and gives some idea of the order of magnitude of drug expected when the polymer is in use. These statistics are provided for each drug in the composition and for each different set of environmental conditions.

TABLE 2A
Minocycline Summary statistics
in Phosphate Buffered Saline (PBS)

| | Linearized Release | Total Release at |
|---|---|---|
| Blend | rate MINO [ug/day^{1/2}] | day 30 MINO [ug] |
| 1 | 45.05 | 321 |
| 2 | 124.25 | 874 |
| 3 | 34.57 | 258 |
| 4 | 69.13 | 535 |
| 5 | 88.22 | 514 |
| 7 | 1.62 | 59 |
| 8 | 131.49 | 663 |
| 9 | 409.05 | 2097 |
| 10 | 399.13 | 5992 |
| 11 | 52.02 | 350 |
| 12 | 31.96 | 141 |
| 13 | 24.09 | 160 |
| 14 | 400.86 | 6972 |
| 15 | 138.13 | 694 |

TABLE 2B
Minocycline Summary statistics in
Distilled water

| | Linearized | Total |
|---|---|---|
| | Release rate | Release at |
| | MINO | day 30 |
| Blend | [ug/day^{1/2}] | MINO[ug] |
| 1 | 24.83 | 114.15 |
| 2 | 47.34 | 213.15 |
| 3 | 20.10 | 92.43 |
| 4 | 39.02 | 178.10 |
| 5 | 44.53 | 223.15 |

TABLE 2C
Rifampicin Summary statistics in
Phosphate Buffered Saline (PBS)

| | Linearized | |
|---|---|---|
| | Release rate RIF | Total Release at |
| Blend | [ug/day^{1/2}] | day 30 RIF[ug] |
| 1 | 11.74 | 141 |
| 2 | 61.07 | 506 |
| 3 | 7.79 | 104 |
| 4 | 29.48 | 302 |
| 5 | 12.16 | 153 |
| 6 | 28.53 | 143 |
| 8 | 73.40 | 378 |
| 9 | 195.93 | 1055 |
| 10 | 559.74 | 5415 |
| 11 | 51.11 | TIT |
| 12 | 40.54 | 187 |
| 13 | 27.15 | 148 |
| 14 | 475.00 | 5073 |
| 15 | 55.07 | 315 |

TABLE 2D
Minocycline Summary statistics
in Distilled water

| | Linearized | Total |
|---|---|---|
| | Release Rate | Release at |
| | RIF | day 30 |
| Blend | [ug/day^{1/2}] | RIF[ug] |
| 1 | 14.14 | 76 |
| 2 | 31.94 | 155 |
| 3 | 10.39 | 60 |
| 4 | 24.58 | 122 |
| 5 | 28.46 | 168 |
| 16 | 11.21 | 54 |
| 17 | 21.75 | 154 |
| 18 | 174.48 | 1094 |
| 19 | 26.64 | 155 |
| 20 | 10.14 | 43 |

TABLE 2E
Minocycline Summary statistics in Distilled water

| Example | | | |
|---|---|---|---|
| number | Description | Evaluated Blends | Type of study |
| 1 | Evaluation of host polymer and effects | 16, 17, all silicone | In Vitro Release Study |
| | of Vinyl acetate % in EVA as well as the | blends | |
| | usability of silicone | | |
| 2 | Evaluation of the addition of PCL | 18, 19, 20 | In Vitro Release Study |
| 3 | Release of primary compositions in | 1, 2, 3, 4, 5 | In Vitro Release Study |
| | water evaluating Drug concentration, | | |
| | Polymer additive concentration and | | |
| | Use of Free base minocycline | | |
| 4 | Release of primary compositions in | 1, 2, 3, 4, 5 | In Vitro Release Study |
| | Phosphate Buffered saline evaluating | | |
| | Drug concentration, Polymer additive | | |
| | concentration and Use of Free base | | |
| | minocycline, and environmental | | |
| | conditions | | |
| 5 | Summary of experiments 6-10, overall | 6, 7, 8, 9, 10, 11, 12, | In Vitro Release Study |
| | charts showing their release profiles | 13, 14, 15, | |
| | relative to each other | | |
| 6 | Evaluation of preferred composition | 9 | In Vitro Release Study |
| 7 | Effects of PEG and PCL on release | 8, 11, 12, 13 | In Vitro Release Study |
| 8 | Effects of the release of one drug in | 6, 7, 8 | In Vitro Release Study |
| | the presence or absence of the other | | |
| 9 | Effect of concentration on the release | 8, 9, 10 | In Vitro Release Study |
| | of Minocycline and Rifampicin | | |
| 10 | Effects of grinding rifampicin on | 8, 10, 14, 15 | In Vitro Release Study |
| | release | | |
| 11 | Comparison of blend 9 release data to | 9 | Microbiology |
| | minimum inhibitory concentration of | | |
| | S. Aureus S. Epidermidis, A. Baumannii, | | |
| | and E. Coli | | |
| 12 | Zone of inhibition study confirming | 6, 7, 8, 9, 10, 11, 12, | Microbiology |
| | results of experiments 5-10 with the | 13, 14, 15, | |
| | polymer's inhibition of bacterial | | |
| | growth | | |
| 13 | Drug Particle size and characteristics | — | Material |
| | | | Characterization |
| 14 | Evidence of solid solution formed in | — | Material |
| | polymer and of pore formation during | | Characterization |
| | release | | |
| 15 | Comparison of Free base minocycline | — | Material |
| | with Minocycline Hydrochloride | | Characterization |
| 16 | DSC and TGA studies on drug particles, | — | Material |
| | demonstrating lack of degradation at | | Characterization |
| | processing temperature | | |

### Example 1: Host Polymeric Material: Comparison Regarding Two Different Grades of Elvax and Use of Silicone Polymer Host

As described elsewhere herein, ethylene vinyl acetate copolymer is commercially available under the name Elvax, and two specific commercially available compositions are Elvax150 (32% vinyl acetate comonomer, balance ethylene) and Elvax40W (40% vinyl acetate comonomer, balance ethylene). In this Example, the release properties of two compositions of Elvax were investigated and compared by preparing each composition of Elvax also including two release-modifying materials as described elsewhere herein and also including a chosen concentration of one drug. The composition tested here was: drug content 1% Rifampicin; first release modifying material PEG at a concentration of 7.5%; second release modifying material PCL at a concentration of 7.5%; balance Elvax, which was either Elvax 40W or Elvax 150. These additives and concentrations are reported also in various other examples herein and are constituents and concentrations used for this experiment were typical of the constituents and concentrations that are of interest for present purposes.

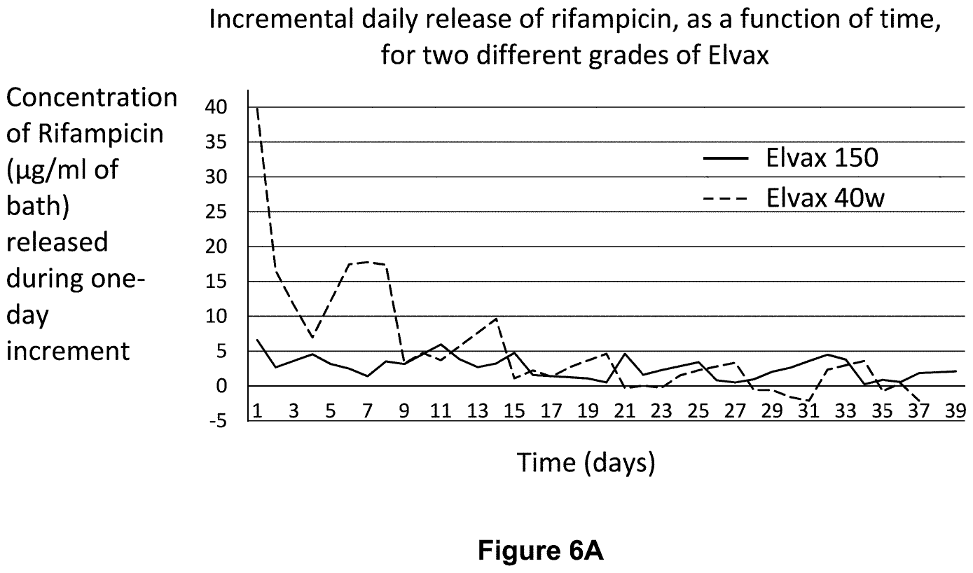

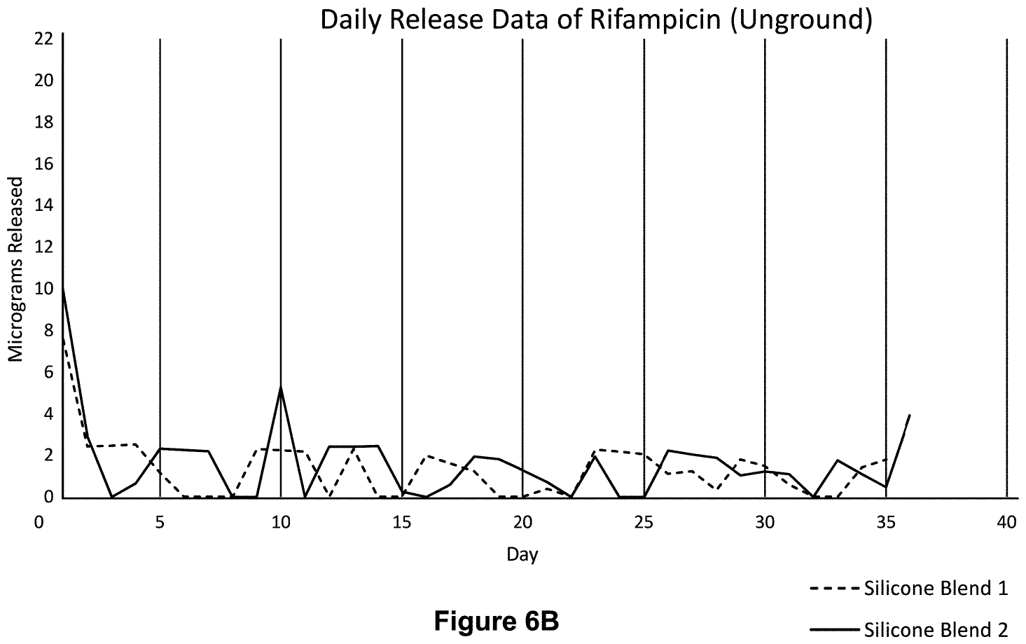

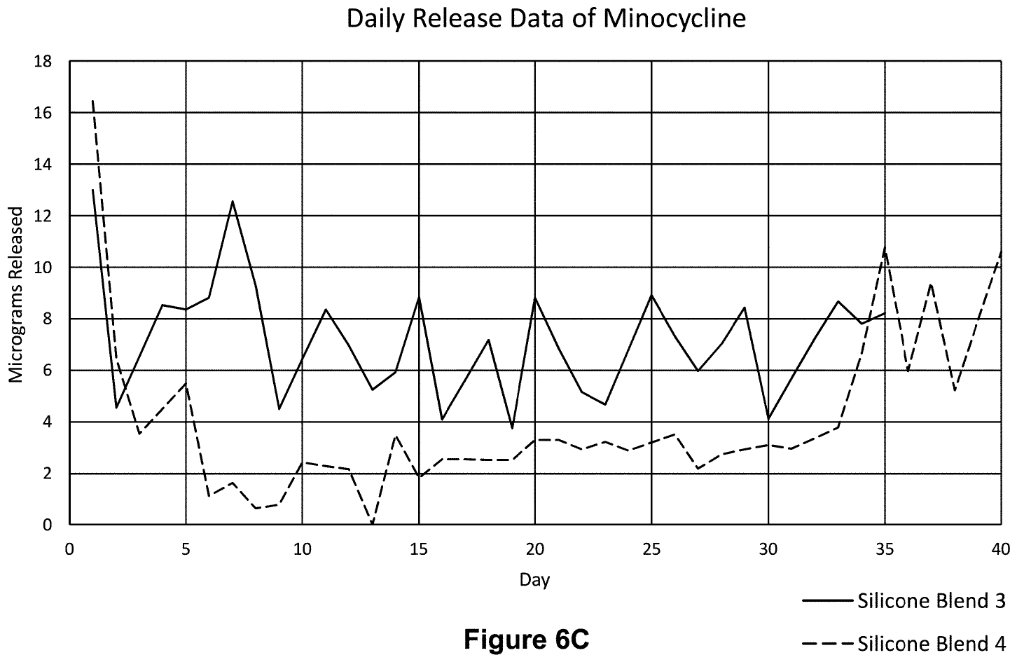

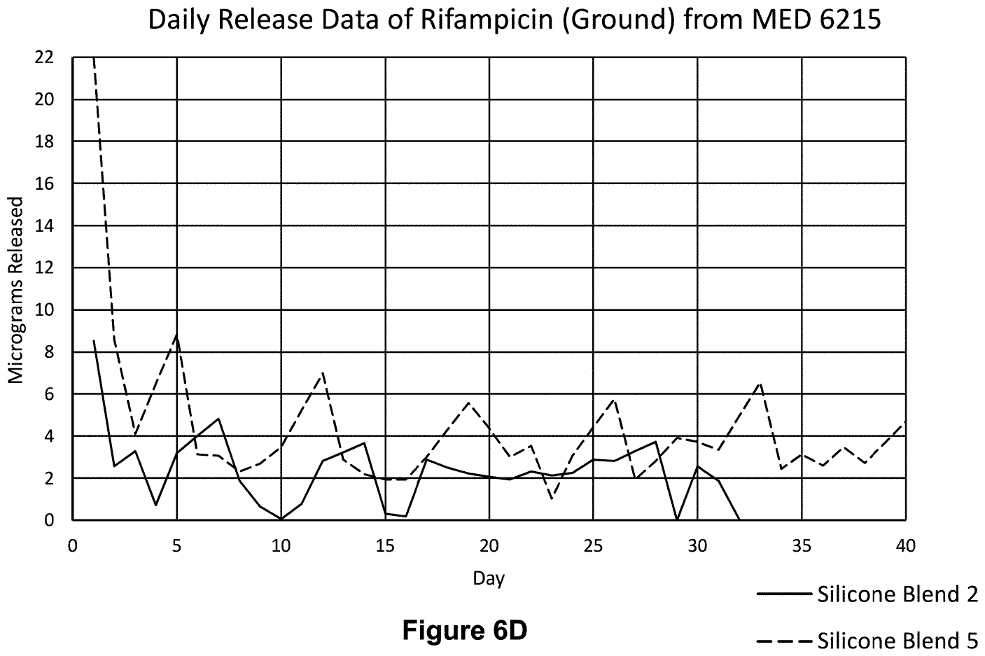

FIG. 6A shows the experimental results comparing incremental drug release (for one-day intervals of time) from the two compositions of Elvax. Comparing these results for a composition of Elvax150 to the results for a composition of Elvax40W, it appears that for the first 20 days, the incremental release from Elvax40W was significantly larger than the incremental release from Elvax150. During the time period after 20 days, the incremental releases from the two forms of Elvax were of similar magnitude to each other. Therefore, other than for this one comparative experiment, the data reported herein involving Elvax was performed using Elvax40W. A summary of the data is reported in Table 3.

TABLE 3

| | | | | | | Linearized | Total Release |
|---|---|---|---|---|---|---|---|
| | | | | | | Release Rate | at day |
| Blend | EVA | PEG | PCL | RIF | MINO | RIF [ug/day^{1/2}] | 30 RIF[ug] |
| 16 | 84%*** | 7.5% | 7.5% | 1% | 0% | 11.21 | 54 |
| 17 | 84% | 7.5% | 7.5% | 1% | 0% | 21.75 | 154 |

***Elvax 150, instead of 40W

In some experiments, silicone was also used as a host polymer incorporating minocycline and rifampicin. Two different silicone hosts were tested and are referred to as PRO-3389 and MED-6215. Both are two part silicones. PRO-3389 is a 1:1 mixture ratio and MED-6215 is a 10:1 mixture ratio. PRO-3389 is a 1:1 mixture ratio silicone, and MED-6215 is a 10:1 mixture. The mixture ratio refers to part A (as designated by the manufacturer) to part B (which is described by the manufacturer as siloxanes and silicones, dimethyl, methyl hydrogen, and octamethylcyclotetrasiloxane). Samples were prepared by mixing the first part of the silicone elastomer with the drug compound in a petri dish and then stirring in the second part, before leaving to cure. Samples with 1% drug were cured at 80° C. on a hot plate for 15-25 minutes. 5% drug composition was cured at 68° C. for 20 minutes. Silicone samples exhibited notable release and are usable as a host polymer, especially in the 5% polymer. Release graphs for the blends in the table below are carried out in distilled water.

TABLE 4
silicone compositions and summary statistics

| | | | | | Linearized | Total Release |
|---|---|---|---|---|---|---|
| Silicone Blend | | Silicone | | | Release Rate | at day |
| Number | Silicone | type | RIF | MINO | [ug/day^{1/2}] | 30 [ug] |
| 1 | 99% | PRO-3389 | 1% | 0% | 7.97 | 42.69 |
| 2 | 99% | MED-6215 | 1% | 0% | 9.76 | 49.56 |
| 3 | 99% | PRO-3389 | 0% | 1% | 50.4 | 212.71 |
| 4 | 99% | MED-6215 | 0% | 1% | 24.01 | 96.12 |
| 5 | 95% | MED-6215 | 5% | 0% | 28.90 | 136.28 |

It can be seen from the FIGS. 6B and 6C that release rate of minocycline was greater than that of rifampicin from silicone as well as from the summary statistics provided in the composition table. Total release of rifampicin was observed to be lower than that of EVA with polymer additives, release promotors in silicone could improve this characteristic. It was observed that PRO-3389 was significantly better at releasing minocycline and MED-6215 was better at releasing Rifampicin. Rifampicin is not water soluble and is the more difficult of the two to release from the host, so MED-6215 may be more suitable for some applications, while if more minocycline is necessary PRO-3389 should be more heavily considered. Further studies focus on EVA 40W as the primary host polymer since at 1% rifampicin concentration it performed the best in terms of release of evaluated samples. Each host polymer may be suitable for different applications, as all tested polymers showed some release in this example.

### Example 2: Use of Two Release-Modifying Materials in Combination

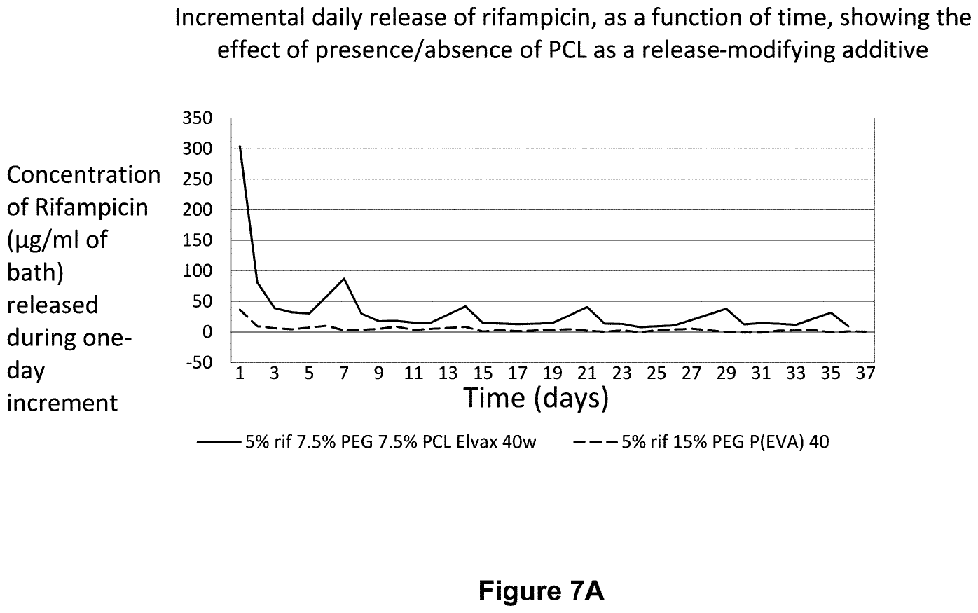

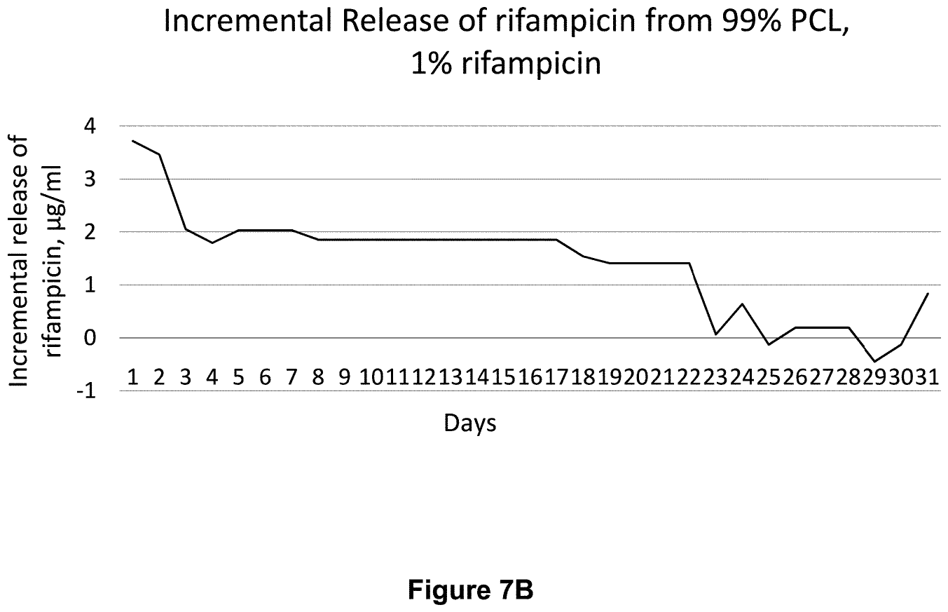

It is known in the art that, in combination with a polymeric host material that is Elvax, polyethylene glycol (PEG) functions as a release-modifying material. The PEG increases the drug release rate, compared to what the release rate would be for Elvax alone. In the current work, we wanted even a larger release than was available from Elvax+PEG. So, we tried the combination with an additional additive, Elvax+PEG+polycaprolactone (PCL). Adding PCL, i.e., using a composition comprising Elvax+PEG+PCL, seems to increase release rate compared to Elvax+PEG alone. In the comparison reported in this Example, the total concentration of release-modifying polymer is the same for both tested compositions. In one composition there was a 15% concentration of PEG. In the other composition, there was a 7.5% concentration of PEG and a 7.5% concentration of PCL. This is reported in FIG. 7A.

In the work reported here, when our compositions contain both PEG and PCL ingredients, we use equal proportions of each. We consider it to be a surprising and fruitful result that having PCL+PEG causes a substantial increase in drug release, compared to the release rate from the presence of PEG alone in a concentration that is equal to the total of PEG+PCL in comparable case.

It appears that the total concentration of release-modifying polymer is not determinative of release performance, but rather the combination of two release-modifying polymers produces larger release than all-PEG in a concentration equal to the total concentration of the two release-modifying polymers.

It can be speculated that the PCL biodegrades or biosors and forms release channels resulting from its biodegradation. However, the time frame of these experiments is 20 to 40 days, and the general knowledge about PCL is that although PCL is biodegradable, the rate at which it degrades is too slow for it to change or biodegrade very much during a 20 or 40 day period of experimentation or use. This suggests that the PCL is not functioning by hydrolysis forming channels, but rather is functioning by some other unidentified mechanism.

Inclusion of PCL seems to increase drug release, but it is not wished to be limited to attributing the increase to any particular physical mechanism.

As a separate but related investigation specifically about PCL, an experiment was conducted using pure PCL as a polymer host (with no Elvax present) but with one drug present. It was found that using pure PCL as a polymer host does not result in good release characteristics. Incremental release of drug (rifampicin) from pure PCL as a polymer host is shown in FIG. 7B. The composition was 99% PCL, 1% Rifampicin. The incremental daily release never exceeds 4 micrograms per day, and release only occurs for about 22 days. This suggests the PCL is not itself an inherently high-release material, but rather that there is something about the composition of PCL in combination with Elvax and PEG, such that apparently PCL has some effect on the polymer matrix of EVA and PEG to further influence drug release.

The data reported in FIGS. 7A and 7B can be found in Table 5.

TABLE 5

| | | | | | | Linearized | Total |
|---|---|---|---|---|---|---|---|
| | | | | | | Release | Release |
| | | | | | | Rate | at day |
| | | | | | | RIF | 30 |
| Blend | EVA | PEG | PCL | RIF | MINO | [ug/day^{1/2}] | RIF[ug] |
| 18 | 80% | 7.5% | 7.5% | 5% | 0% | 174.48 | 1094 |
| 19 | 80% | 15% | 0% | 5% | 0% | 26.64 | 155 |
| 20 | 0% | 99% | 0% | 1% | 0% | 10.14 | 43 |

### Example 3: Experiments Using Blend 1-Blend 5 in Distilled Water

First, release experiments were conducted using Blend 1 through Blend 5 in distilled water. These experiments were conducted to identify general trends and select compositional ranges of certain ingredients, as a starting point for still later experiments.

Each of the two drugs used in present experiments (Rifampin and Minocycline) addresses a different category or class of bacteria according to Gram classification, either Gram positive or Gram negative. When the two drugs are delivered together, the combination provides a broad spectrum of treatment against a variety of bacteria which is significant to the purpose of the devices described herein.

Minocycline is water-soluble, while rifampin has quite low solubility in water. The difference between the two drugs in water solubility is slightly more than one order of magnitude. This by itself might suggest some difference in release between the two drugs. However, in our experiments so far, the particle sizes of the two drugs were inconsistent with each other, so that making a direct comparison is perhaps not available yet. In fact, the direction in which the particle sizes of the two drugs differ from each other makes it even more surprising that the release rates of the two drugs were observed to be as similar as they were.

In our experiments so far, the minocycline (which is a high-water-solubility drug) was present in the form of micronized powder particles having particle sizes around 10 microns, which means that the minocycline has a relatively large specific surface area. Both of these factors seem like they should promote a relatively large release rate for minocycline. On the other hand, the rifampin (which is a low-water-solubility drug) was present in the form of larger plate-like particles having particle size around 100 microns and therefore having a relatively smaller specific surface area. Both of these factors seem to suggest an expectation of a relatively small release rate for rifampin.

Nevertheless, the releases of minocycline and rifampin (present together in the same samples) were measured to be fairly similar to each other. It is not understand why, and it appears to be unexpected, possibly indicating a synergy between the release of the two drugs. Of course, this explanation or theory is not binding herein.

The data presented in this Example were mixed in the Brabender mixer/extruder. These results are not normalized by coupon mass or surface area. These results are for samples that contain:
Host polymer=Elvax40W
Release-modifying polymer is present in equal concentrations of PEG and PCL (if both are present), i.e., either 10% of each or 7.5% of each
Drug is present in roughly equal concentrations of Rifampin and Minocycline (if both are present), i.e., either 2.5% or 5% of each drug
In various different formulations, Minocycline is present either in HCl form or in Free base form

One finding from our various experiments reported herein is that the release rates of minocycline and rifampin have the same general shape or release profile as each other, with respect to time. The cumulative release of minocycline is about double the cumulative release of rifampin. It is possible that there is some synergy between the two drugs (Rifampin and Minocycline) in regard to the release characteristics. It is not known what the mechanism is or whether the synergy can be generalized to other drug combinations.

In present circumstances, compared to rifampicin, the minocycline is intrinsically more water-soluble by slightly more than an order of magnitude and it also is present in smaller particles, so intuitively it might be expected that the release of minocycline would be much faster than the release of rifampicin. However, experimentally it is found that the minocycline release is only slightly faster than the rifampicin release and in fact is almost comparable. So, when there is comparable release between a drug with a very small particle size and a higher solubility in water, it is a significant and unexpected result since we would expect a greatly higher release of the minocycline from our polymer matrix.

Following are incremental daily releases for Rifampicin and minocycline from a sample that contains both minocycline and rifampicin.

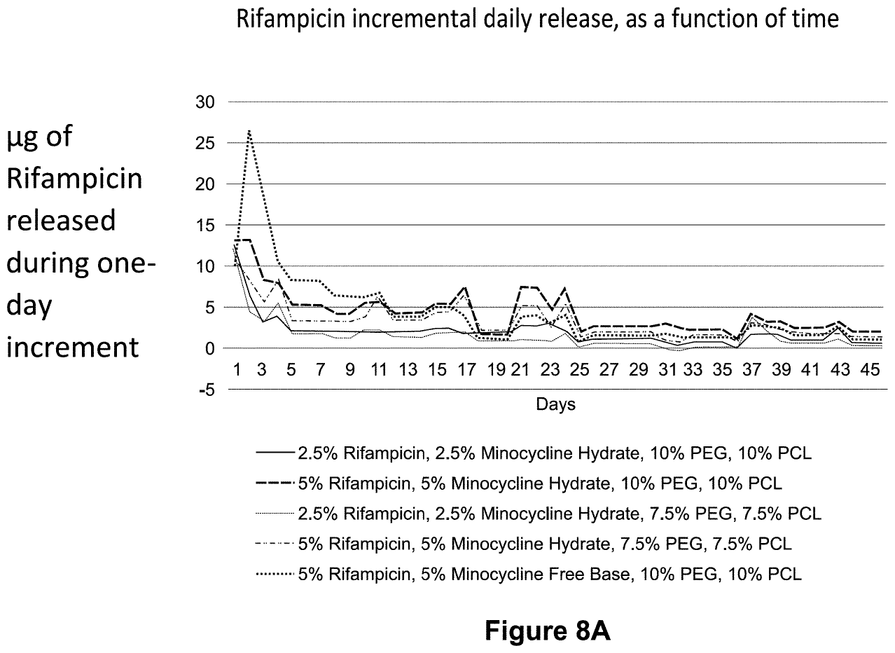

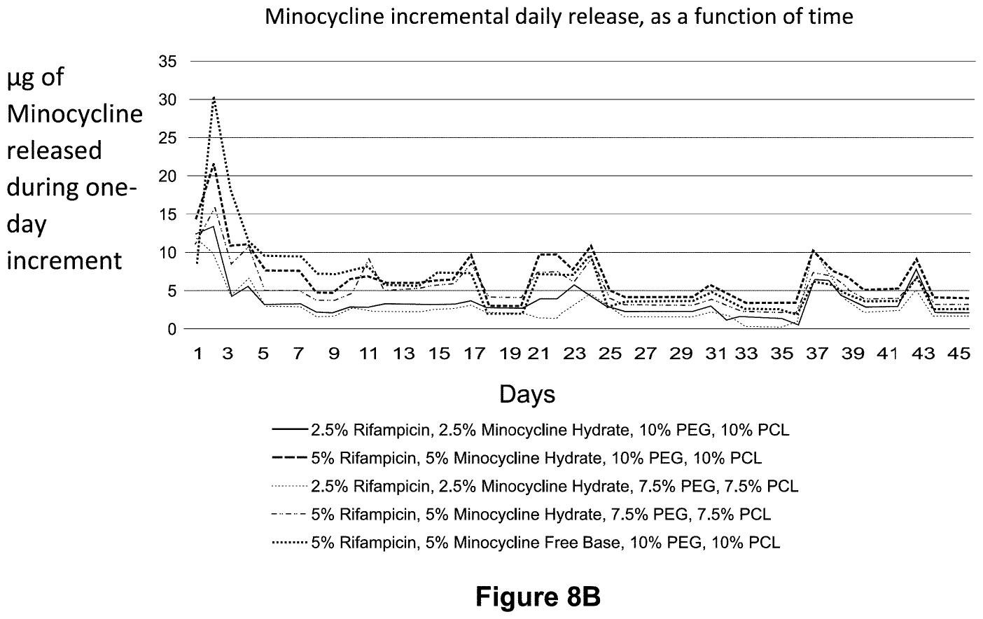

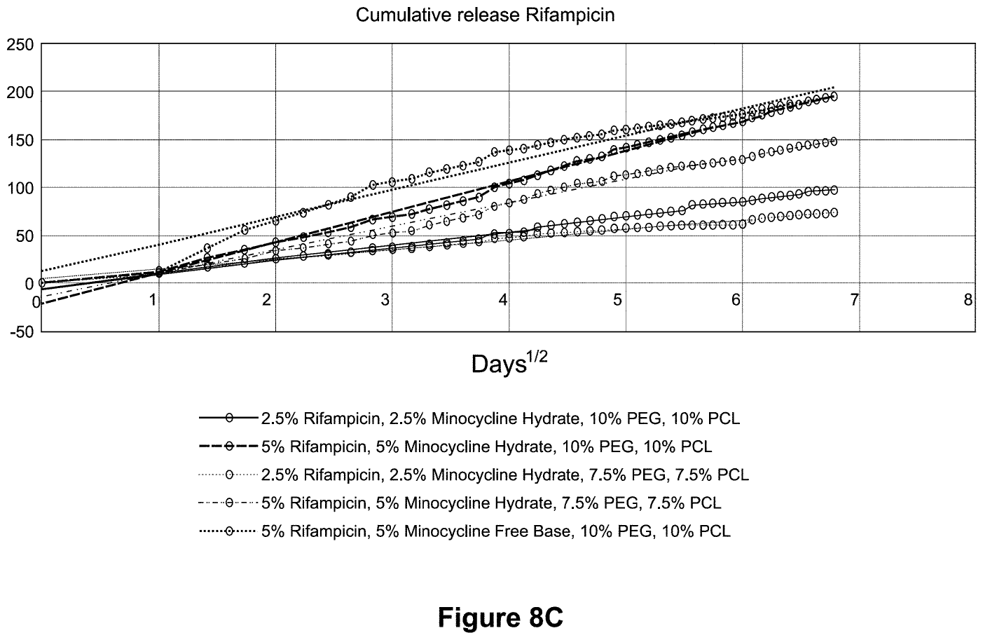

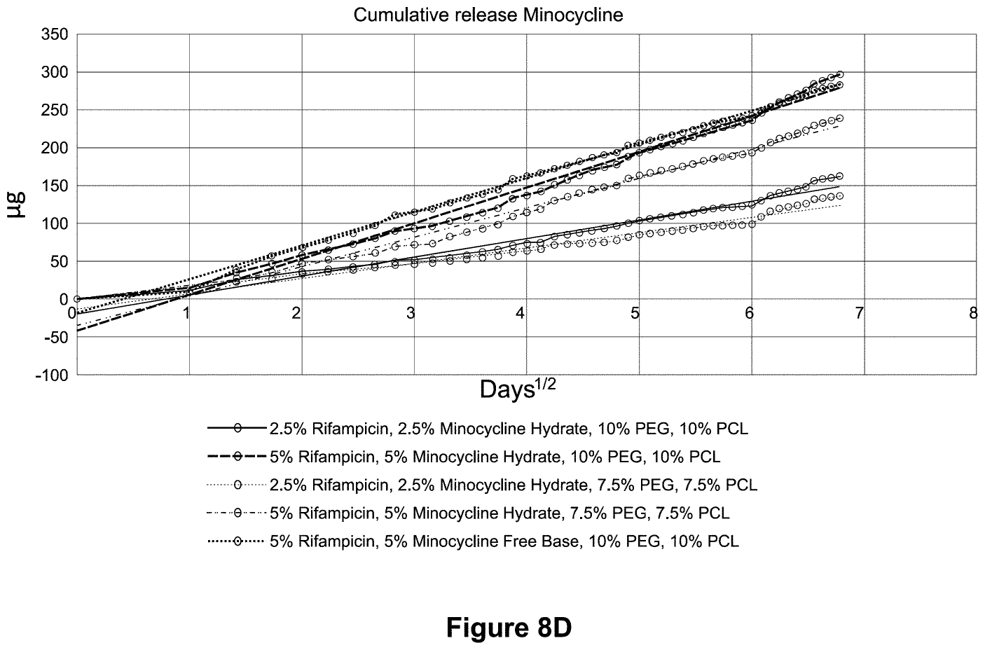

FIG. 8A shows incremental Rifampin release from a composition that contains both drugs.

FIG. 8B shows incremental Minocycline release from a composition that contains both drugs.

First, presented is incremental daily release, which is essentially a release rate with a resolution of one day, plotted as a function of time.

FIG. 8C shows cumulative release of rifampicin plotted against square root of time.

FIG. 8D shows cumulative release of minocycline plotted against square root of time.
For this set of compositions (compositions 1-5), the blend with the most desirable release characteristics for both minocycline and rifampicin is found to be 5% concentration of Rifampicin, 5% concentration of Minocycline HCl and 10% concentration of both PEG and 10% concentration of PCL. This blend has the largest release over time. Specifically, in regard to a comparison between minocycline free base and minocycline hydrochloride, the minocycline free base released more in the beginning of the study, and over longer periods of time the minocycline hydrochloride had larger release.
In general, as might be expected, larger concentrations of PEG and PCL leads to larger release of drug. For example, in all formulations, 7.5% PEG and PCL released less drug than 10% of PEG and PCL.
Release is Diffusion dominated as demonstrated by the cumulative release being approximately linear when plotted against the square root of time.
The data is reported in Table 6.

TABLE 6

| | | | | | | Linearized | Total | Linearized | Total |
|---|---|---|---|---|---|---|---|---|---|
| | | | | | | Release | Release at | Release Rate | Release at |
| | | | | | | rate MINO | day 30 | RIF | day 30 |
| Blend | EVA | PEG | PCL | RIF | MINO | [ug/day^{1/2}] | MINO[ug] | [ug/day^{1/2}] | RIF[ug] |
| 1 | 75% | 10% | 10% | 2.5% | 2.5% | 24.83 | 114.15 | 14.14 | 76 |
| 2 | 70% | 10% | 10% | 5% | 5% | 47.34 | 213.15 | 31.94 | 155 |
| 3 | 80% | 7.5% | 7.5% | 2.5% | 2.5% | 20.10 | 92.43 | 10.39 | 60 |
| 4 | 75% | 7.5% | 7.5% | 5% | 5% | 39.02 | 178.10 | 24.58 | 122 |
| 5 | 70% | 10% | 10% | 5% | 5%* | 44.53 | 223.15 | 28.46 | 168 |

Referring now to FIG. 8A, there is shown Incremental (Daily) Release of Rifampicin into distilled water at 20° C. (unstirred). Release data includes data from five blends (Blend 1-Blend 5) having varying concentration of API and concentration of PEG/PCL. This demonstrates the effects of polymer additives (release from 10% PEG/PCL>release from 7.5% PEG/PCL). This also demonstrates the effects of increasing Rifampicin content from 2.5% to 5%. (Release from a composition containing 5% Rifampicin is greater than release from a composition containing 2.5% rifampicin.)

Referring now to FIG. 8B, there is shown a similar plot of Incremental (Daily) Release of Minocycline into distilled water at 20° C. Release data includes that from five blends varying concentrations of API (Active Pharmaceutical Ingredient) and concentrations PEG/PCL. This demonstrates the effects of polymer additives (10% PEG/PCL>7.5% PEG/PCL.) This also demonstrates the effects of increasing API (Active Pharmaceutical Ingredient) content from 2.5% to 5% (Release from a composition containing 5% minocycline is greater than release from a composition containing 2.5% minocycline.)

Referring now to FIG. 8C and FIG. 8D, there are presented the same data as FIGS. 8A and 8B in the form of cumulative releases. The releases are plotted as a function of time, more specifically as a function of square root of time. This form of plotting is appropriate for use in regard to processes that are diffusion-dominated processes, because of a theoretically expected linear relationship between cumulative release and square root of time.

FIGS. 8C, 8D: Cumulative release of rifampicin and minocycline respectively. This demonstrates diffusion dominated release. This demonstrates the larger initial release of free base minocycline and a Minocycline HCL release that is more steady or consistent. The line for 5% Rifampicin, 5% Minocycline Hydrate, 10% PEG, 10% PCL overtakes the line for 5% Rifampicin, 5% Minocycline F over time. This could be due to instabilities in the free base minocycline as demonstrated in Example 15.

Based on the data reported in Table 6, we concluded that, in regard to the release-modifying additives, a preferred combination and concentration is 10% concentration of PEG and 10% concentration of PCL (as represented by Blend 1, Blend 2, Blend 5) is preferable, rather than a 7.5% concentration of PEG and a 7.5% concentration of PCL (as represented by Blend 3, Blend 4).

From this, we concluded that, in regard to concentration of API (with the concentrations of minocycline and of rifampin being equal to each other), a drug concentration of 5% for each drug is preferable to a 2.5% concentration of each drug, because it gives a larger release which is desirable. Actually, based on these results, Blend 6-Blend 15 were formulated to contain still larger concentrations of drug, in some cases.

From this, we also made a comparison about minocycline free base vs minocycline hydrochloride. Blend 5 and Blend 2 were the same (each having a 5% concentration of minocycline), except that in Blend 2 the minocycline is the hydrochloride form, while in Blend 5 the minocycline is the free base form. In these cumulative release results, it can be seen that in the early part of the release experiment (up to day 36 approximately), the freebase form (Blend 5) gives larger release of minocycline, while in later days the cumulative release of Blend 5 drops below that of Blend 2. We are not sure if this is due to degradation (we should not have already dumped all of our drug to the point of reaching depletion). So, it is possible that if a priority is large release early in the transient, then freebase would be good; nevertheless, for present purposes we are more concerned about longer-term release, so in later experiments we designate Blend 9 (which contains minocycline hydrochloride) as a preferred composition which is used extensively in later experiments. The minocycline hydrochloride also has an advantage in terms of stability.

### Example 4: Experiments Using Blend 1-Blend 5 in Phosphate Buffered Saline at 37° C.

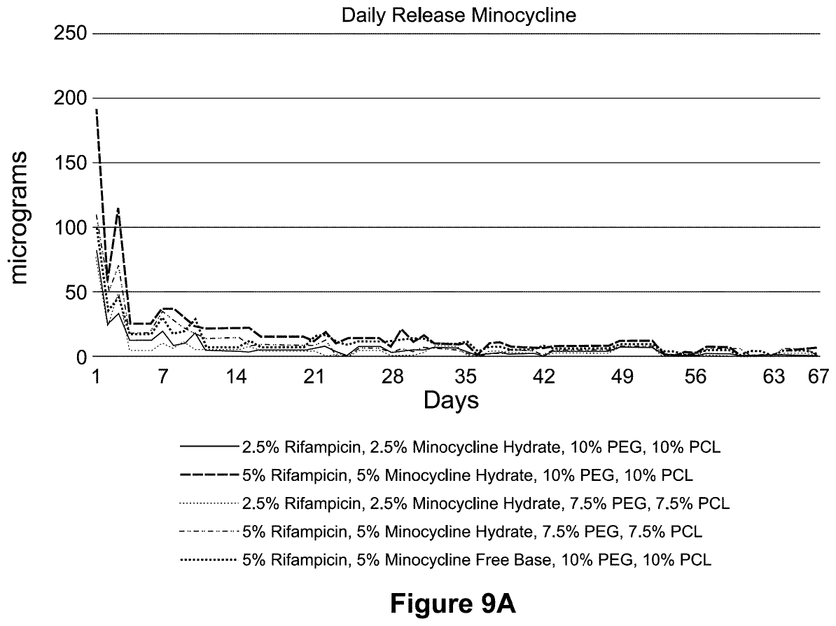

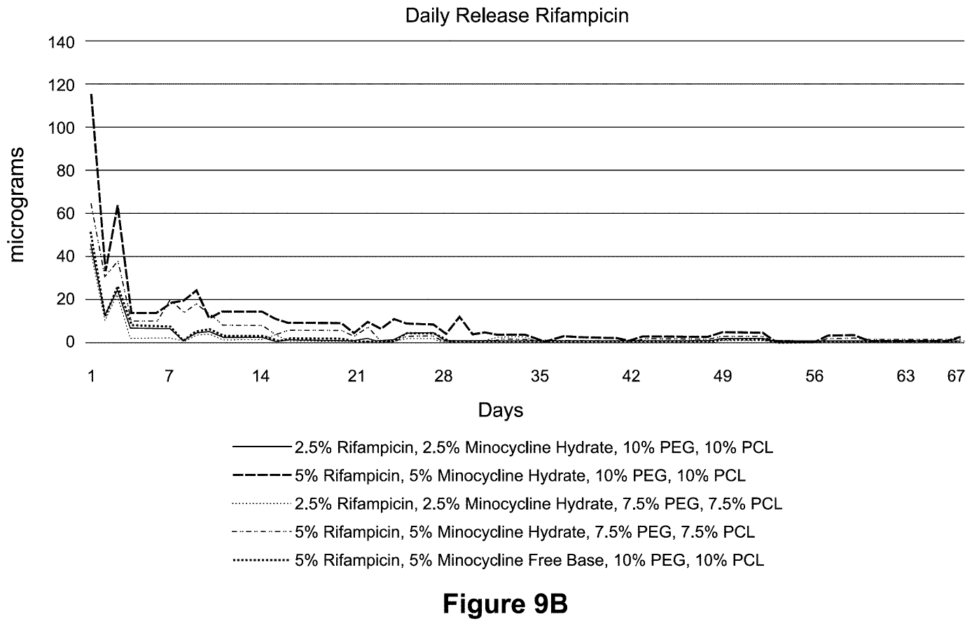

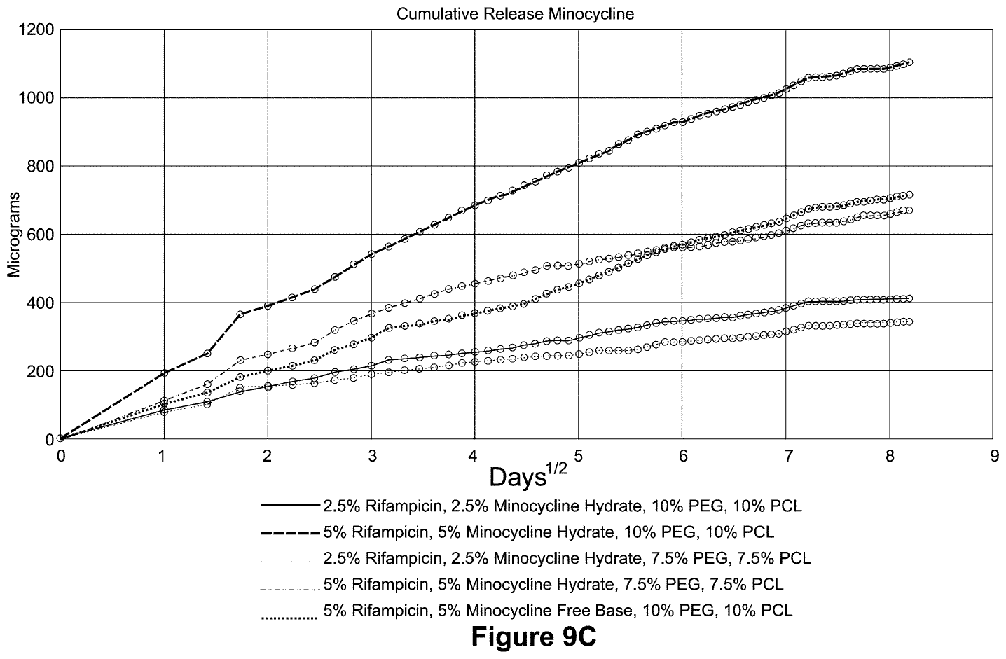

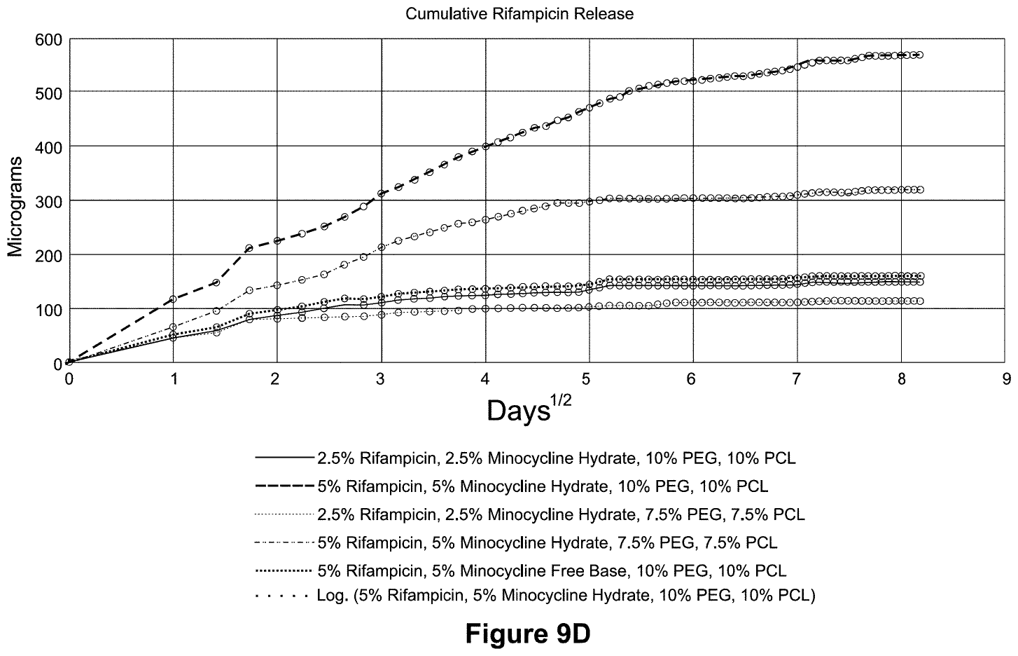

We performed another series of experiments also using Blend 1 through Blend 5. In this new set of experiments, in contrast to the just-presented results, the bath into which the drug is released is Phosphate Buffered Saline, having a pH of 6.7, at 37° C., stirred, and the data extends to a longer period of time. Stirring and buffering at the higher temperature better mimics conditions in the human body, which makes the release study a better analogue to its possible applications. Referring now to FIG. 9A, there is shown a similar plot of Incremental Daily Release of Minocycline and in FIG. 9B for Rifampin. Also, FIGS. 9C and 9D show cumulative release of each drug, corresponding to FIGS. 9A and 9B.

Release data includes data from five blends having varying concentrations of API and varying concentrations of PEG/PCL. This demonstrates effects of polymer additives (composition having 10% concentration each of PEG and PCL has more release than composition having 7.5% concentration each of PEG and PCL). This also demonstrates the effects of increasing Minocycline content from 2.5% to 5% (Release from a composition containing 5% minocycline is greater than release from a composition containing 2.5% minocycline). The data is reported in Table 7.

TABLE 7

| | | | | | | Linearized | Total | Linearized | |
|---|---|---|---|---|---|---|---|---|---|
| | | | | | | Release | Release | Release | Total |
| | | | | | | rate | at day | rate | Release |
| | | | | | | MINO | 30 | RIF | at day |
| Blend | EVA | PEG | PCL | RIF | MINO | [ug/day^{1/2}] | MINO | [ug/day^{1/2}] | 30 RIF |
| 1 | 75% | 10% | 10% | 2.5% | 2.5% | 45.05 | 321 | 11.74 | 141 |
| 2 | 70% | 10% | 10% | 5% | 5% | 124.25 | 874 | 61.07 | 506 |
| 3 | 80% | 7.5% | 7.5% | 2.5% | 2.5% | 34.57 | 258 | 7.79 | 104 |
| 4 | 75% | 7.5% | 7.5% | 5% | 5% | 69.13 | 535 | 29.48 | 302 |
| 5 | 70% | 10% | 10% | 5% | 5%* | 88.22 | 514 | 12.16 | 153 |

From the cumulative release graphs, it can be seen that the change of environmental conditions increased the overall release from polymer samples roughly fivefold. This indicates that conditions of the human body will increase release from the polymer over time. This study also reconfirms the trends from the first release study, namely: the benefit of increasing API concentration, the benefit of 10% polymer additive over 7.5% polymer additive, and the indication of degradation of free base minocycline (while still indicating passable release).

### Example 5

A further series of experiments was performed using Blend 6 through Blend 15. The choice of compositions for Blend 6 through Blend 15 used some of the optimization conclusions just described that were learned from experiments using Blend 1 through Blend 5. Table 8 includes the compositions of Blend 6 through Blend 15.

TABLE 8

| | | | | | | Linearized | Total | Linearized | Total |
|---|---|---|---|---|---|---|---|---|---|
| | | | | | | Release | Release at | Release | Release at |
| | | | | | | rate MINO | day 30 | rate RIF | day 30 |
| Blend | EVA | PEG | PCL | RIF | MINO | [ug/day^{1/2}] | MINO[ug] | [ug/day^{1/2}] | RIF[ug] |
| 6 | 75% | 10% | 10% | 5% | 0% | — | — | 28.53 | 143 |
| 7 | 75% | 10% | 10% | 0% | 5% | 1.62 | 59 | — | — |
| 8 | 70% | 10% | 10% | 5% | 5% | 131.49 | 663 | 73.40 | 378 |
| 9 | 60% | 10% | 10% | 10% | 10% | 409.05 | 2097 | 195.93 | 1055 |
| 10 | 40% | 10% | 10% | 20% | 20% | 399.13 | 5992 | 559.74 | 5415 |
| 11 | 80% | 10% | 0% | 5% | 5% | 52.02 | 350 | 51.11 | 272 |
| 12 | 80% | 0% | 10% | 5% | 5% | 31.96 | 141 | 40.54 | 187 |
| 13 | 90% | 0% | 0% | 5% | 5% | 24.09 | 160 | 27.15 | 148 |
| 14 | 40% | 10% | 10% | 20%** | 20% | 400.86 | 6972 | 475.00 | 5073 |
| 15 | 70% | 10% | 10% | 5%** | 5% | 138.13 | 694 | 55.07 | 315 |

### Example 6: Cumulative Release of Both Drugs from a Specific Blend (Blend 9)

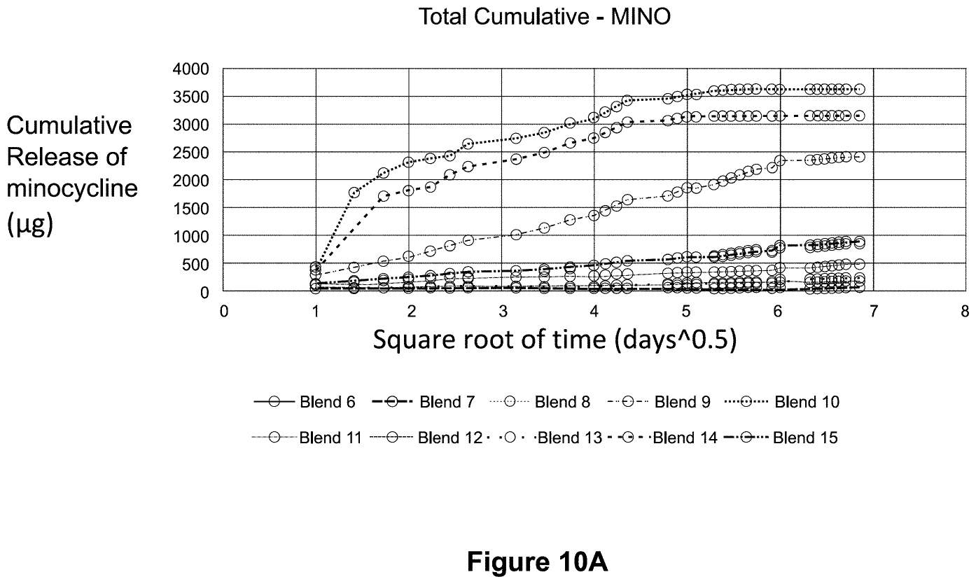

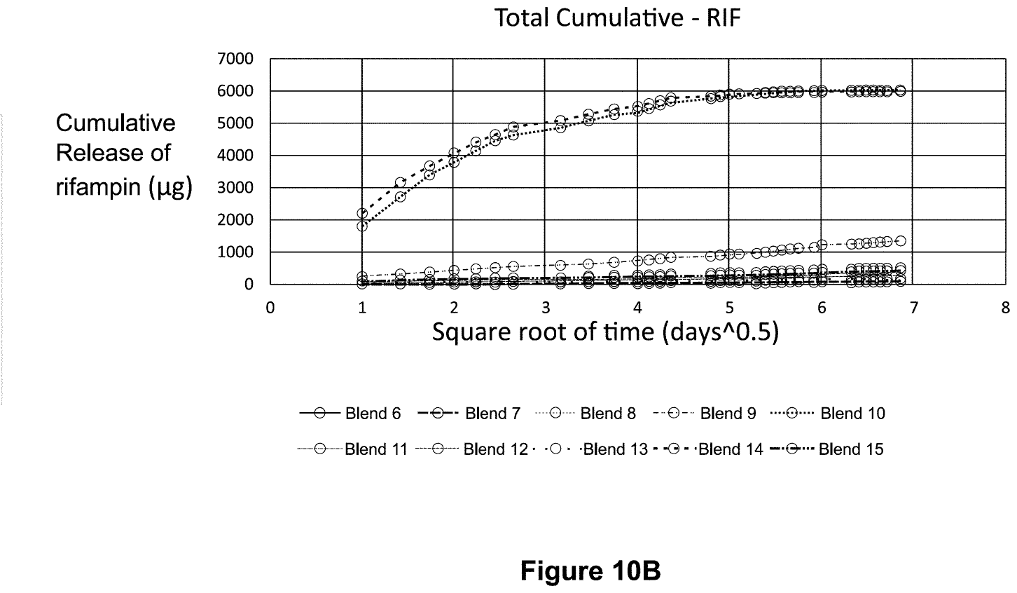

FIG. 10A shows the cumulative release of minocycline from each blend, in terms of the cumulative amount of minocycline released. FIG. 10B shows the cumulative release of rifampin from each blend, in terms of the cumulative amount of rifampin released. It can be seen that except for Blends 10 and 14, the release is approximately zero order release, which can be seen because when cumulative release is plotted against square root of time there is a generally straight line generated.

It can be seen that Blend 9 was again the blend with the most consistent release as desired for present purposes, having a relatively high release and a substantially straight profile in this form of plot. Also, it has a larger release (which is desirable for present purposes) than most of the other compositions.

Blends (Blends 10 and 14), which contain a 20% concentration of drug, are observed to have a cumulative release profile that does not so much exhibit a linear relationship in the above form of plot. Instead, the cumulative release profile is fairly high in the early portion of this graph and then somewhat flattens out in the later part of the graph. It is believed that those two compositions somewhat run out of drug in the later part of the experiment, which results in the flattening of the curve that is visible after around day 26.

These graphs also clearly illustrate the benefit of having an additional release-modifying additive in the form of PCL. The release from Blend 8 is well above the release from Blend 11. An important difference in composition is that Blend 8 contains PCL while Blend 11 does not contain PCL. This demonstrates that there is a benefit of including PCL in the blend of polymers.

In FIG. 10B, the release of rifampin is plotted from the same blends as were plotted in FIG. 10A. It can be seen that there are the same trends and conclusions as Minocycline, with a similar graph following the same general trends as were observed in FIG. 10A. Again, Blend 9 gives the largest release among the blends that show a generally linear relationship between cumulative release and square root of time. The only blends that show a larger cumulative release are Blends 10 and 14, but those releases flatten out before the end of the period of acquisition of data.

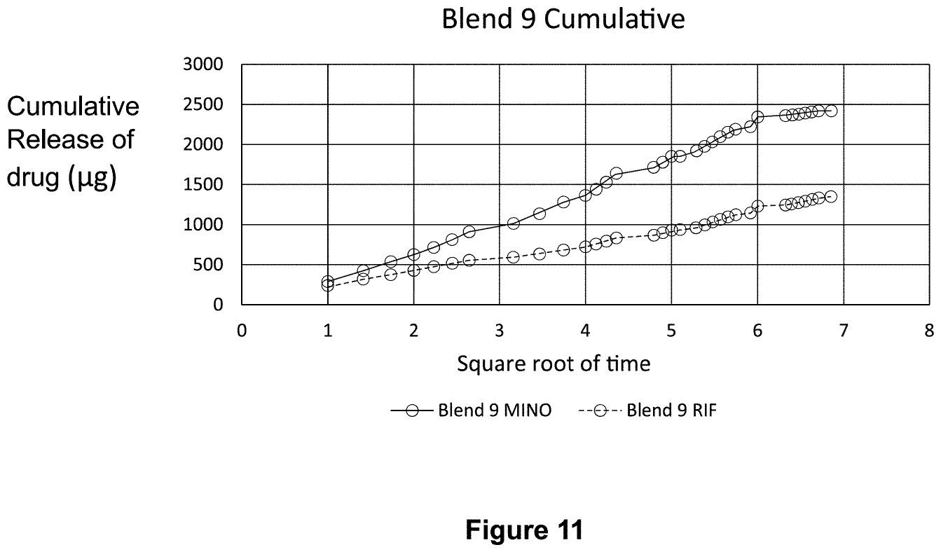

As was the case in the experiments presented herein for which both drugs were present in the composition, the concentration of minocycline and the concentration of rifampin were equal in the formulation. For the composition of Blend 9, the graph shows both minocycline release and rifampin release (cumulative release) graphed on the same plot. Minocycline release is plotted in blue, and rifampicin release is plotted in orange. This illustrates that the two drugs show similar trends trend although there is some difference in the magnitudes of the releases. For example, at most time points, the cumulative release of minocycline is approximately double the cumulative release of rifampin. See FIG. 11.

We might expect rifampin to release at a slower rate than minocycline because rifampin is significantly more hydrophobic than minocycline, as well as having an aqueous solubility that is smaller by more than an order of magnitude. This experimental result shows that while rifampin release is somewhat slower than minocycline release, it is not as much slower as might have been expected, and there is still a useful amount of release of both drugs.

### Example 7: Effect of Polymer Additives

An experiment was conducted to investigate release properties of Elvax alone and of Elvax with either or both of two additives, PEG and PCL. For this experiment, the concentration of each drug in the formulation was 5%. The concentration of Elvax was adjusted to accommodate the concentrations of the various other substances. Release of each drug is measured individually. The graph below shows the measurements for minocycline, and the graph after that shows similar measurements for rifampicin.

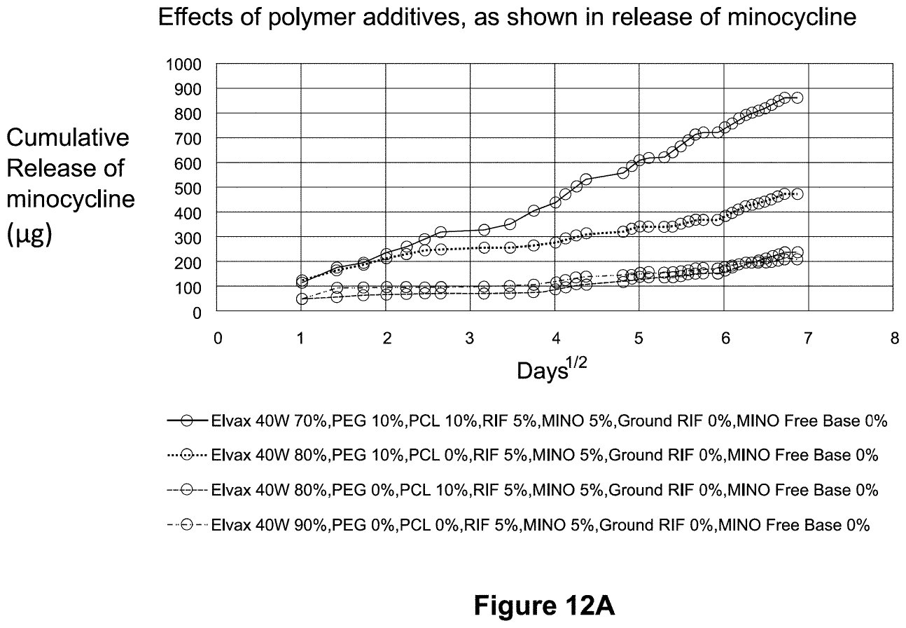

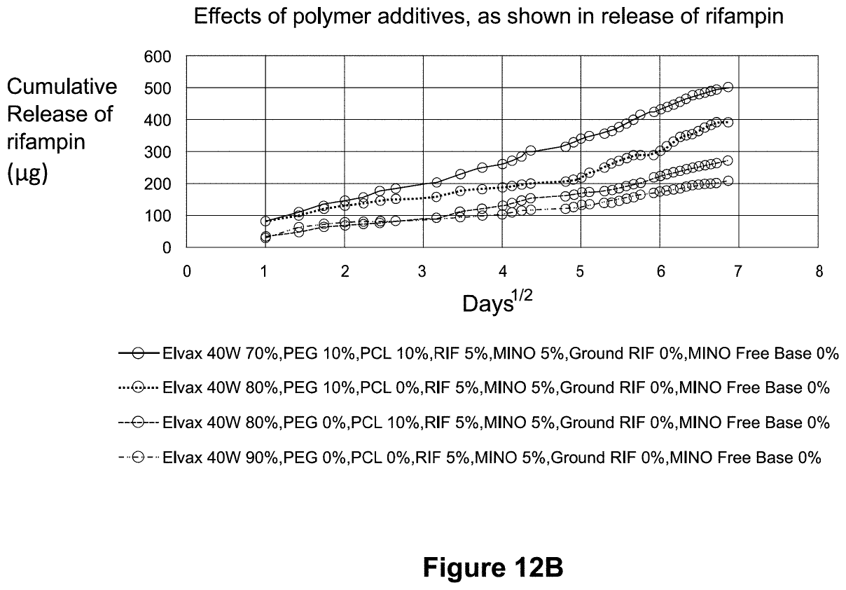

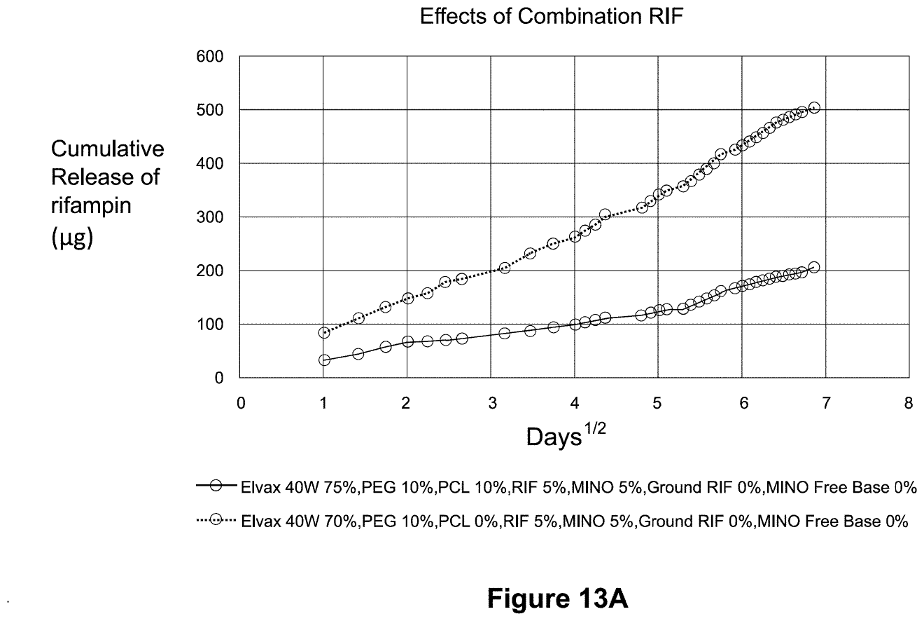

The results of this experiment seem to indicate that the release from Elvax alone is small, and the release with a 10% concentration of PCL added to the Elvax is almost unchanged from the Elvax-only release. If 10% PEG is added to the Elvax, the release slightly more than doubles compared to the Elvax-only release. If the additives to Elvax include both PEG at 10% concentration and PCL at 10% concentration, the release is still further increased. For the PEG+PCL combination, the cumulative release approximately doubles again and is about four times the Elvax-only release. This suggests that including both PEG and PCL as additives is further helpful in achieving release and achieves the best result. The data is summarized in Table 9. See FIGS. 12A and 12B.

TABLE 9

| | | | | | | Linearized | Total | Linearized | Total |
|---|---|---|---|---|---|---|---|---|---|
| | | | | | | Release rate | Release at | Release rate | Release at |
| | | | | | | MINO | day 30 | RIF | day 30 |
| Blend | EVA | PEG | PCL | RIF | MINO | [ug/day^{1/2}] | MINO | [ug/day^{1/2}] | RIF |
| 8 | 70% | 10% | 10% | 5% | 5% | 131.49 | 663 | 73.40 | 378 |
| 11 | 80% | 10% | 0% | 5% | 5% | 52.02 | 350 | 51.11 | 272 |
| 12 | 80% | 0% | 10% | 5% | 5% | 31.96 | 141 | 40.54 | 187 |
| 13 | 90% | 0% | 0% | 5% | 5% | 24.09 | 160 | 27.15 | 148 |

### Example 8: Release of One Drug in the Presence or Absence of the Other Drug, for Otherwise Identical Formulations

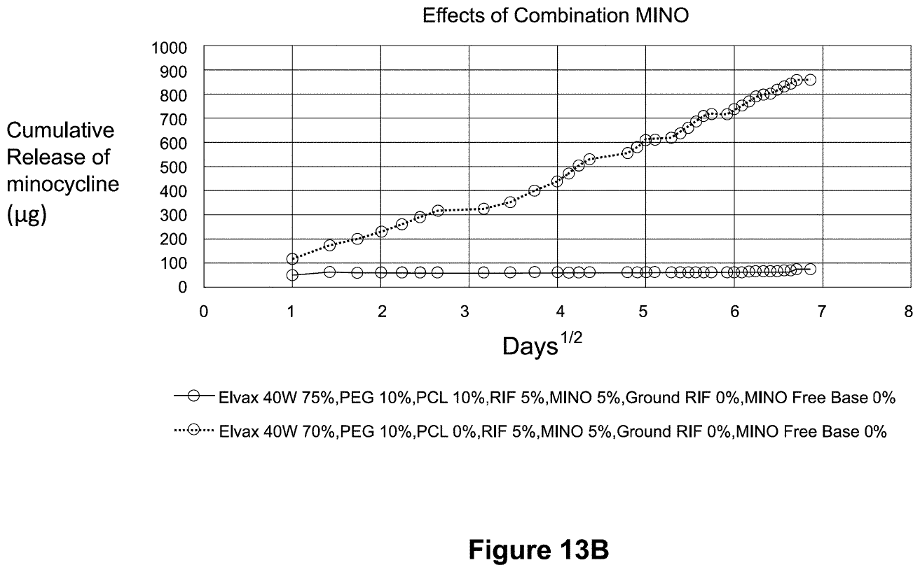

An experiment was conducted measuring the release of rifampicin in two different circumstances that otherwise were identical. In one instance, rifampin and minocycline were present in equal concentrations. In the other instance, rifampin was present alone without any minocycline. Both formulations included a 10% concentration of PEG and a 10% concentration of PCL, with the Elvax concentration being adjusted in accordance with the presence or absence of minocycline. It can be seen that in the presence of minocycline, the cumulative release of rifampin is greater, an increase by a factor of about 2.5. This suggests some sort of synergy involved in the release of rifampin, when minocycline is present compared to when minocycline is absent. Release of Rifampicin in the presence or absence of minocycline in the composition. The data is summarized in Table 10. See FIGS. 13A and 13B.

TABLE 10

| | | | | | | Linearized | Total |
|---|---|---|---|---|---|---|---|
| | | | | | | Release | Release |
| | | | | | | rate RIF | at day 30 |
| Blend | EVA | PEG | PCL | RIF | MINO | [ug/day^{1/2}] | RIF[ug] |
| 6 | 75% | 10% | 10% | 5% | 0% | 28.53 | 143 |
| 8 | 70% | 10% | 10% | 5% | 5% | 73.40 | 378 |

A similar experiment was conducted measuring the release of minocycline in two different circumstances that otherwise were identical. In one instance, minocycline and rifampin were present in equal concentrations. In the other instance, minocycline was present alone without any rifampin. Both formulations included a 10% concentration of PEG and a 10% concentration of PCL, with the Elvax concentration being adjusted in accordance with the presence or absence of rifampin. It can be seen that in the presence of rifampin, the cumulative release of minocycline is greater, an increase by a factor of about 8. This suggests some sort of synergy involved in the release of minocycline when rifampin is present, compared to when minocycline is absent. The data is summarized in Table 11.

TABLE 11

| | | | | | | Linearized | Total |
|---|---|---|---|---|---|---|---|
| | | | | | | Release | Release at |
| | | | | | | rate MINO | day 30 |
| Blend | EVA | PEG | PCL | RIF | MINO | [ug/day^{1/2}] | MINO[ug] |
| 7 | 75% | 10% | 10% | 0% | 5% | 1.62 | 59 |
| 8 | 70% | 10% | 10% | 5% | 5% | 131.49 | 663 |

### Example 9: Effect of Concentration on Release of Minocycline and Rifampicin

It also is possible to investigate how the release of drug depends on the absolute magnitude of the concentration of drug in the composition. In this experiment, at the time of manufacture, the concentration of minocycline in the composition and the concentration of rifampin in the composition were always equal to each other. The compositions tested here had an individual drug concentration of either 5% or 10% or 20%. (With the individual drug concentrations being 5% or 10% or 20%, the total drug concentration was 10% or 20% or 40%.) In these various formulations, the concentration of PEG was held constant at 10% and the concentration of PCL was held constant at 10%. The concentration of Elvax 40 was what was varied to compensate for the varying amounts of drug.

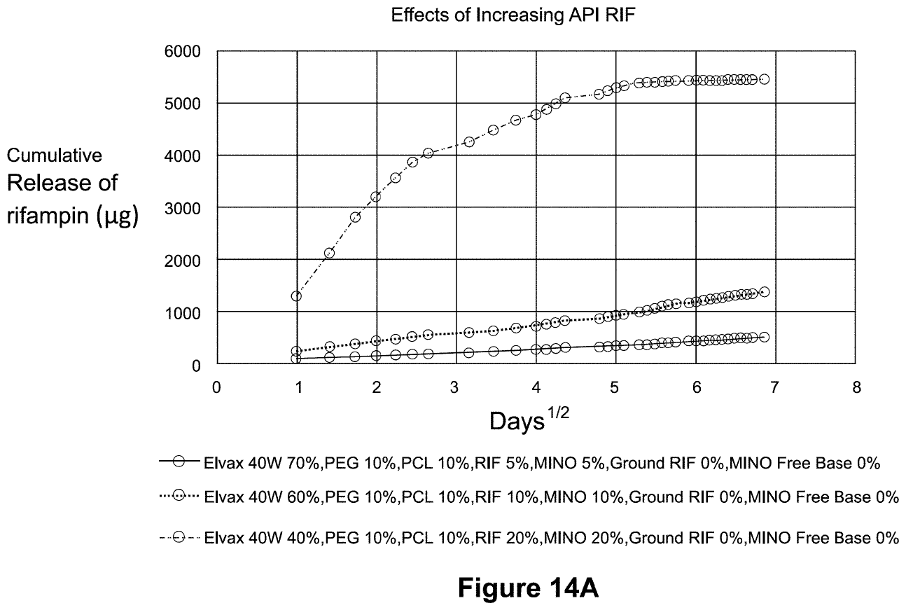

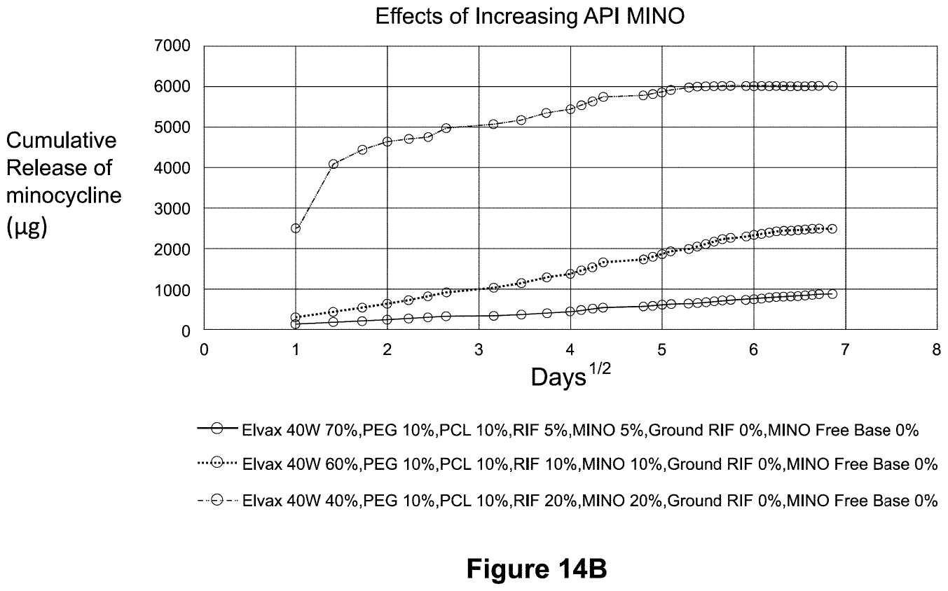

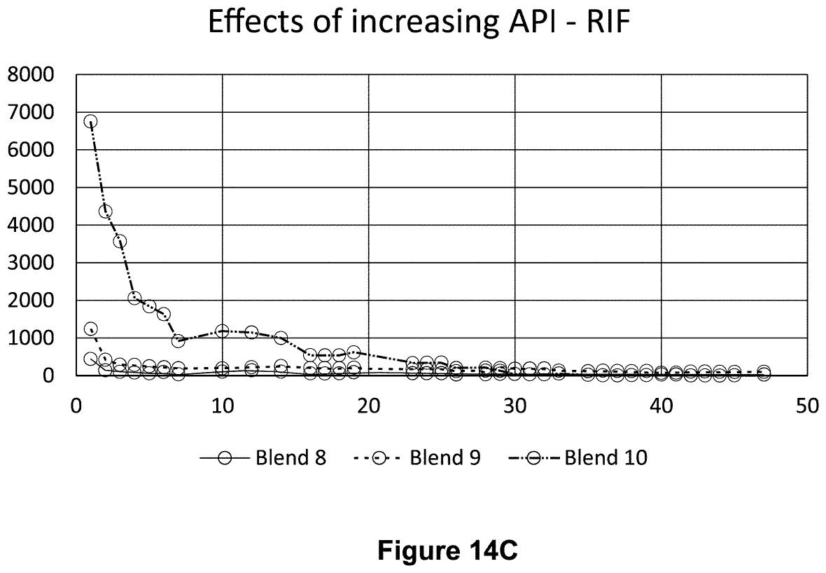

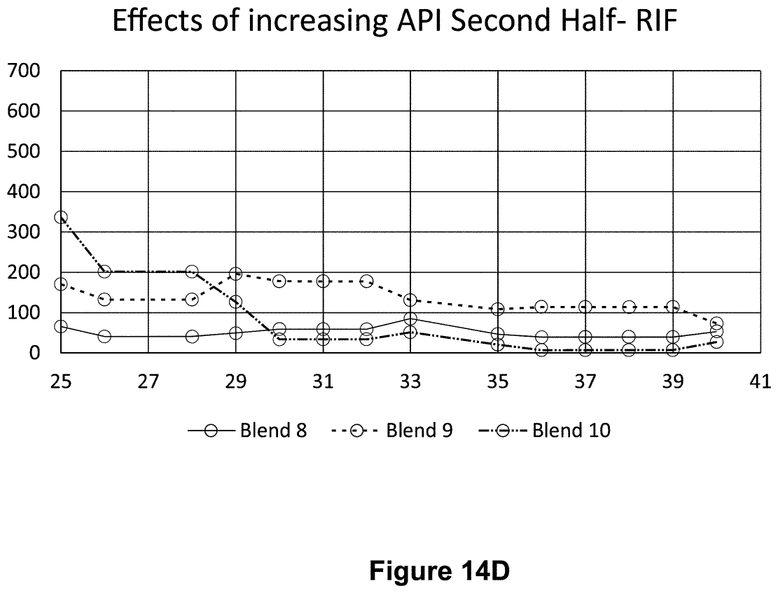

FIG. 14A shows the release of rifampin (while of course minocycline was also released). FIG. 14B shows the release of minocycline (while of course rifampin was also released). In general, as would be expected, the higher drug concentration increased the cumulative release and release rate. For each drug, the release rate at 10% concentration of the drug was larger than the release rate at 5% concentration of the drug. It can be seen that when the individual drug concentration was 20%, the release rate in the early part of the experiment was markedly stronger, but toward the end of the experiment the release curve became flat, i.e., toward the end of the experiment there was no further release. For the smaller individual drug concentrations (5% or 10%), drug was still being released even at the end of the experiment. The data is summarized in Table 12.

TABLE 12

| | | | | | | Linearized | Total | Linearized | Total |
|---|---|---|---|---|---|---|---|---|---|
| | | | | | | Release rate | Release at | Release rate | Release at |
| | | | | | | MINO | day 30 | RIF | day 30 |
| Blend | EVA | PEG | PCL | RIF | MINO | [ug/day1/2] | MINO | [ug/day1/2] | RIF |
| 8 | 70% | 10% | 10% | 5% | 5% | 131.49 | 663 | 73.40 | 378 |
| 9 | 60% | 10% | 10% | 10% | 10% | 409.05 | 2097 | 195.93 | 1055 |
| 10 | 40% | 10% | 10% | 20% | 20% | 399.13 | 5992 | 559.74 | 5415 |

This phenomenon can also be seen in FIG. 14C which shows incremental release of rifampin and FIG. 14D zooms in on the relevant portion of the graph showing depletion of 20% API blend. Release rate reduces below that of the lower API blends (5 and 10%) after day 26 indicating that the 20% API blend is less suitable for long term release and more suitable for short term release. The data is reported in Table 13.

TABLE 13

| | | | | | | Linearized | Total | Linearized | Total |
|---|---|---|---|---|---|---|---|---|---|
| | | | | | | Release rate | Release at | Release rate | Release |
| | | | | | | MINO | day 30 | RIF | at day 30 |
| Blend | EVA | PEG | PCL | RIF | MINO | [ug/day1/2] | MINO | [ug/day1/2] | RIF[ug] |
| 8 | 70% | 10% | 10% | 5% | 5% | 131.49 | 663 | 73.40 | 378 |
| 9 | 60% | 10% | 10% | 10% | 10% | 409.05 | 2097 | 195.93 | 1055 |
| 10 | 40% | 10% | 10% | 20% | 20% | 399.13 | 5992 | 559.74 | 5415 |

### Example 10: Effect of Grinding Rifampicin

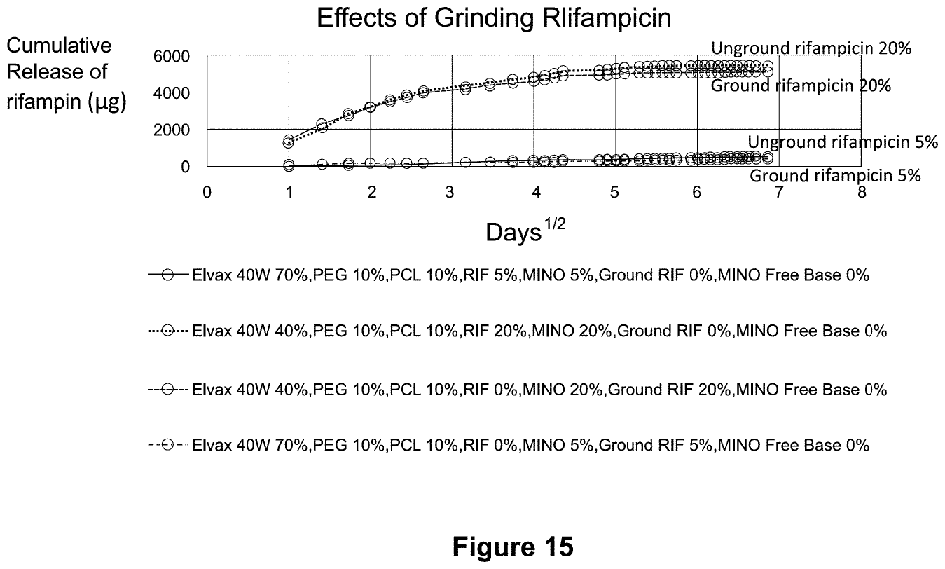

Rifampicin is obtained commercially in the form of flat crystalline particles of typical dimension 70-100 microns. For some formulations, the rifampin is mixed into the blend in that as-purchased crystalline form. For other formulations, the rifampin is ground into a smaller particle size before being mixed into the blend. Grinding was performed using a planetary mill, Pulverisette 7, made by Fritsch International (Pittsboro, N.C.). This is done to see if there is an effect of particle size on drug release characteristics. The ground particles were smaller than the unground particles, and there seemed to be little effect. The comparison is shown in FIG. 15.

In FIG. 15, two of the plots are for a rifampin concentration of 5% (together with an equal concentration of minocycline). These two plots, one for as-purchased crystalline rifampin and the other for ground rifampin, almost overlay each other.

In FIG. 15, the other two plots are for a rifampin concentration of 20% (together with an equal concentration of minocycline). Similarly, these two plots, one for as-purchased crystalline rifampin and the other for ground rifampin, almost overlay each other.

From this data. It appears that there is not much effect on the release profile according to whether the rifampin particles are ground or not. The data is reported in Table 14.

TABLE 14

| | | | | | | Linearized | Total | Linearized | Total |
|---|---|---|---|---|---|---|---|---|---|
| | | | | | | Release rate | Release at | Release rate | Release at |
| | | | | | | MINO | day 30 | RIF | day 30 |
| Blend | EVA | PEG | PCL | RIF | MINO | [ug/day1/2] | MINO | [ug/day1/2] | RIF |
| 8 | 70% | 10% | 10% | 5% | 5% | 131.49 | 663 | 73.40 | 378 |
| 10 | 40% | 10% | 10% | 20% | 20% | 399.13 | 5992 | 559.74 | 5415 |
| 14 | 40% | 10% | 10% | 20%** | 20% | 400.86 | 6972 | 475.00 | 5073 |
| 15 | 70% | 10% | 10% | 5%** | 5% | 138.13 | 694 | 55.07 | 315 |

### Example 11: Minimum Inhibitory Concentration

Cumulative release graphs, as have been shown in some previous Examples, are well suited for showing trends when composition was varied, and this was useful in various Examples. On the other hand, if we are concerned with demonstrating effectiveness against bacteria, we want to show that every single day sufficient drug is released to achieve effectiveness against bacteria. This favors display of data essentially as release rate, which is plotted herein as incremental (daily) release rate, with the measurement interval usually being one day.

In connection with displaying drug release data, it is helpful to realize that in a clinical situation, there is some characteristic volume of tissue, near the fixator pin or other medical device, into which the drug is released. This is in recognition of the fact that drug released into tissue or interstitial fluid will have some tendency to disperse or travel within that tissue or interstitial fluid. In regard to the latter, it can be appreciated that there is not likely to be a sharp boundary between where drug is present and where drug is absent. Nevertheless, in order to obtain a rough approximate idea of effectiveness, it is useful is to assign the released drug to an assumed space. Herein, such space is referred to as a Pin Volume Estimate. The purpose of the pin volume estimate is to contextualize the results of In vitro release testing (IVRT). What the estimate does is normalize for the size of the coupon and the volume of the experimental bath, which are both different from the environment in the human tissue, but can be related to the environment in the human tissue. In applications of interest such as the region around a fixator pin or catheter, a typical geometry is likely to be cylindrical or annular.

Pin volume parameters used in further calculations herein are shown in Table 15.

TABLE 15

| Parameter | Value | Unit |
|---|---|---|
| Polymer Length | 40 | mm |
| Pin Diameter | 1 | mm |
| Polymer Thickness | 2 | mm |
| Distance into Muscle | 2 | mm |
| Volume Around Polymer | 1.26 | cm3 |

Expected concentration = (Total Micrograms of drug released per gram coupon * Theoretical weight of polymer on pin)/volume around pin

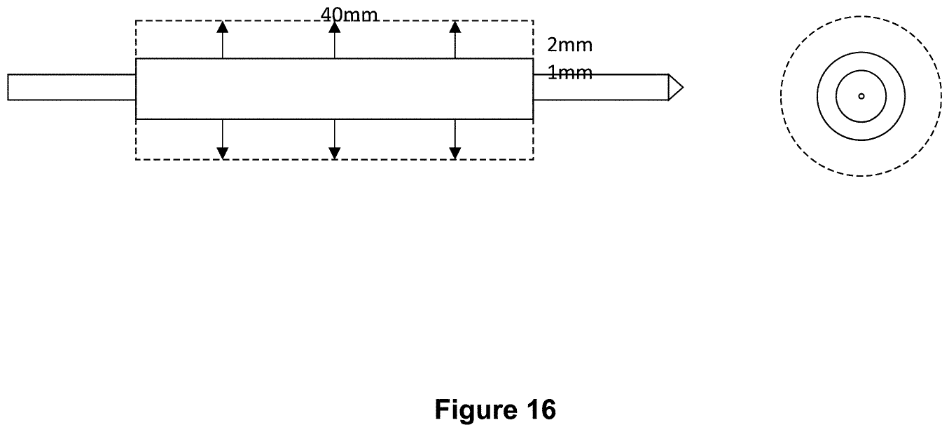

The mass of the pin is calculated using the assumed dimensions of a concentric cylinder as shown in FIG. 16 illustrating fixator pin, and the density of the polymer. The volume a round pin is from dimensions of an annular region concentric with the pin, with an assumed distance into the human tissue. Herein, the estimated distance used is 2 mm. This number is assumed as being a reasonable representative distance for the drug to penetrate into the adjacent tissue and interstitial fluid. The data presented here, which is for Blend 9, uses this pin volume estimate to calculate whether the MIC is achieved.

### Assumptions:

Total exchange over the course of a day
   We change the media in the lab for the IVRT every day. In the tissue, this means the drug is totally removed over 24 hours
   This is not the best assumption possible since the IVRT experiment is done batchwise rather than being continuously removed as in actual tissue, but there is likely to be drug accumulation in the human tissue where we do not have that situation, and keep a perfect sink in the IVRT. This tends to err on the side of less available drug in the human tissue.
Sink conditions met
   Sink conditions in this IVRT mean that we are 10× above the saturation concentration of the drugs
   Sink conditions are not uncommon during the use of controlled release devices and are frequently used in IVRTs which model controlled release from polymers. Again, this errs on the side of less available drug in the interstitial fluid of the human tissue, because since if the human tissue saturates, then more of the drug remains in the polymer composition over time allowing for longer periods of high release than are seen in the study.

### Normalization by Minimum Inhibitory Concentration (MIC)

Even when distribution of the released drug in an assumed pin volume is taken into consideration, there remains yet another factor in assessing how effective that drug release is in particular situations. One such factor is how much drug is required in order to be effective against a particular species of bacteria. This varies for different combinations of drug and species of bacteria. This is usually expressed as Minimum Inhibitory Concentration, which is the minimum concentration of a particular drug that is needed to be effective against a particular species of bacteria.

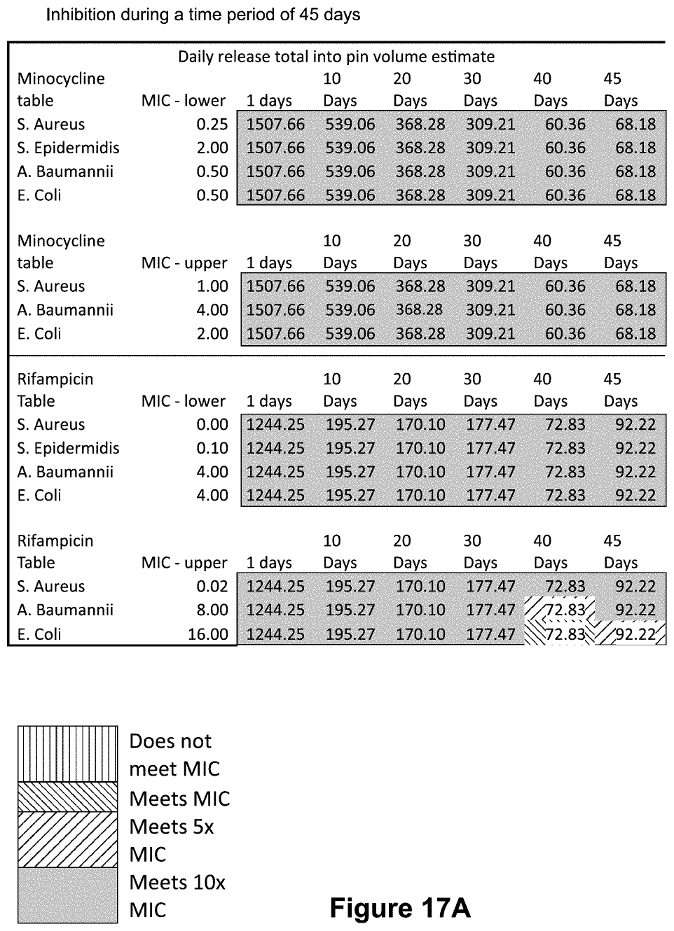

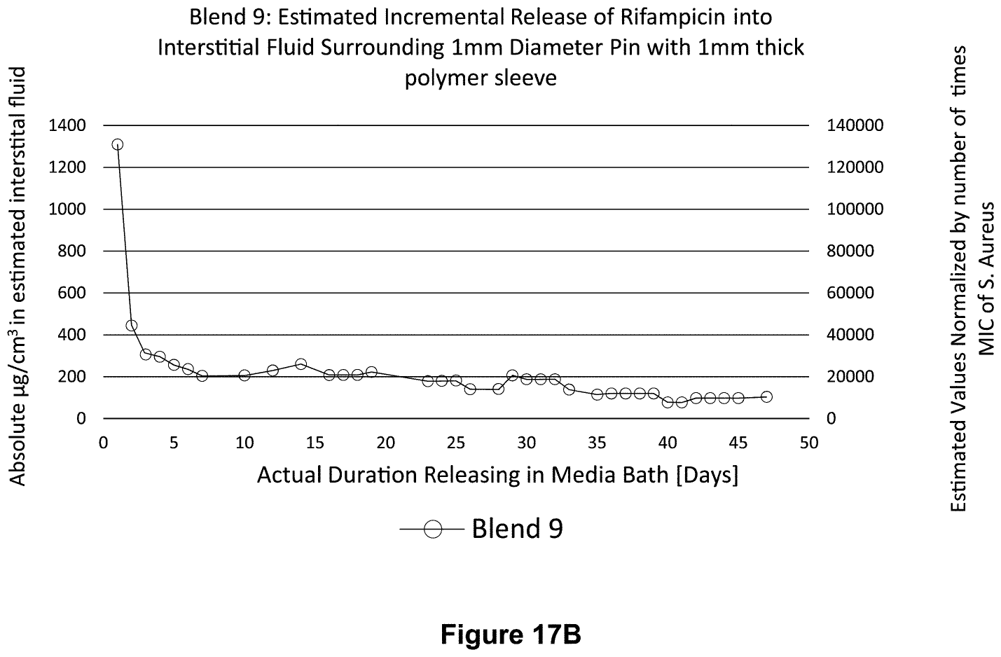

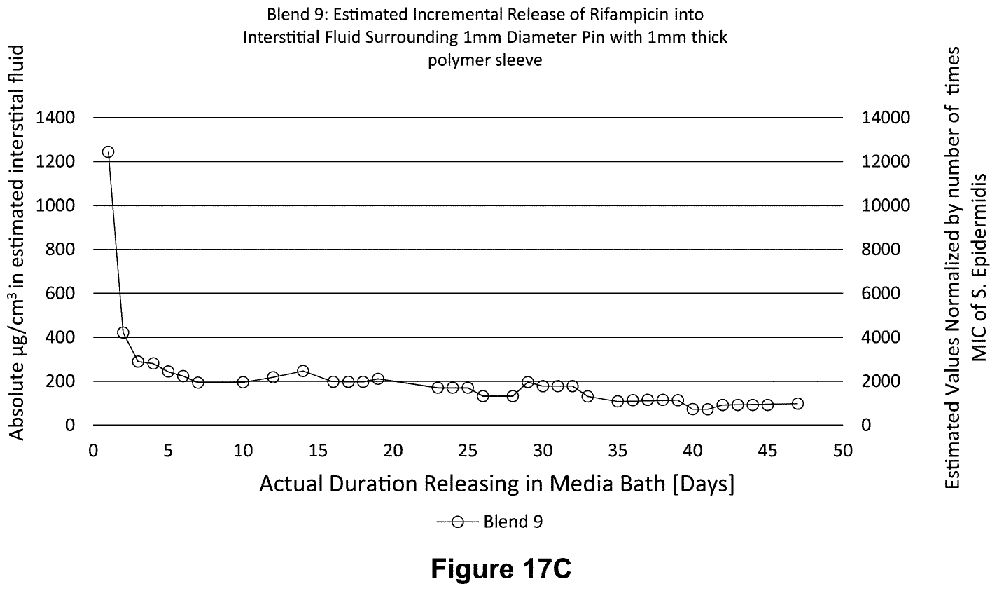

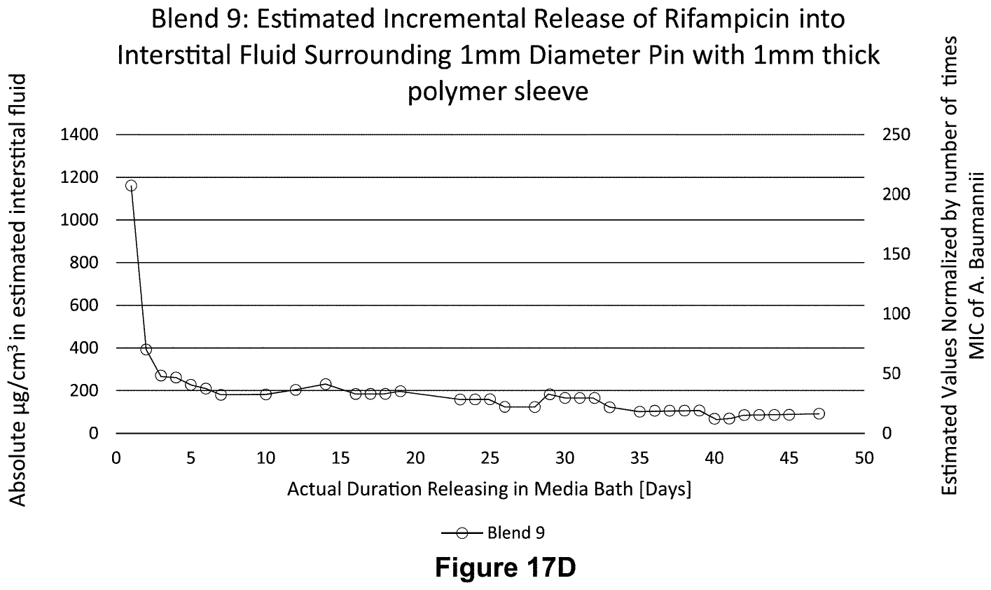

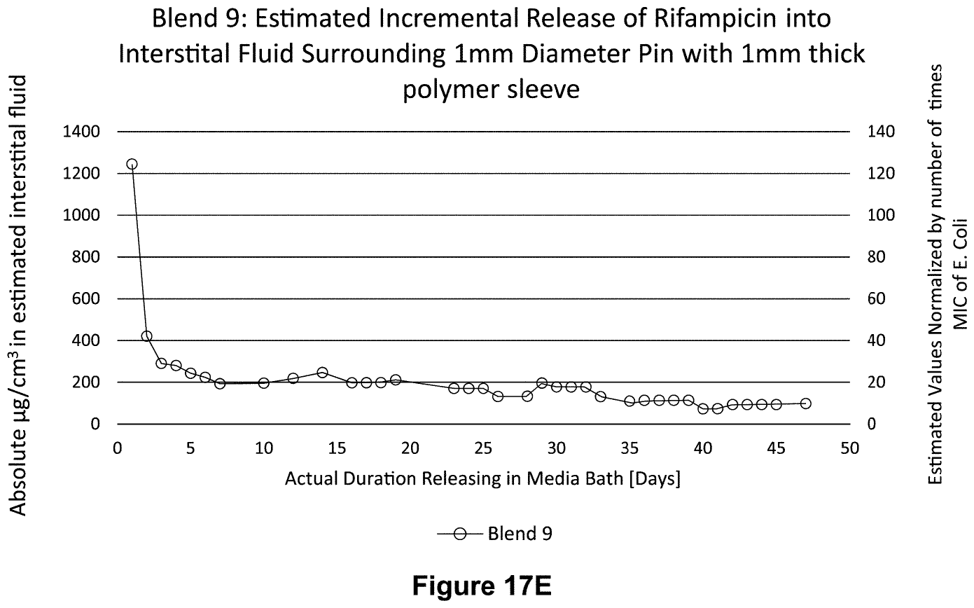

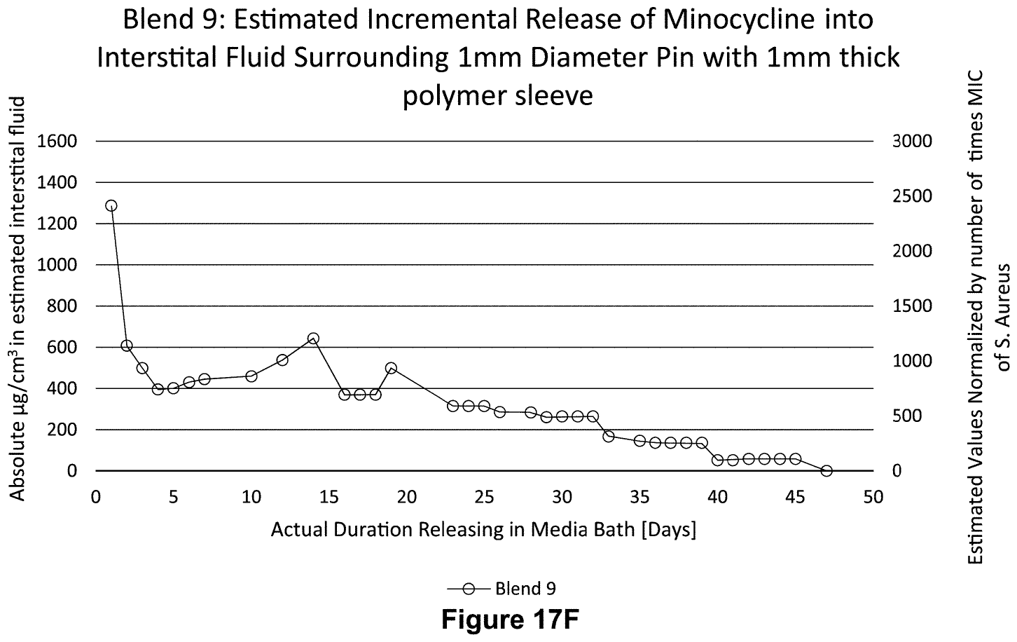

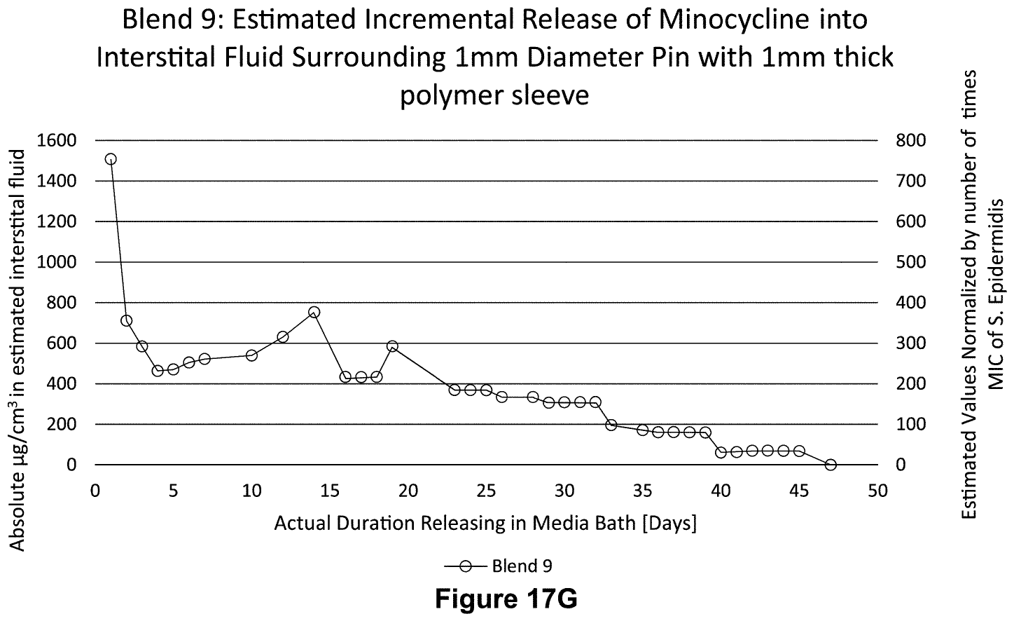

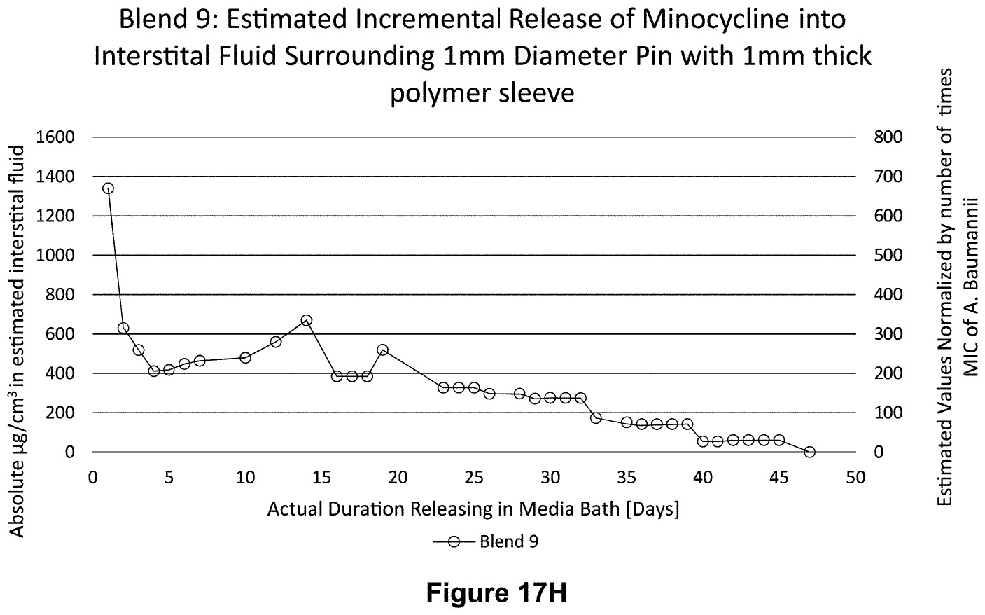

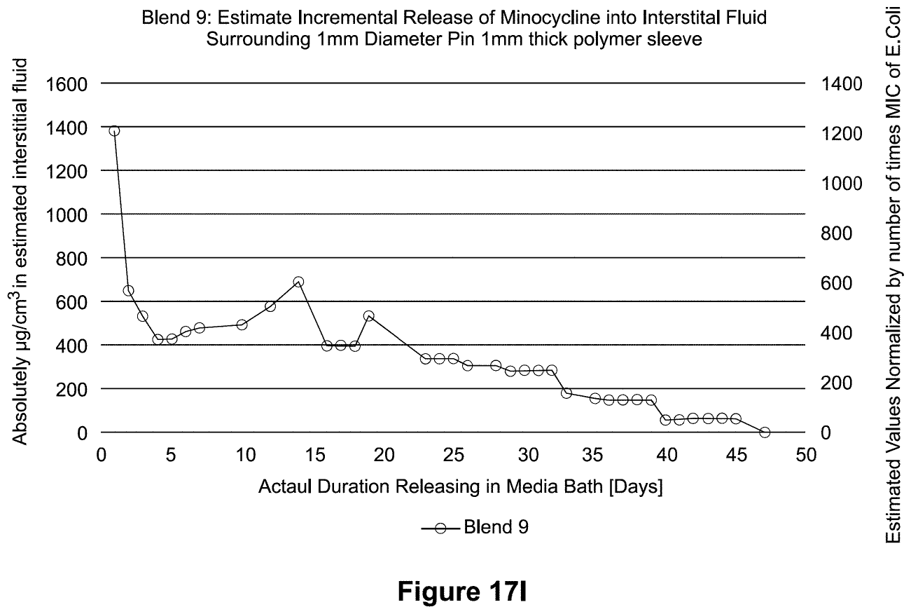

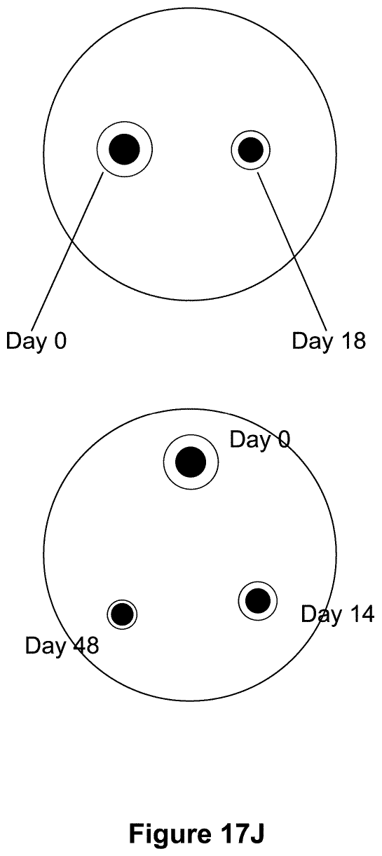

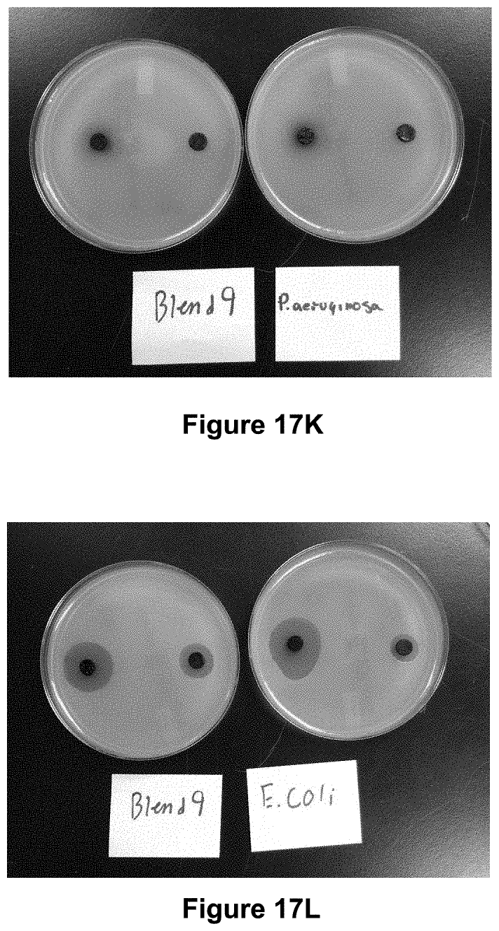

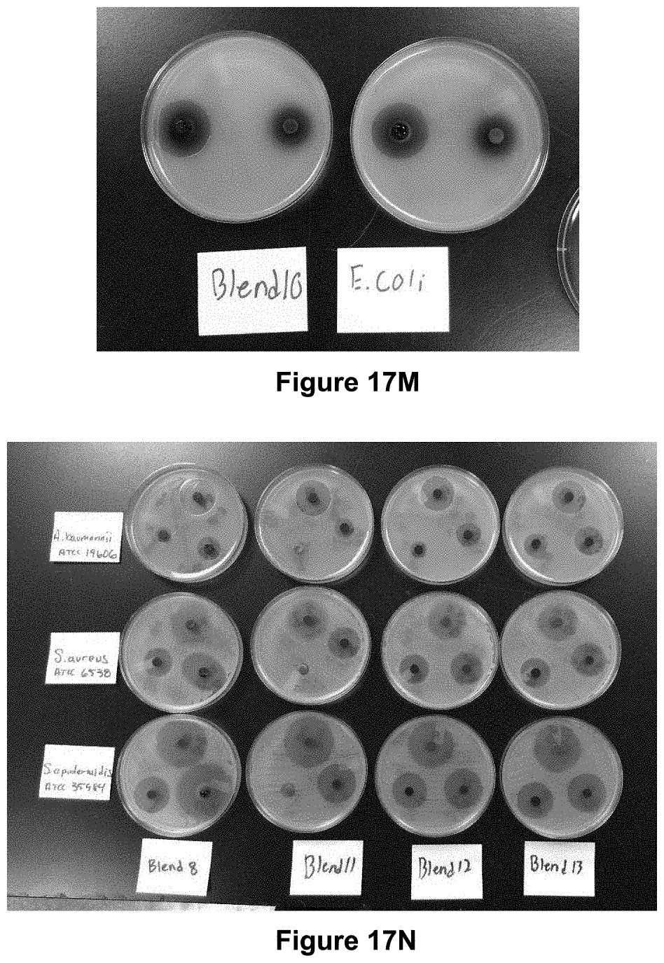

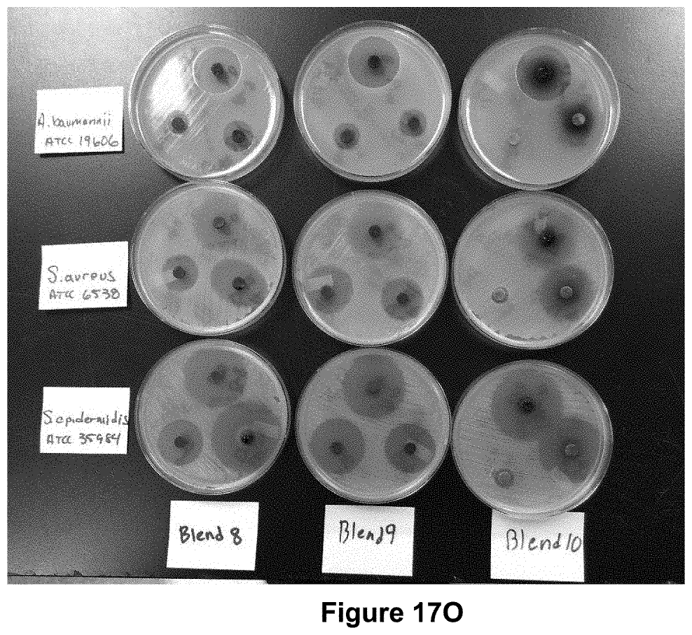

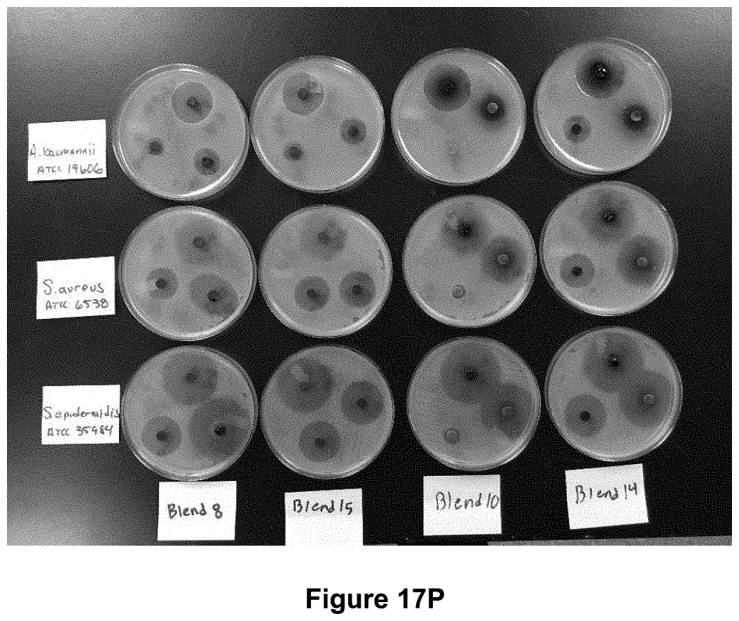

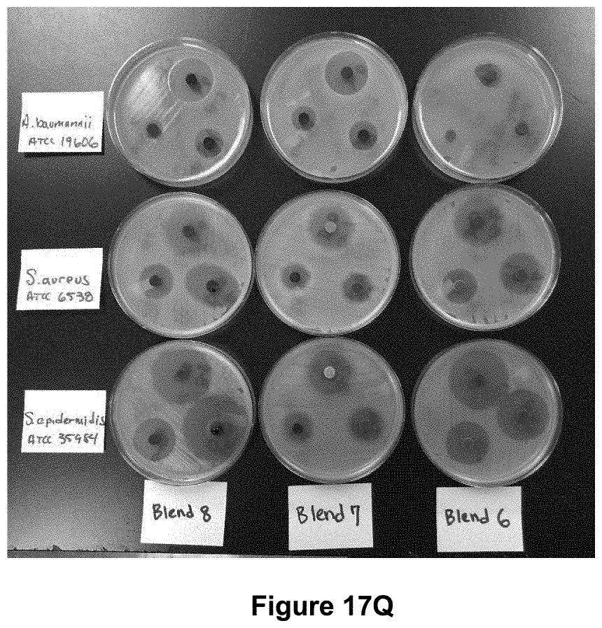

For present calculations, the values of MIC used for various bacteria are given in Table 16 in units of micrograms per ml. This is averaged between the high and low of the MIC shown on FIG. 17A.

TABLE 16

| Bacteria | Minocycline | Rifampicin |
|---|---|---|
| S. Aureus | 0.625 | 0.0095 |
| S. Epidermidis | 2 | 0.1 |
| A. Baumanii | 2.25 | 6 |
| E. Coli | 1.25 | 10 |

It can further be appreciated that while achieving a concentration of exactly the MIC in human tissue would have some merit, in a practical sense it would be desirable to exceed the MIC by some factor. For present purposes, it is considered that a desirable factor compared to the MIC is a factor of approximately 10.

In FIG. 17A, for Blend 9, there are illustrated the MICs we found for each drug relating to important organisms. We used the expected concentration in the volume around the pin to gauge up to 45 days if we were meeting the desired criterion of 10× the MIC. The graphs in “Graphs normalized by the pin volume estimate” use these same numbers showing the estimated concentration for every day of the study, as well as how many times the MIC. This is with each drug and each organism, the MIC used in these graphs are an average between the high and low. Blend 9 data is used because we consider it to be the currently preferred polymer blend for long duration release. Blend 9 contains a 10% concentration of each drug. (In other experiments, it was found that Blend 10, which contains a 20% concentration of each drug, gives higher drug release values early in the experiment but after day 25 it has significantly lower values so it is worse for long term applications although for shorter term applications it might be better. Accordingly, for the present study, Blend 9 was used.) It can be seen that even for the upper bounds of minocycline and rifampicin, our expected concentrations of the drugs exceed 10× the MIC for at least one drug present.

These are basically graphs of incremental daily release, similar to certain earlier graphs, which make it easier to see if MIC is achieved and by what margin, and at what times during the duration of the experiment. For presentation and analysis, the data in the graphs are normalized in certain ways. For the vertical axis on the left side of the graph, the incremental daily release of the drug is considered to be distributed within the pin volume, which results in a concentration of the drug per unit of tissue volume. Corresponding to this is a right-hand vertical axis which is that drug concentration divided by the MIC for a particular bacteria species. This ratio, which is ideally greater than 1 and perhaps greater than 1 by a large factor, indicates the degree of certainty of killing that particular species of bacteria. Each have a right-hand scale that is unique to a particular bacteria species. These graphs show that Blend 9 is expected to be well above the minimum inhibitory concentration for the entirety of the release study.

It can be noted that Blend 9 contains both minocycline and rifampin. If the composition contains only one antibiotic alone, we noticed there were some colonies within the zone of inhibition that became resistant. Accordingly, it is believed that the combination of both drugs prevents development of resistance.

FIGS. 17B-17I show release of drug. FIGS. 17B-17E are for release of Rifampicin. In each of FIGS. 17B-17E, the data are the same and the vertical axis on the left side is the same. However, for each graph the vertical axis on the right side is different because it is associated with the MIC for a particular species of bacteria.

From the graphs it should be noted that the magnitude of the right hand axis is significant for the device's capability of killing each targeted bacteria. For each graph, none fall below the MIC for the entirety of the release and are many times the minimum inhibitory concentration. This set of graphs shows the expected coverage of a fixator pin with a sleeve made blend 9 using its release data.

### Example 12. Measurements of Zone of Inhibition

Experimental results are presented here for zone of inhibition. In a petri dish, a culture of a specified bacterium, which is sometimes referred to as a “lawn” is grown on agar.

Then, a specimen or coupon containing drug-releasing material is placed on the lawn. In the petri dishes presented here, either two or three specimens are placed in a petri dish. The coupon is disc-shaped having a diameter of 0.25 inch (6.35 mm). The release of drug from the coupon is influenced by the release characteristics of the drug+carrier coupon itself, and also by the amount of drug present in the coupon, which in turn may be influenced by how much drug (if any) has already been released from the coupon. In various experiments, some coupons (referred to as time zero) are from as-manufactured material that has never spent any time immersed in a bath. Other coupons are from the same initial material composition after it has been immersed in phosphate buffered saline for a specified period of time such as 18 days. After the coupon is placed on the lawn, the coupon and the lawn are incubated at 37 C and allowed to interact for a period of 24 hours. As a result of that period of interaction, the release of drug from the coupon kills bacteria within an approximately circular region of the lawn, centered around the coupon. This region, which is visibly different from the rest of the lawn in terms of color and other appearance, is called the Zone of Inhibition. It can be measured photographically. The photographs are taken after 24 hours of incubation.

In the photographs, the coupon appears black or some similarly dark color. The coupon, as mentioned, is disc-shaped having a diameter of 0.25 inch (6.35 mm). The petri dish itself, for reference, has a diameter of 100 mm and it has a depth of 15 mm. Incubation is performed for a period of 24 hours at a temperature of 37 C. The Zone of Inhibition is indicated by color change or similar visual indication. The indicated numerical result is the diameter of the Zone of Inhibition. The coupon on the left is an as-manufactured coupon that has been exposed to the drug release bath. The coupon on the right is a coupon that has spent a total of 18 days in the drug release bath prior to being placed in the petri dish. In the petri dishes containing three specimens, the coupon at the top is an as-manufactured coupon that has not been exposed to the drug release bath. The coupon at the lower right is a coupon that has spent a total of 14 days in the drug release bath prior to being placed in the petri dish. The coupon at the lower left is a coupon that has spent a total of 48 days in the drug release bath prior to being placed in the petri dish.

In the photographs presented herein, the “lawn” of culture medium and bacteria is a generally light background color. Each very dark circle is a coupon of the drug-releasing polymeric composition. Surrounding the coupon is an intermediate-colored generally circular region, which is the region in which bacteria are killed, called the Zone of Inhibition. In general, a larger zone of inhibition is more desirable than a smaller zone of inhibition. Culturing to produce the zone of inhibition was done inside an incubator maintaining the temperature at 37 C, and was done for a period of one day after the coupon had been exposed to a bath for the specified number of days.

The photographs are also labeled as to what bacterium is grown on the lawn, and what is the composition of the coupon material.

In some experiments, the bacteria used are E. coli ATCC 25922, and P. aeruginosa ATCC 27853. These are gram negative bacteria and so are especially relevant for applications such as use in catheters and renal applications. The compositions used in these experiments were Blends 9 and 10. The time points are: Day 0; and Day 18.

In other experiments, the bacteria used are: S. aureus ATCC 6538; S. epidermidis ATCC 35984; A. baumannii ATCC 19606.

Petri dishes that contain three specimens show experimental results that are for gram positive bacteria. For a fixator pin, gram positive bacteria are more important. Petri dishes that contain two specimens show experimental results that are for gram negative bacteria.

For petri dishes containing two samples, the photographs show left side is day 0 (coupon as manufactured never having been exposed to the bath); right side is after 18 days releasing. Dimensional measurements of the diameter of the zone of inhibition are taken for two coupons for each condition, and results reported in this table are for two individual specimens and also the average of the two specimens.

This set of experiments shows Blend 9 and Blend 10, interacting with either E. coli or P. aeruginosa, in each possible combination. The data is reported in Table 17.

TABLE 17
Diameter of Zone of Inhibition

| | E. coli ATCC 25922 | | P.aeruginosa ATCC 27853 | |
|---|---|---|---|---|
| | 0 days | 18 days | 0 days | 18 days |
| Blend 9 | 23 mm | 12 mm | 11 mm | 0 mm |
| | 22 mm | 15 mm | 11 mm | 0 mm |
| Blend 9, Average | 22.5 mm | 13.5 mm | 11 mm | 0 mm |
| Blend 10 | 26 mm | 17 mm | 17 mm | 10 mm |
| | 26 mm | 17 mm | 15 mm | 10 mm |
| Blend 10, Average | 26 mm | 17 mm | 16 mm | 10 mm |

Another set of Zone of Inhibition experiments was performed using different bacteria. In this set of experiments, the bacteria used were S. aureus ATCC 6538; S. epidermidis ATCC 35984; and A. baumannii ATCC 19606. These bacteria are especially relevant for applications such as the external fixator. The results are displayed with three samples per petri dish, representing three different time points. In these experiments, the time points for which Zone of Inhibition data are shown are: 0 days in bath; 14 days in bath; and 48 days in bath. In the photos, the plates are organized in a grid by blend number and bacteria. The top coupon is day 0, the bottom right coupon is day 14, and the bottom left coupon is day 48. The compositions used in these experiments were Blends 6, 7, 8, 9, 10, 11, 12, 13, 14 and 15.

This set of experiments shows, with the three different bacteria, the effect of polymer additives. Blend 13 contains only Elvax 40W, with neither PEG nor PCL. Blend 11 contains Elvax 40W with PEG but no PCL. Blend 12 contains Elvax 40W with PCL but no PEG. Bend 8 contains Elvax 40W with both PEG and PCL. The data is reported in Table 18.

TABLE 18

| | | | | | | Linearized | Total | Linearized | Total |
|---|---|---|---|---|---|---|---|---|---|
| | | | | | | Release | Release at | Release | Release at |
| | | | | | | rate MINO | day 30 | rate RIF | day 30 |
| Blend | EVA | PEG | PCL | RIF | MINO | [ug/day1/2] | MINO | [ug/day1/2] | RIF[ug] |
| 8 | 70% | 10% | 10% | 5% | 5% | 131.49 | 663 | 73.40 | 378 |
| 11 | 80% | 10% | 0% | 5% | 5% | 52.02 | 350 | 51.11 | 272 |
| 12 | 80% | 0% | 10% | 5% | 5% | 31.96 | 141 | 40.54 | 187 |
| 13 | 90% | 0% | 0% | 5% | 5% | 24.09 | 160 | 27.15 | 148 |

### Effect of Increasing API Concentration

This set of experiments, for Elvax 40W with the same concentrations of PEG (10%) and PCL (10%), shows the effect of increasing concentration of both minocycline and rifampin (with equal concentrations of each drug). The data is reported in Table 19.

TABLE 19

| | | | | | | Linearized | Total | Linearized | Total |
|---|---|---|---|---|---|---|---|---|---|
| | | | | | | Release | Release at | Release | Release at |
| | | | | | | rate MINO | day 30 | rate RIF | day 30 |
| Blend | EVA | PEG | PCL | RIF | MINO | [ug/day1/2] | MINO | [ug/day1/2] | RIF[ug] |
| 8 | 70% | 10% | 10% | 5% | 5% | 131.49 | 663 | 73.40 | 378 |
| 9 | 60% | 10% | 10% | 10% | 10% | 409.05 | 2097 | 195.93 | 1055 |
| 10 | 40% | 10% | 10% | 20% | 20% | 399.13 | 5992 | 559.74 | 5415 |

In regard to concentration of drug in the compositions, an intuitive expectation might be that larger drug loading should give larger release. This appears true from a comparison of the data of 5% concentration of each drug (Blend 8) and 10% concentration of each drug (Blend 9). However, from these results (looking at Blend 10, which contains a 20% concentration of each drug), it also seems that too large a drug concentration can be undesirable, in that a drug concentration can be undesirably large such that by the 48-day point, the Zone of Inhibition is vanishingly small, even though the samples started out containing the largest concentration of the drugs. In this situation it perhaps appears that the drug has already depleted from the coupons. This is consistent with the observations in the release studies using these same compositions.

### Effect of Grinding of Rifampin

The data is reported in Table 20.

TABLE 20

| | | | | | | Linearized | Total | Linearized | Total |
|---|---|---|---|---|---|---|---|---|---|
| | | | | | | Release | Release at | Release | Release at |
| | | | | | | rate MINO | day 30 | rate RIF | day 30 |
| Blend | EVA | PEG | PCL | RIF | MINO | [ug/day1/2] | MINO | [ug/day1/2] | RIF |
| 8 | 70% | 10% | 10% | 5% | 5% | 131.49 | 663 | 73.40 | 378 |
| 10 | 40% | 10% | 10% | 20% | 20% | 399.13 | 5992 | 559.74 | 5415 |
| 14 | 40% | 10% | 10% | 20%** | 20% | 400.86 | 6972 | 475.00 | 5073 |
| 15 | 70% | 10% | 10% | 5%** | 5% | 138.13 | 694 | 55.07 | 315 |

**ground

In these experiments, the compositions containing unground rifampicin were Blend 8 (at 5% concentration of unground rifampicin) and Blend 10 (at 20% concentration of unground rifampicin). The compositions containing ground rifampicin were Bend 15 (at 5% concentration of ground rifampicin) and Blend 14 (at 20% concentration of ground rifampicin). In all compositions, minocycline was present at a concentration equal to the rifampicin concentration. These experiments seem to show that at 5% rifampin concentration, the unground and the ground rifampin produce very similar results. At 20% rifampin concentration, the results are similar to the results for the lower concentrations of rifampin, except that at the longest-duration data point there is some difference in the appearance of the zone of inhibition.

### Effect of Combination of API

The data is reported in Table 21.

TABLE 21

| | | | | | | Linearized | Total | Linearized | Total |
|---|---|---|---|---|---|---|---|---|---|
| | | | | | | Release | Release at | Release | Release at |
| | | | | | | rate MINO | day 30 | rate RIF | day 30 |
| Blend | EVA | PEG | PCL | RIF | MINO | [ug/day1/2] | MINO | [ug/day1/2] | RIF[ug] |
| 6 | 75% | 10% | 10% | 5% | 0% | — | — | 28.53 | 143 |
| 7 | 75% | 10% | 10% | 0% | 5% | 1.62 | 59 | — | — |
| 8 | 70% | 10% | 10% | 5% | 5% | 131.49 | 663 | 73.40 | 378 |

In this set of experiments, the drugs present in the composition were either rifampin only (at a 5% concentration) (Blend 6); minocycline only (at a 5% concentration) (Blend 7); or both rifampin and minocycline (each at a 5% concentration) (Blend 8).

Table 22 is a summary of the measured diameters of the zones of inhibition for various coupon compositions, time points and bacteria.

TABLE 22
Measured Diameter of Zone of Inhibition

| | S. aureus | | | S. epidermidis | | | A. baumannii | | |
|---|---|---|---|---|---|---|---|---|---|
| | ATCC 6538 | | | ATCC 35984 | | | ATCC 19606 | | |
| | 0 | 14 | 48 | 0 | 14 | 48 | 0 | 14 | 48 |
| | days | days | days | days | days | days | days | days | days |
| Blend | 29 mm | 24 mm | 21 | 35 | 31 mm | 26 | 14 | 8 mm | 0 mm |
| 6 | | | mm | mm | | mm | mm | | |
| Blend | 26 mm | 16 mm | 16 | 27 | 18 mm | 17 | 26 | 17 mm | 16 |
| 7 | | | mm | mm | | mm | mm | | mm |
| Blend | 26 mm | 23 mm | 20 | 35 | 39 mm | 25 | 27 | 17 mm | 12 |
| 8 | | | mm | mm | | mm | mm | | mm |
| Blend | 28 mm | 23 mm | 26 | 34 | 27 mm | 30 | 29 | 15 mm | 14 |
| 9 | | | mm | mm | | mm | mm | | mm |
| Blend | 29 mm | 28 mm | 9 | 37 | 34 mm | 10 | 30 | 16 mm | 0 mm |
| 10 | | | mm | mm | | mm | mm | | |
| Blend | 28 mm | 24 mm | 9 | 36 | 27 mm | 11 | 26 | 15 mm | 0 mm |
| 11 | | | mm | mm | | mm | mm | | |
| Blend | 27 mm | 23 mm | 21 | 32 | 26 mm | 23 | 25 | 17 mm | 12 |
| 12 | | | mm | mm | | mm | mm | | mm |
| Blend | 25 mm | 24 mm | 22 | 32 | 26 mm | 24 | 25 | 21 mm | 17 |
| 13 | | | mm | mm | | mm | mm | | mm |
| Blend | 29 mm | 32 mm | 23 | 40 | 37 mm | 27 | 32 | 18 mm | 19 |
| 14 | | | mm | mm | | mm | mm | | mm |
| Blend | 29 mm | 25 mm | 23 | 36 | 28 mm | 27 | 28 | 17 mm | 14 |
| 15 | | | mm | mm | | mm | mm | | mm |

### Example 13: Shapes of Particles of Each Drug

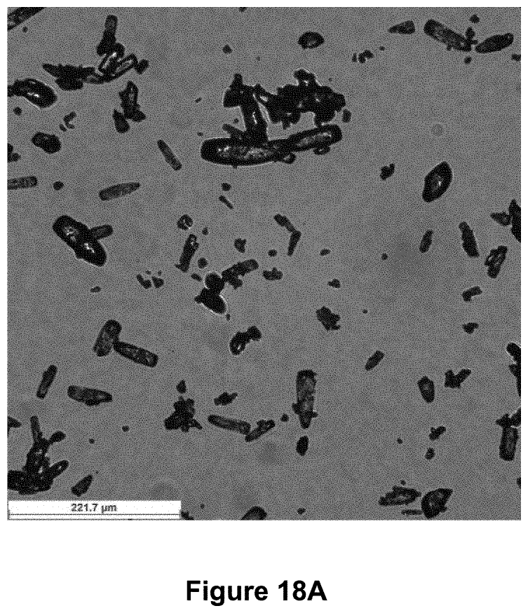

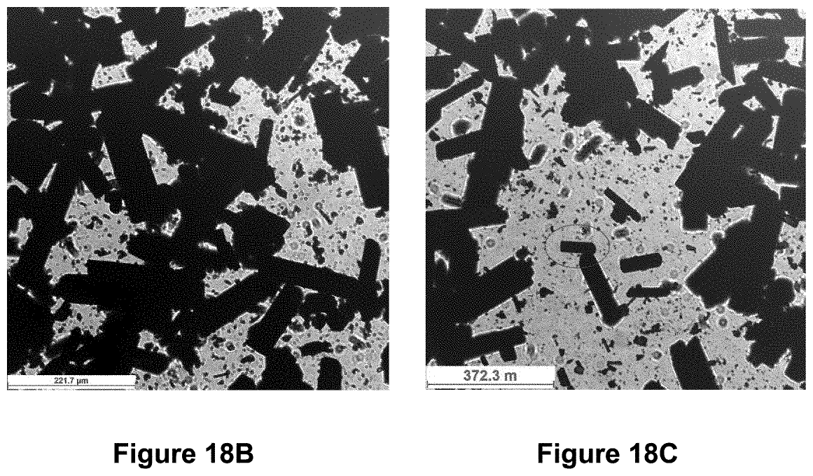

FIG. 18A shows particles of minocycline (prior to being used to make a composition of an embodiment of the invention). The minocycline particles are generally spherical or ellipsoidal. The smaller particles generally are spherical and the larger particles are ellipsoidal. FIGS. 18B and 18C: show rifampin crystals spread on a glass slide, prior to the time of manufacturing the composition of an embodiment of the invention. The rifampin particles have the form of elongated rectangles with sharp edges, as shown at two different magnifications.

The minocycline particles are generally smaller particles than the rifampicin particles. It appears that the minocycline particles are, at most, about half the size of the rifampicin particles.

### Example 14: Rounding of Initially-Sharp Corners of Rifampin Implying that there is Formation of Solid Solution

As a qualitative observation, it is possible to make photographic comparisons that lead to the conclusion that there is some dissolution of drug particles into the polymeric carrier. In this observation, the drug that is photographed is rifampicin. The particles of rifampicin are irregular and plate-like or crystal-like in shape, and, specifically, have sharp corners.

It is believed that, if mass is leaving the drug particles and is going into solution in the polymeric carrier, that is favorable for mass transport. This is due to an increase in the surface area available for transport as well as the drug being more readily available to desorp from the polymer than from a crystalline form. If this is happening, it would result in initially sharp-cornered particles becoming more rounded at their corners. It is believed that this is observed, as shown in the following photographs.

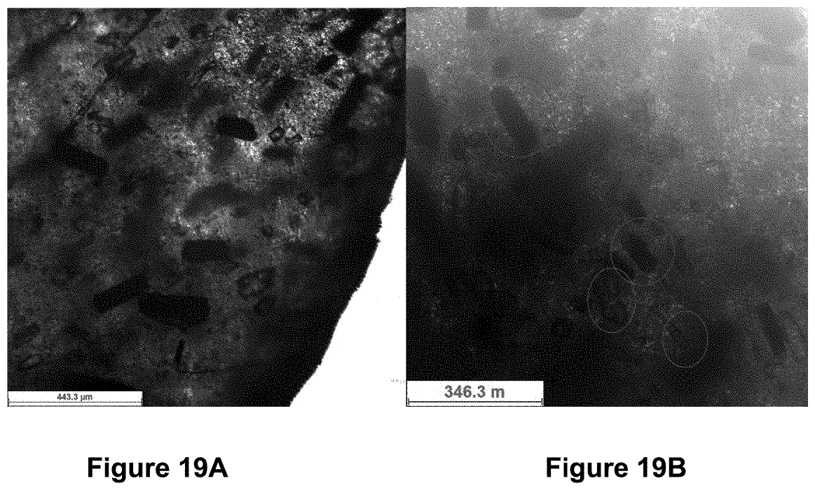

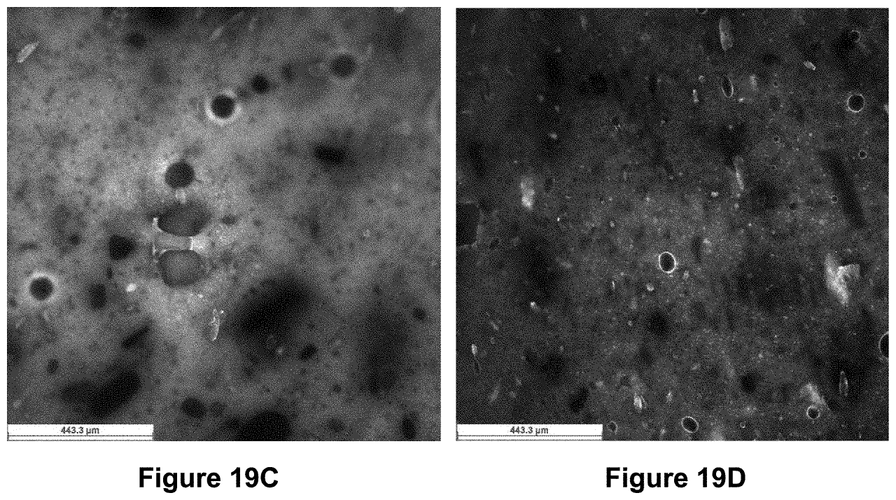

FIGS. 18B and 18C show rifampin crystals spread on a glass slide, prior to the time of manufacturing the composition of an embodiment of the invention. The rifampin particles have the form of elongated rectangles with sharp edges, as shown at two different magnifications. FIGS. 18B and 18C show that the rifampin drug was provided (prior to being mixed into the composition) in the form of relatively large plate-like particles that had sharp corners. Later, after the composition was processed for ˜10 minutes at 140 C (which is above the melting point of the plastics but well below the melting point of the drugs), similar plates were visible in micrographs but the corners of the plates were more rounded. We think that the roundedness indicates that dissolution has occurred, occurring preferentially at the corners, edges, and smaller crystals (which may be totally dissolved) for reasons that have to do with surface energy. Perhaps the drug is going into solid solution. This is shown in FIGS. 19A, 19B. FIG. 19B also shows that the rifampicin is breaking apart in the bottom right red circle. It is believed that when some of the rifampin becomes a part of the polymer host, rather than staying in its own crystals, it becomes easier to release from the polymer host. This is because the surface area where mass transport occurs increases and drug is more readily available from the polymer compared to the crystals. The pores are significant as physically demonstrating release of drug. Pore formation increases surface area and speeds release of drug as well.

FIG. 19A, 19B show Polymer blend Before releasing. Rounded edges of rifampin are highlighted in FIG. 19B. The rounded edges of the rifampicin crystals demonstrate interaction with host polymer, and solubility. FIG. 19C, 19D show Polymer blends after releasing for 30 days. This demonstrates pore formation during release. This also demonstrates that rifampicin crystals remain even after this period of time.

We know that at the processing temperature (140° C.), both minocycline and rifampin are stable and the processing temperature is well below the melting point of both drugs, so the corner-rounding is not likely to be due to degradation. The corner-rounding is believed to happen during the mixing while the rifampin is exposed to 140° C.

### Example 15: Comparison of Minocycline Hydrochloride and Minocycline Free Base

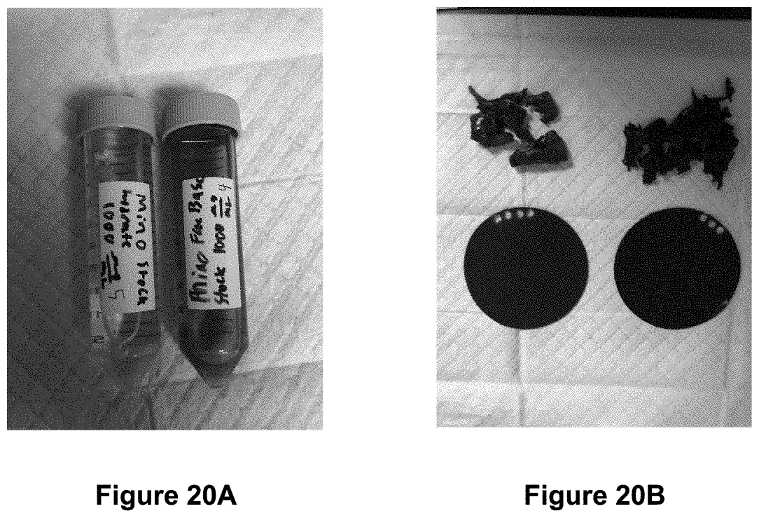

Photographic results about the stability of the two forms of minocycline are shown in FIGS. 20A, 20B. The experiment is about the possibility that there could be some degradation of the minocycline or formation of some other compound. When the minocycline is in the form of minocycline free base, it seems like the drug oxidizes with the passage of time, based on discoloration (darkening) of the samples. This occurs both when the minocycline free base is simply stored dissolved in distilled water and also when the minocycline free base is formulated in compositions of the invention. This discoloration process happens more quickly when the minocycline free base is stored in water than when the minocycline free base is in solid form in the host polymer. This discoloration process is not observed at all when the minocycline free base is in dry powder form exposed to air. When the drug is in the form of the minocycline hydrate, stability seems to be better than for minocycline free base, as indicated by less discoloration or darkening. This observation applies both when the minocycline hydrate is simply stored in distilled water and when the minocycline hydrate is formulated in compositions of the invention. In most of the experiments reported herein, the minocycline was in the form of minocycline hydrochloride. Minocycline hydrochloride is more readily available commercially, and, as we found through testing, it is less prone to discoloration. Also, for long-term release, we found that the Free Base is less desirable for long term release, although it still may be suitable for short and medium term release applications. If there were a special need for a strong bacteria-killing or anti-inflammatory release, especially early or quickly, it might be appropriate to use the free base version of minocycline.

### Example 16: Thermogravimetric Analysis

Thermogravimetric analysis was performed on the APIs used in compositions. Each drug was ramp heated to 140° C. and held at that temperature for 10 minutes. For all compositions, time spent melt-mixing is 8-10 minutes. The decomposition of the API in that time was less than 0.5% for each drug, meaning that the API are stable during processing. For Rifampin, the change in mass was 0.292%; for Minocycline HCl the change in mass was 0.421%; and for minocycline Free Base the change in mass was 0.137%.

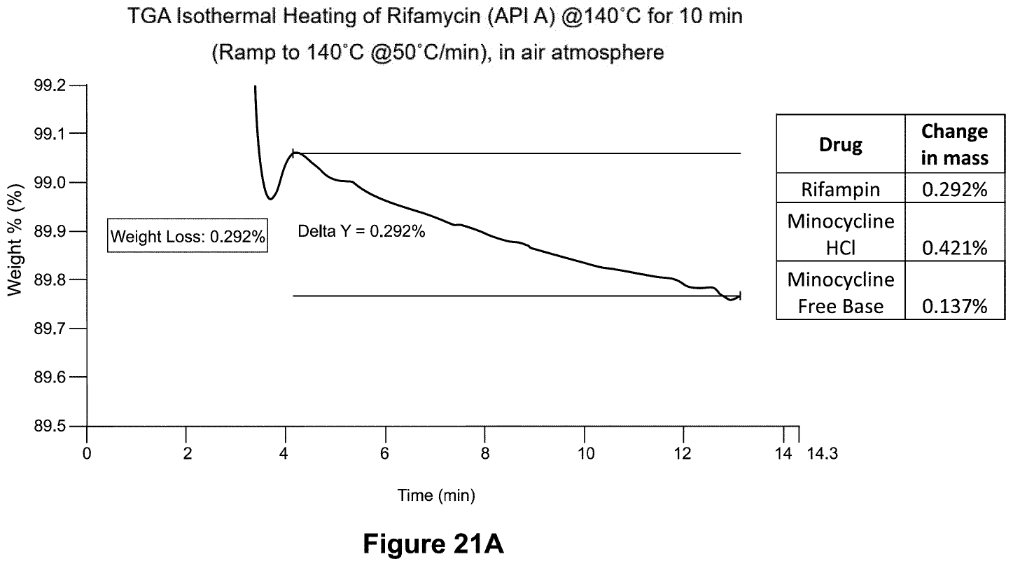

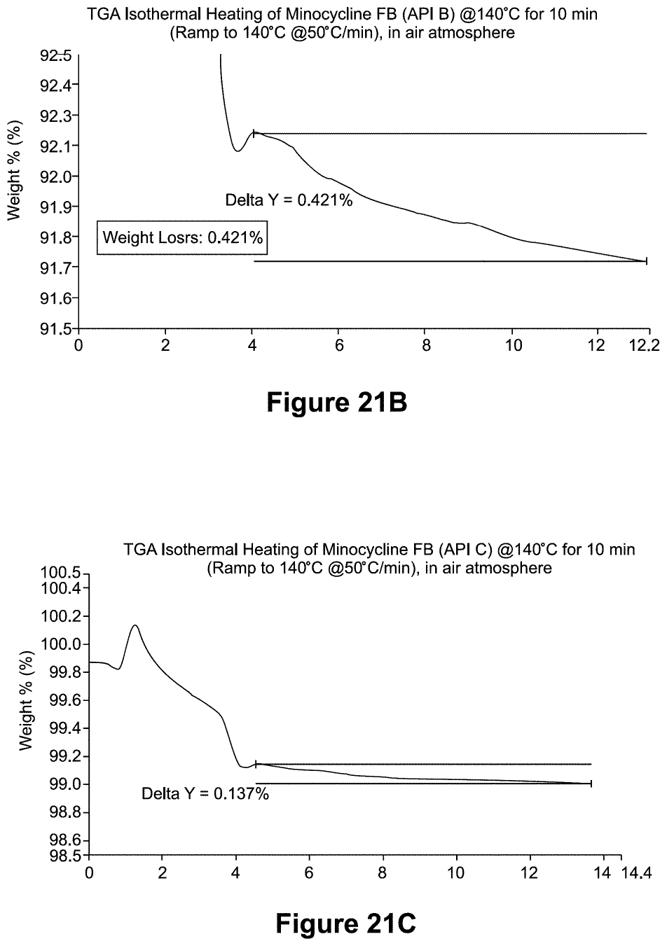

Curves for each drug are presented in FIGS. 21A, 21B, 21C showing the weight loss of API over the 10 minutes at processing temperature.

### Further Comments

In embodiments of the invention, in addition to the already-described host material comprising ethylene vinyl acetate copolymer, other host materials are also possible. Another example of such a possible host polymeric material is polyurethane (PU) and composites that include polyurethane. The polyurethane family includes (but is not limited to) Aliphatic and Aromatic chemistries, as well as carbonate based chemistries and Polyether based chemistries. These various chemistries can provide various different biocompatible options with beneficial physical properties relating to degradation and rigidity.

Still other examples of possible polymeric host material may include silicones, poly(acrylates), and other copolymers. This group of polymer host materials may be combined with the other components described herein, such as release modifying polymer additives and various drug combinations. Any embodiment could include host polymers and additives that are either resorbable or non-resorbable.

A still further list of possible polymers for use in the composition or in the device according to the present disclosure, and which may be the major component, includes:
EVA (various ratios)
Polyurethanes (aliphatic and aromatic chemistries)
Polyurethanes with Carbonate and polyether based chemistries
Composites containing polyurethanes
Silicones
Poly(acrylates)
Polyethylene
Ultrahigh molecular weight polyethylene
Polylactic acid
Polyglycolic acid
Poly lactic co-glycolic acid
Acrylic
Nylon
Acetal
Polyester
Polytetrafluoroethylene
Polyvinyl chloride
Polydimethyl siloxane
PMMA (Polymethyl methacrylate)
Polypropylene
PET (Polyethylene terephthalate or Polyethylene terephthalate glycol-modified)
Polyamides
Polyesters
PEEK (polyetheretherketone)
Polycarbonates
Polysulfone
Polyetherimide
Polycaprolactone
Polyethylene glycol
Polyethylene oxide
Polyvinyl alcohol
poly(N-vinylpyrrolidone)
Polyvinyl acetate
Poloxamer

In embodiments of the invention, in addition to the already-described drugs, other possible drugs that could be used include:
Ciprofloxacin (and possibly other fluoroquinolones)
Vancomycin (and other glycopeptides)
Tetracyclines class antibiotics
Cephalosporins (for more gram negative applications)
Penicillins
Gentamicin (and other aminoglycosides)
Sulfonamides
Macrolides
Carbapenems
Other antimycobacterial drugs (in addition to rifampicin)
Immunosuppressants

In embodiments of the invention, here are some possible types of implants and applications of the inventive Controlled Release Polymer:
External fixator pin cover
Molded sleeve around K-wire
Other fixators that are transdermal
Other devices designed for fixation such as mandibular fixators and elbow fixators
Transcutaneous catheter
Orthopedic surgical equipment
Sleeves for orthopedic surgical equipment
Pods or other shapes to be incorporated into prosthesis
Orthopedic implants
Sleeve/pouch for breast implants and other implants
Urinary catheter
Intra-Uterine devices (IUDs) and Ancillary equipment for IUDs
Catheter lock/plug
Ancillary Catheter equipment such as molded fittings and connectors
Implants
Wound cover
Dermal applications such as patches where controlled release of anti-inflammatory compounds and antibiotics would be beneficial
Covers of implantable screws, rods, discs plates for traumatic fracture repair and other
Ancillary equipment for cardioverter defibrillators
Drug impregnated Coronary stents
Covers and guards for medical equipment
Incorporation in tracheal tubes for anesthesia and breathing pathways
Vascular graft prosthesis
Antibacterial tubing and guards for cardiopulmonary bypass, dialysis, and ECMO (extracorporeal membrane oxygenation) machines (long procedures that could use antibacterial protection; ECMO in particular has long term use)
Drug releasing polymer mesh bag for pacemaker
Tack cover release for sacculotomy
Endolymphatic shunts and ear tubes, tympanostomy tubes
Ear mold/ear plugs for swimmers to prevent swimmers ear infections
Endovascular shunt adaptors
Suture covers/the sutures themselves (polypropylene and polyethylene are used in sutures so it stands to reason there could be controlled release sutures)
Implantable clips
Burr hole cover for neurosurgery
Fallopian tube prosthesis/insert
Long term intravascular catheter
Incorporation in breast implants
Incorporation into prosthetic
A still further list of implantable items, compiled by the US Food and Drug Administration, is given at https://www.fda.gov/media/85192/download

Compositions of embodiments of the invention could be used with or to make any kind of catheter, such as: central venous catheter; triple lumen catheter; a catheter for neurosurgery; external ventricular drains; ventricular peritoneal shunt.

Compositions of embodiments of the invention could be used with transcutaneous driveline devices for myocardial assist devices, either as a sleeve where the driveline penetrates the skin or as a material of which the driveline itself is made of or comprises.

Also, compositions of embodiments of the invention could be used with devices that are fully implanted rather than transcutaneous. For example, breast implants may comprise an enclosure made of silicone, which encloses a liquid. The enclosure may be made of a drug-loaded silicone composition of embodiments of the invention. The embodiment may be used not just to deliver drug but to prevent capsular contraction, i.e., the formation of scar tissue, because bacteria on the surface of the implant may cause chronic capsular contraction. Also, it is possible that anaplastic large cell lymphoma may be caused by bacteria on the surface of the implant, so the inventive composition may serve a role as an antineoplastic, in addition to combatting infection.

Other examples of fully implanted devices that could use compositions of embodiments of the invention, as a coating or as any portion of the device, include: cardiac pacemakers; intraventricular pumps; implantable pumps for delivering anesthetic, insulin, chemotherapy drugs or other drugs; and glucose monitors that are implanted under the skin.

Orthopedic implants may contain pods of the composition of embodiments of the invention, such as small pieces that may be press-fitted into a cavity in the implant, even if much of the overall surface of the implant is left bare for purposes of interacting with bone. The pods may be placed at implant locations that are not highly stressed, so that their placement would not significantly weaken the component in which they are placed. Some orthopedic joint replacement implants comprise a metal part, and another metal part, and a polymeric part between the two metal parts, with the polymeric part serving as a surface that is involved in articulation. The pods may be located near the polymeric part. In yet another embodiment, the polymeric part may itself be made of a composition of an embodiment of the invention, such as a drug-loaded polyurethane or a drug-loaded ultra high molecular weight polyethylene. A part of some knee implants may be an artificial patella, which is believed to experience mechanical loads that are not severe. The articulating (posterior-facing) surface of an artificial patella could be made of a composition of an embodiment of the invention.

Embodiments of the invention could be used to make an endotracheal tube. (The antibiotics used could be other than minocycline and rifampin.)

Embodiments of the invention could be used to make antimicrobial sutures.

In general, although minocycline and rifampin have been used in the experiments described herein, other antibiotics could also be used.

In embodiments of the invention, the composition of the invention can be dispersed and incorporated in another polymer to function as drug releasing entities. The composition of the invention can be made in the form of small particles to be implanted or placed around an implanted prosthesis/device. The composition of the invention can be made in the form of a sleeve to wrap around an implanted prosthesis or device or can be formed to wrap around or partially surround an organ or nerve or blood vessel or other anatomic element.

In general, any combination of disclosed features, components and methods described herein, that is physically possible, is intended to be within the scope of the claims.

All cited references are incorporated by reference herein.

Although embodiments have been disclosed, it is not desired to be limited thereby. Rather, the scope should be determined only by the appended claims.

We claim:

1. A composition comprising:
a host polymeric material making up a major portion of said composition;
a first release-modifying material, said first release-modifying material being mixed together with said host polymeric material, said first release-modifying material making up a minor portion of said composition;
a second release-modifying material, said second release-modifying material being different from said first release-modifying material, said second release-modifying material being mixed together with said host polymeric material, said second release-modifying material making up a minor portion of said composition;
a first drug; and
a second drug,
wherein at least some of said first drug is present in the form of discrete first particles within said composition, and at least some of said second drug is present in the form of discrete second particles within said composition,
wherein each of said drugs has a respective cumulative release of said drug from said composition to an aqueous environment that is greater than would occur with an identical composition absent said second release-modifying material.

2. The composition of claim 1, wherein said host polymeric material and said first release-modifying material are less than fully miscible with each other.

3. The composition of claim 1, wherein said host polymeric material and said second release-modifying material are less than fully miscible with each other.

4. The composition of claim 1, wherein said first release-modifying material or said second release-modifying material is a material that is more hydrophilic than said host polymeric material.

5. The composition of claim 1, wherein said host polymeric material comprises ethylene vinyl acetate copolymer.

6. The composition of claim 1, wherein said first release-modifying material comprises polyethylene glycol.

7. The composition of claim 1, wherein said second release-modifying material comprises polycaprolactone.

8. The composition of claim 1, wherein a concentration of said first release-modifying material equals, or is within 50% to 200% of, a concentration of said second release-modifying material.

9. The composition of claim 1, wherein said first drug is more water-soluble and said second drug is less water-soluble.

10. The composition of claim 1, wherein said first drug is more soluble in water than said second drug by a factor of at least 10, and respective concentrations of said first drug and said second drug in said composition are within 50% to 200% of each other.

11. The composition of claim 1, wherein said first drug or said second drug or both of said drugs are suitable to form a solid solution to at least some extent in said host polymeric material.

12. The composition of claim 1, wherein one of said more-soluble drug and said less-soluble drug is effective against gram-positive bacteria and the other of said drugs is effective against gram-negative bacteria.

13. The composition of claim 1, wherein one of said drugs has an anti-inflammatory property.

14. The composition of claim 1, wherein one of said drugs has both an anti-bacterial property and an anti-inflammatory property.

15. The composition of claim 1, wherein said first drug is minocycline and said second drug is rifampin.

16. The composition of claim 1, wherein said first particles of said first drug are in a form of nearly-spherical particles.

17. The composition of claim 1, wherein said wherein said second particles of said second drug are in a form of crystals.

18. The composition of claim 1, wherein said first particles of said first drug are smaller than said second particles of said second drug.

19. The composition of claim 1, wherein a concentration of said first drug in said composition and a concentration of said second drug in said composition are equal to each other or are within a range of 50% to 200% of each other.

20. The composition of claim 1, wherein said composition is capable of providing drug release to said aqueous environment at a clinically effective concentration for a time duration of at least 40 days.

21. The composition of claim 1, wherein said composition is melt-processable.

22. The composition of claim 1, wherein for said composition there exists a processing temperature at which said first drug and said second drug do not fully melt and said first drug and said second drug experience less than 5% degradation upon exposure to said processing temperature for a period of 10 minutes, said processing temperature being a temperature at which said host polymeric material is soft enough to be extruded.

23. The composition of claim 1, wherein said composition is formed into a sleeve suitable to be disposed in a vicinity of a skin-penetrating medical device or an implantable medical device.

24. The composition of claim 23, wherein said skin-penetrating medical device is a fixator pin or a catheter.

25. A sleeve comprising the composition of claim 1, wherein said sleeve is suitable to be disposed around an object that penetrates through the skin of a patient.

26. A sleeve comprising the composition of claim 1, wherein said sleeve is suitable to be disposed around an external fixator pin that penetrates through the skin of a patient.

27. The composition of claim 1, wherein said host polymeric material comprises silicone.

28. The composition of claim 1, wherein said host polymeric material comprises polyurethane.

29. A composition comprising:
a host polymeric material making up a major portion of said composition;
at least one release-modifying material, said first release-modifying material being mixed, together with said host polymeric material, said first release-modifying material making up a minor portion of said composition;
a first drug; and
a second drug,
wherein at least some of said first drug is present in the form of discrete first particles within said composition, and at least some of said second drug is present in the form of discrete second particles within said composition,
wherein said first drug has a respective cumulative release of said first drug from said composition to an aqueous environment that is greater than would occur with an identical composition absent said second drug, and
wherein said second drug has a respective cumulative release of said second drug from said composition to an aqueous environment that is greater than would occur with an identical composition absent said first drug.

30. The composition of claim 29, further comprising a second release-modifying material, said second release-modifying material being different from said first release-modifying material, said second release-modifying material being mixed together with said host polymeric material, said second release-modifying material making up a minor portion of said composition.

31. The composition of claim 29, wherein said host polymeric material comprises ethylene vinyl acetate copolymer.

32. The composition of claim 29, wherein said first release-modifying material comprises polyethylene glycol.

33. The composition of claim 29, wherein said second release-modifying material comprises polycaprolactone.

34. A composition comprising:
a host polymeric material making up a major portion of said composition, said host polymeric material being a silicone; and
a first drug,
wherein at least some of said first drug is present in the form of discrete first particles within said composition.

35. The composition of claim 34, further comprising a second drug that is present in the form of discrete second particles within said composition.

36. The composition of claim 35, wherein said first drug is rifampin and said second drug is minocycline.

37. A device for implanting in body tissue and having antibacterial properties comprising:
an implantable device having an exterior surface and being configured for implanting in body tissue, wherein at least a portion of the exterior surface comprises a composition comprising:
a host polymeric material making up a major portion of said composition;
a first release-modifying material, said first release-modifying material being mixed together with said host polymeric material, said first release-modifying material making up a minor portion of said composition;
a second release-modifying material, said second release-modifying material being different from said first release-modifying material, said second release-modifying material being mixed together with said host polymeric material, said second release-modifying material making up a minor portion of said composition;
a first drug; and
a second drug,
wherein at least some of said first drug is present in the form of discrete first particles within said composition, and at least some of said second drug is present in the form of discrete second particles within said composition,
wherein each of said drugs has a respective cumulative release of said drug from said composition to an aqueous environment that is greater than would occur with an identical composition absent said second release-modifying material.

38. The device according to claim 37, wherein:
(a) the device comprises a fixation pin.

39. The device according to claim 37, wherein:
(a) the device comprises a catheter lock plug.

40. The device according to claim 37, wherein:
(a) the device comprises a tympanostomy tube.
